SEQUENCE LISTING (44 sequences)

SEQ ID NO: 1 - DNA, 20 bp, synthetic construct
  source 1..20: mol_type = other DNA
tctggagctggtggagccct

SEQ ID NO: 2 - DNA, 24 bp, synthetic construct
  source 1..24: mol_type = other DNA
agtctagtttctgcctgaccttgg

SEQ ID NO: 3 - DNA, 21 bp, synthetic construct
  source 1..21: mol_type = other DNA
tcctgccagttcgagtcctat

SEQ ID NO: 4 - DNA, 21 bp, synthetic construct
  source 1..21: mol_type = other DNA
aatgacgacagcctttccatc

SEQ ID NO: 5 - DNA, 23 bp, synthetic construct
  source 1..23: mol_type = other DNA
cttccaggggataaaggacttgt

SEQ ID NO: 6 - DNA, 22 bp, synthetic construct
  source 1..22: mol_type = other DNA
acataggcaccaccacattcta

SEQ ID NO: 7 - DNA, 21 bp, synthetic construct
  source 1..21: mol_type = other DNA
aggctggagagctacaagagg

SEQ ID NO: 8 - DNA, 21 bp, synthetic construct
  source 1..21: mol_type = other DNA
cccattccttcttggggtcag

SEQ ID NO: 9 - DNA, 20 bp, synthetic construct
  source 1..20: mol_type = other DNA
tgcccttgctgttcttctct

SEQ ID NO: 10 - DNA, 20 bp, synthetic construct
  source 1..20: mol_type = other DNA
gtggaatcttccggctgtag

SEQ ID NO: 11 - DNA, 20 bp, synthetic construct
  source 1..20: mol_type = other DNA
aagctgccgggaggtgtaag

SEQ ID NO: 12 - DNA, 23 bp, synthetic construct
  source 1..23: mol_type = other DNA
tgtctgccctctctctcctcttg

SEQ ID NO: 13 - DNA, 22 bp, synthetic construct
  source 1..22: mol_type = other DNA
tgcccacgtcaaggagtatttc

SEQ ID NO: 14 - DNA, 27 bp, synthetic construct
  source 1..27: mol_type = other DNA
tcctagctcatctccaaatagttgatg

SEQ ID NO: 15 - DNA, 20 bp, synthetic construct
  source 1..20: mol_type = other DNA
ccgtatccgagtagcttcgc

SEQ ID NO: 16 - DNA, 22 bp, synthetic construct
  source 1..22: mol_type = other DNA
tcaatggagaatttgcccatgt

SEQ ID NO: 17 - DNA, 20 bp, synthetic construct
  source 1..20: mol_type = other DNA
gggcgaagaaaaccgcatca

SEQ ID NO: 18 - DNA, 19 bp, synthetic construct
  source 1..19: mol_type = other DNA
tcaccatcccgcatctcca

SEQ ID NO: 19 - DNA, 20 bp, synthetic construct
  source 1..20: mol_type = other DNA
ggtttcaacgccgactacga

SEQ ID NO: 20 - DNA, 21 bp, synthetic construct
  source 1..21: mol_type = other DNA
gttggcactagagacggacag

SEQ ID NO: 21 - DNA, 23 bp, synthetic construct
  source 1..23: mol_type = other DNA
gccaccttttgacagtgatgaga

SEQ ID NO: 22 - DNA, 20 bp, synthetic construct
  source 1..20: mol_type = other DNA
atcaggacagcccaggtcaa

SEQ ID NO: 23 - DNA, 20 bp, synthetic construct
  source 1..20: mol_type = other DNA
gccttcttgggactgatgct

SEQ ID NO: 24 - DNA, 23 bp, synthetic construct
  source 1..23: mol_type = other DNA
gccattgcacaactcttttctca

SEQ ID NO: 25 - DNA, 23 bp, synthetic construct
  source 1..23: mol_type = other DNA
gagaagcatggcccagaaatcaa

SEQ ID NO: 26 - DNA, 23 bp, synthetic construct
  source 1..23: mol_type = other DNA
aaaatcactcttcacctgctcca

SEQ ID NO: 27 - DNA, 20 bp, synthetic construct
  source 1..20: mol_type = other DNA
ccttctacgacgatgccctc

SEQ ID NO: 28 - DNA, 22 bp, synthetic construct
  source 1..22: mol_type = other DNA
ggttcaaggtcatgctctgttt

SEQ ID NO: 29 - DNA, 21 bp, synthetic construct
  source 1..21: mol_type = other DNA
aatcggctcacaagggattca

SEQ ID NO: 30 - DNA, 23 bp, synthetic construct
  source 1..23: mol_type = other DNA
tcctcccagcttaaagattttgg

SEQ ID NO: 31 - DNA, 22 bp, synthetic construct
  source 1..22: mol_type = other DNA
cggacagggaaatctatggtgc

SEQ ID NO: 32 - DNA, 21 bp, synthetic construct
  source 1..21: mol_type = other DNA
tggtagcgccaggtaaaagag

SEQ ID NO: 33 - DNA, 20 bp, synthetic construct
  source 1..20: mol_type = other DNA
aaccccgaaaggtgtggatg

SEQ ID NO: 34 - DNA, 20 bp, synthetic construct
  source 1..20: mol_type = other DNA
tggccccagggatgatctaa

SEQ ID NO: 35 - DNA, 21 bp, synthetic construct
  source 1..21: mol_type = other DNA
ccgcaaaacagactagccaac

SEQ ID NO: 36 - DNA, 20 bp, synthetic construct
  source 1..20: mol_type = other DNA
caagcccatgtctttggcac

SEQ ID NO: 37 - DNA, 21 bp, synthetic construct
  source 1..21: mol_type = other DNA
ggaccatgactgcaaggactg

SEQ ID NO: 38 - DNA, 21 bp, synthetic construct
  source 1..21: mol_type = other DNA
ccgttgaaggtgagtacagcc

SEQ ID NO: 39 - DNA, 20 bp, synthetic construct
  source 1..20: mol_type = other DNA
tcgcagggagttcgcaaaag

SEQ ID NO: 40 - DNA, 20 bp, synthetic construct
  source 1..20: mol_type = other DNA
acccagcaagaaccctttcc

SEQ ID NO: 41 - DNA, 25 bp, synthetic construct
  source 1..25: mol_type = other DNA
ctgctattcaggctcactacaagag

SEQ ID NO: 42 - DNA, 21 bp, synthetic construct
  source 1..21: mol_type = other DNA
ctctgtctttggggtggttgg

SEQ ID NO: 43 - DNA, 21 bp, synthetic construct
  source 1..21: mol_type = other DNA
gctgatcttcccacaggagac

SEQ ID NO: 44 - DNA, 22 bp, synthetic construct
  source 1..22: mol_type = other DNA
gagctaacctctggcacttctc

(19) United States
(12) Patent Application Publication
(10) Pub. No.: US 2026/0166017 A1
(43) Pub. Date: Jun. 18, 2026

(54) SARM1 INHIBITORS FOR USE IN THERAPY AND COSMETICS

(71) Applicant: CAMBRIDGE ENTERPRISE LIMITED, Cambridge Cambridgeshire (GB)

(72) Inventors: Peter James ARTHUR-FARRAJ, Cambridge Cambridgeshire (GB); Andrea LORETO, Cambridge Cambridgeshire (GB)

(21) Appl. No.: 19/129,078

(22) PCT Filed: Nov. 10, 2023
(86) PCT No.: PCT/GB2023/052956

§ 371 Date: May 12, 2025

(30) Foreign Application Priority Data
Nov. 12, 2022 (GB) 2216910.6
Nov. 28, 2022 (GB) 2217810.7

(51) Int. Cl. A61K 31/4406; A61K 8/49; A61K 8/60; A61K 8/67; A61K 31/4402; A61K 31/455; A61K 31/706; A61K 45/06; A61P 25/00; A61Q 19/00; A61Q 19/08

(52) U.S. Cl. CPC A61K 31/4406; A61K 8/4926; A61K 8/606; A61K 8/675; A61K 31/4402; A61K 31/455; A61K 31/706; A61K 45/06; A61P 25/00; A61Q 19/00; A61Q 19/008; A61Q 19/08

(57) ABSTRACT

The invention relates to therapeutic uses of SARM1 agents (e.g. molecule that causes neurodegeneration and/or neuronal dysfunction in a SARM1-dependent manner) such as Vacor, metabolites, analogs, and derivatives thereof for treating and/or preventing various diseases including neurological, ophthalmological, dermatological, gastroenterological, colorectal, urological, gynaecological, rheumatological, orthopaedic, dental, and/or ear nose and throat disease. Also provided are non-therapeutic uses of SARM1 agents metabolites, analogs and derivatives thereof, for example in methods for reducing or preventing skin changes such as wrinkles, contouring, and improving oily skin.

The invention relates to the use of a SARM1 agent for use as a medicament, wherein the SARM1 agent is any molecule that causes neurodegeneration and/or neuronal dysfunction in a SARM1-dependent manner. The invention thus includes the use of SARM1 activators such as Vacor, metabolites, analogs, and derivatives thereof, as a medicament. In particular, SARM1 agents may be used in treating and/or preventing various diseases including neurological, ophthalmological, dermatological, gastroenterological, colorectal, urological, gynaecological, rheumatological, orthopaedic, dental, and/or ear nose and throat disease. The invention further relates to the use of SARM1 agents, metabolites, analogs and derivatives thereof in non-therapeutic methods for reducing or preventing skin changes such as wrinkles, contouring, and improving oily skin.

### BACKGROUND

SARM1 (Sterile alpha and Toll/interleukin-1 receptor motif-containing 1) is a central regulator of programmed axon death and is required to initiate axon self-destruction after traumatic and toxic insults to the nervous system. SARM1 is a prodegenerative enzyme having critical NAD+ (nicotinamide adenine dinucleotide) consuming activity (Essuman et al., 2017). SARM1 also consumes NADP+ and catalyses base exchange involving either cofactors (Angeletti et al., 2022; Zhao et al., 2019). In cells, SARM1 activity is regulated by the axonal survival and NAD+-synthesising enzyme NMNAT2 (nicotinamide mononucleotide adenylyltransferase 2). NMNAT2 loss in the axon results in a rise in the levels of NMNAT2 substrate NMN (nicotinamide mononucleotide), as well as NAD+ depletion, both acting to elevate the NMN:NAD+ ratio (Di Stefano et al., 2015, 2017; Gilley and Coleman, 2010; Figley et al., 2021). This rise drives SARM1 activation in axons, greatly increasing the rate of SARM1-mediated NAD+ consumption, leading to further, more rapid NAD+ depletion and accumulation of SARM1 products such as cyclic ADP-ribose (cADPR), followed by axon degeneration.

SARM1 is thus a pro-neurodegenerative enzyme which plays a central role in programmed axon death, a well-characterised and preventable/druggable mechanism of axon degeneration (Coleman and Höke, 2020; Osterloh et al., 2012). Axon loss occurs early in many neurodegenerative disorders. Preventing axon loss is therefore essential for disease-modifying therapies. In view of this, there has been much focus on the development of inhibitors of SARM1. SARM1 inhibitors are disclosed by the prior art to have a role in axonal protection. For example, Sasaki et al., (Exp Neurol. 2021 November; 345:113842) disclose that allosteric SARM1 inhibitors are useful in promote axonal protection. Similarly, U.S. Pat. No. 8,889,126 B2 disclose that SARM1 inhibitors can be used to treat axonopathy which occurs in various neurodegenerative diseases such as Parkinson's and Alzheimer's diseases as well as upon traumatic, toxic or ischemic injury to neurons. U.S. Ser. No. 11/253,503 B2 also discloses that compounds useful as inhibitors of SARM1 NADase activity can be used to treat neurodegenerative or neurological diseases. It is thus apparent that inhibitors of SARM1 have largely been exploited for their protective effects on neurons.

In contrast, SARM1 agents which cause neurodegeneration and/or neurodysfunction have not been implicated in rational medical use that is cell selective, locally administered and has a temporary effect and is reversible. For example, Wu et al., 2021. discloses injection of a SARM1 agonist into a nerve (chemical neurolysis) to treat neuropathic pain (specifically pancreatic cancer associated pain, facetogenic pain and trigeminal neuralgia). However, applications of these agents as suggested in Wu et al for treating subjects is implausible. Injecting into the pancreas will cause pancreatic degeneration and type 1 diabetes as was demonstrated by patients who ingested Vacor in the DeWitt, 1980 NEJM case series. Treatment of trigeminal neuralgia would require injection into the trigeminal ganglion which would cause widespread neurological damage and several severe unwanted side effects such as loss of salivary and tear production, sweating, permanent numbness of the whole face and a risk of paralysis on one side of the face given that the facial nerve lies close to the trigeminal ganglion. Furthermore, injecting near the spinal cord for facetogenic pain will likely cause irreversible limb paralysis through motor neuron degeneration via local leakage. By way of example, the SARM1 agent, Vacor (a pyridine derivative and a nicotinamide analog) and related compounds are well known for their bactericidal, fungicidal, herbicidal, insecticidal and rodenticidal properties. Vacor is a powerful neurotoxin associated with human peripheral and central nervous system disorders (Gallanosa et al., 1981; LeWitt, 1980) and axon degeneration in rats (Watson and Griffin, 1987). It has primarily been used as rotenticide to control numbers of the common rat, Rattus norvegicus, which poses a serious threat to the health and well-being of man. Vacor is now a disused pesticide.

US20220409648A1 which is based on data solely provided by the Wu et al., 2021 discloses Vacor and associated pyridine derivates, that activate SARM1 by directly binding of the allosteric pocket for proposed clinical uses as neuroablative agents that act in a similar manner as Botulinum toxin. It is assumed in US20220409648A1 (and Wu et al. 2021) that treatment with a ‘SARM1 activating agent’ as described therein will cause permanent neurodegeneration. However, this would not be a desirable quality for treatment of conditions that require temporary and reversible inhibition of neurotransmission such as dystonia, spasticity, neuropathic pain and cosmetic applications. Permanent neurodegeneration will lead to long-term paralysis, which may lead to swallowing and breathing difficulties and numbness of affected body parts and may lead to further disability. An effective neuroablative treatment for the aforementioned conditions ideally needs to be temporary and fully reversible, in regards that the neurons can regenerate after neuroablation, to avoid permanent disability. There is currently no prior art that suggests or indicates that ‘SARM1 activating agents’ or SARM1 agents can lead to temporary or reversible neurodegeneration. It is also disclosed in US20220409648A1 that the therapeutic mechanism of a ‘SARM1 activating agent’ is ‘neuro-specific’, and will ‘not damage non neural tissues’. It is assumed in US20220409648A1 that ‘SARM1 activating agent’ are neuro specific which underlies the use as specific neuroablative agents. US20220409648A1 also discloses that SARM1 is only present in neurons and that this also underlies the disclosed use as specific neuroablative agents. US20220409648A1 proposes that SARM1 agents have a mode of action similar to other prior art neurolytics such as phenol. US20220409648A1 also discloses that the ‘SARM1 activating agents’ may be used in any condition Botulinum toxin is used in.

US20220409648A1 makes no suggestion and provides no data indicating that SARM1 activating agents' or SARM1 agents as disclosed therein have a temporary, reversible and selective effect on cells expressing Sarm1, such as neurons, and not on surrounding cells, that do not express or express low levels of Sarm1, in the site of application, such as skin cells, muscle cells and Schwann cells.

US20220409648A1 makes no suggestions and provides no data to indicate that SARM1 agents should be administered peripherally and act in the periphery to achieve therapeutic effect and not cause disability.

SARM1 agents, such as Vacor and its metabolite VMN act on SARM1. Vacor in particular is metabolised to vacor mononucleotide (VMN) and vacor adenine dinucleotide (VAD) through a two-step conversion by nicotinamide phosphoribosyltransferase (NAMPT) and NMNAT2 (FIG. 2). VMN (an NMN analog) in turn binds and activates SARM1 causing axon degeneration. NMN (an endogenous SARM1 activator) and NAD both bind to SARM1's allosteric pocket in the ARM (armadillo/beta-catenin-like repeat) domain (the activation pocket) domain. NMN binds to the ARM domain resulting in SARM1 activation and axon degeneration. Conversely, NAD binding in the same allosteric site keeps SARM1 in an inactive state (FIG. 1). NMN and NAD thus compete for binding with opposite outcomes on SARM1 activity. An increase in NMN levels and NMN/NAD ratio leads to SARM1 activation (Figley and DiAntonio, 2020; Figley et al., 2021). Thus, when NMN levels are high, NMN binds to an allosteric pocket on the SARM1 ARM and activates SARM1, leading to increased SARM1-dependent consumption of NAD(P)+ and axon degeneration.

Neuroablative drugs currently known in the art include the use of botulinum toxin (botox), capsaicin, and phenol. These agents can be injected locally to treat, for example, neuropathic pain, and other conditions such as spasticity and dystonia. Botulinum toxin can also be used in non-therapeutic procedures to remove or prevent skin wrinkles when injected locally.

Botulinum toxin and capsaicin both work by causing a temporary disruption between the nerve and the target organ (Lim and Seet 2010; Simone et al. 1998).

In the case of Botulinum toxin the target organ is predominantly skeletal muscle for conditions characterised by dystonia, dyskinesia and spasticity and predominantly smooth muscle for gastrointestinal and urological dysmotility disorders, though injection into sphincters and into muscles for gynaecologicaly conditions can involve injection into a combination of skeletal and smooth muscle. Injection of Botulinum toxin into the muscle, prevents neurotransmitter release from presynaptic terminals and brings about a reduction in muscle tone and easing of symptoms. Botulinum toxin is also used to target secretory glands innervated by the parasympathetic nervous systems, in particular salivary glands and sweat glands. In this situation Botulinum toxin prevent acetylcholine release from presynaptic terminals and activation of target secretory glands reducing saliva or sweat production (Bach and Simman 2022; Lim and Seet 2010).

In regard to capsaicin, the target organs are the nociceptive nerve endings in the skin. Capsaicin binds TRPV1 receptors on nociceptive neurons, initially activating them but then causing inhibition and temporary degeneration, which brings about a reduction in pain transmission (Simone et al. 1998).

In addition to inhibiting neurotransmitter release between nerve and muscle Botulinum toxin can also cause denervation like changes (Thesleff, Molgó, and Tagerud 1990; Witzemann, Brenner, and Sakmann 1991) and anatomical muscle denervation which is then followed by spontaneous regeneration (Velasco et al. 2008). A number of other naturally occurring neurotoxins, such as Latrotoxin from the black widow spider, cause a temporary disruption of nerve-muscle synaptic transmission by causing degeneration of the nerve terminals which then spontaneously regenerate (Duchen, Gomez, and Queiroz 1981; Harris et al. 2000; Rigoni and Montecucco 2017). Similarly, capsaicin causes activation then inhibition and subsequent degeneration of sensory nerve terminals when applied to the skin in humans and this cellular mechanism underlies its clinical efficacy (Simone et al. 1998).

Besides the disadvantages outlined above, the effects caused by, for example, phenol are also not reversible. Phenol injections can cause permanent nervous system damage and widespread fibrosis. Injection of phenol causes severe pain and thus is only used as a treatment of last resort and when a patient has no sensation in the affected body part (Kheder et al. 2012. Pract Neurol; 12:289). Botulinum toxin, while a very effective treatment does suffer from a number of drawbacks. Firstly, there are primary and. secondary non-responders to treatment. A number of these cases are caused by the development of immunoresistance, as the immune system reacts to the bacterial proteins. Additionally, as it is a very large molecule (150 kDa) it cannot be applied via a simple transdermal patch and thus this limits its use. Furthermore, botulinum toxin has no reversal agent. In cases where the toxin diffuses far from the injection site this can lead to patients suffering from weeks to months of unintended muscle weakness, such as speech and swallowing difficulty from injections for cervical dystonia (Lim and Seet. 2010. Nat. Rev. Neurol. 6, 624; Tucker et al. 2021. Mov Dis Clin Pract; 8:541; Marion et al. 2016. Pract Neurol; 16:288). High dose capsaicin (8%) patch treatment causes severe burning pain and extensive skin erythema when applied in around a 50% of patients, often requiring co-administration of local anaesthetic or opiod analgesia (Burness and McCormack. 2016. Drugs; 76:123-134.). There is therefore a need for development of alternative therapies for conditions such as spasticity, dystonia, neuropathic pain, sphincter and autonomic dysfunction.

Therefore, it is an aim of the invention to provide new and/or improved treatments for neuropathic pain, and other conditions such as spasticity, dystonia, sphincter and autonomic dysfunction.

It is also an aim of the invention to provide new and/or improved methods of cosmetic treatment of skin changes.

### BRIEF SUMMARY OF THE DISCLOSURE

The inventors have surprisingly discovered that SARM1 agents (molecules that cause SARM1-dependent neurodegeneration and/or neuronal dysfunction) such as Vacor which result in the activation of SARM1 and have traditionally been used as neurotoxins have a first medical use. The inventors identified that SARM1 agents, for instance but not limited to activators of SARM1 can achieve selective inactivation/ablation of axons/neurons. Underlying the present invention is the finding of differential expression of SARM1 in different cell types alongside insensitivity of certain cell types to SARM1 agents establishes their uses as a cell selective therapy for the clinical conditions disclosed herein. Data in muscle cells, skin cells and Schwann cells (the axon-supporting cells in the peripheral nervous system (PNS)) showed that SARM1 is not equally expressed across different cell types and that Vacor and other SARM1 agents, for instance but not limited to activators of SARM1 are not toxic to these cells (FIGS. 3,5,6,7,8,9,15, 17, 20). The inventors identified that SARM1 protein is not present in mouse Schwann cells in vivo and in vitro (FIGS. 3 and 7). Cultured mouse Schwann cells are also completely insensitive to application of high doses of SARM1 activators such as vacor and 3-AP (FIGS. 8 and 9). Such cells did not show a reduction in cellular NAD+ levels (FIG. 8B). A further discovery was that the application of high dose Vacor to human muscle cell (myoblast) cultures had no effect on these cell types (FIG. 15). In addition, high dose Vacor to human dermal skin fibroblasts had no effect on this cell type (FIG. 17).

The finding that Schwann cell, myoblast and dermal skin fibroblast insensitivity to SARM1 agents is important as application of these agents to, for example, skin or muscle can be used in therapies and cosmetic treatments to cause selective nerve terminal degeneration in the area where they are administered, with no effect on surrounding tissues. Vacor and other SARM1 agents are as such novel neuroablative agents that specifically target neurons while not affecting other cell types where SARM1 expression is absent or low. Vacor and other SARM1 agents are thus ideal for treating and/or preventing conditions where peripheral neuroablation is desirable. Such applications include medical treatments such as pain relief and treatment of spasticity as well as cosmetic treatments to prevent wrinkles where highly desirable results can be achieved by injecting SARM1 agents into affected areas of the body.

Furthermore, SARM1 agents, for instance but not limited to activators of SARM1, operate in a different manner from other drugs used in neuroabaltive procedures such as botulinum toxin, capsaicin and phenol. Advantageously, the action of SARM1 agents is temporary and/or reversible, allowing for regeneration of axons and terminal aborizations after inducing neurodegeneration as can be seen in FIG. 18. SARM1 agents are also reversible with reversal agents such as nicotinamide making SARM1 agents particularly suitable for therapeutic and non-therapeutic treatments. Nicotinamide (NAM) competes with Vacor for NAMPT, therefore reducing Vacor conversion into the toxic metabolite VMN, and limiting SARM1 activation. It is also a precursor of NAD+, and will therefore counteract the rapid NAD+ depletion caused by Vacor-mediated SARM1 activation. A combination of these two activities is likely responsible for NAM protective effect against Vacor neurotoxicity (FIG. 2) (Loreto et al., 2021).

Further, Schwann cell, human myoblast and human dermal skin fibroblasts insensitivity to SARM1 agents is important for application of these agents to skin or muscle to act as a therapy to cause selective axon/terminal aborization/synaptic terminal degeneration. Because Schwann cells are key in promoting neural repair and regeneration (Jessen and Arthur-Farraj; Glia. 2019 March; 67 (3): 421-437), the fact they remain unaffected by high doses of SARM1 agents will permit eventual axon regeneration and target organ reinnervation after SARM1 agents have induced axon/terminal aborization/synaptic terminal degeneration. Sarm1 mRNA is also present at very low, and in one study undetectable, levels in bone marrow-derived mouse monocytes/macrophages and previous observations show no effect of Sarm1 deletion on macrophage function nor gene expression (Kim et al., 2007; Szretter et al., 2009; Uccellini et al., 2020). This indicates that SARM1 agents are unlikely to cause any toxicity to the peripheral monocyte/macrophage populations. This is important because macrophages also play an important role in supporting nerve regeneration (Cattin et al., 2015 Cell; Barrette et al., 2008. JNeurosci). Additionally, it has been shown in certain cell culture situations that Schwann cells can delay the rate of axon degeneration (Babetto et al., 2020; Mutschler et al., 2023) and thus they are likely to be able to modulate the effect of SARM1 agents on promoting targeted and selective axon/terminal aborization/synaptic terminal degeneration or dysfunction. Thus, a one off treatment with SARM1 agents will be temporary and reversible.

In contrast to the prior art, it has been surprisingly found that ‘SARM1 activating agents’ or SARM1 agents can lead to temporary or reversible neurodegeneration while also allowing for neuro-regeneration after administration (FIG. 18). This finding for the first time makes it feasible that SARM1 agents could be used to treat conditions such as spasticity, dystonia, peripheral neuropathic pain, autonomic dysfunction, and be used for cosmetic applications without causing harm or permanent disability. This is due to the fact that permanent action of SARM1 agents causing permanent and or long lasting neurodegeneration would be not be a desirable quality for treatment of conditions that require temporary and reversible inhibition of peripheral neurotransmission such as dystonia, spasticity, peripheral neuropathic pain, autonomic dysfunction and cosmetic applications. Permanent neurodegeneration may lead to long-term paralysis, which may include loss of limb muscle function, swallowing and breathing difficulties, risk of aspiration and death, loss of vision, bowel and bladder incontinence, impotence, and numbness of affected body parts and lead to further disability and or require corrective treatments and or surgery. An effective neuroablative treatment for the aforementioned conditions ideally needs to be temporary and fully reversible, in regards that the neurons can regenerate after neuroablation, to avoid permanent disability.

In addition, the inventors have unexpectedly and surprisingly found that Schwann cells are insensitive to SARM1 agents (FIGS. 8 and 9). This is contrast to the prior art which indicates that SARM1 agents are neuro-specific’, and do ‘not damage non neural tissues’. This definition neglects that for a ‘SARM1 activating agent’ or SARM1 agents to be used as a therapy in the aforementioned conditions it must not harm non-neuronal cells contained within neural tissue. In particular, Schwann cells, which wrap around and/or are closely associated with axons, in nerve roots and nerve trunk, in the case of myelinating and non-myelinating Schwann cells, and Schwann cells associated with the terminal aborizations of neurons within neural tissue, in the case of terminal/perisynaptic and nociceptive Schwann cells, are key to promoting neural repair and regeneration, through their transformation into injury activated and/or repair Schwann cells (Jessen and Arthur-Farraj; Glia. 2019 March; 67 (3): 421-437). The insensitivity of Schwann cells to ‘SARM1 activating agent’ or SARM1 agents is key to the temporary and reversible nature of the treatments proposed.

It is unknown in the prior art whether SARM1 is present and functional in myelinating glia in the CNS or PNS and also in non-neuronal cells of the PNS and cells of target organs (ie. skin and muscle) that the PNS innervates. The inventors have surprisingly found that SARM1 protein is present and functional in central nervous system myelinating glia, oligodendrocytes but almost entirely absent or present in functionally negligible levels in Schwann cells in the peripheral nervous system and in Schwann cells after nerve injury (FIG. 3,5,7,8,9, 20). The contrasting findings of the levels of SARM1 between the central and peripheral nervous system glia underlies the herein disclosed use of SARM1 agents as a temporary and reversible method of peripheral neuroablation in all of the aforementioned conditions. In further advance of this, the inventors have also surprisingly shown that other peripheral cells, human myoblasts and human dermal skin fibroblasts are insensitive to high concentrations of SARM1 agents, strongly indicating that they too contain very low levels of functional SARM1 (FIGS. 15 and 17).

Furthermore, the inventors have found that for the treatment of some conditions, SARM1 agents may be peripherally administered to be therapeutically effective and not cause harm. This is in contrast to the prior art which includes treatment of conditions such as post amputation pain which is a central pain condition and would require administration of a SARM1 agents in or near the spinal cord or brain which would cause severe disability or even death. The discovery that CNS myelinating glia contain functional SARM1 by the inventors (FIGS. 3, 5 and 7) adds significant further weight to the implausibility of the proposal to use SARM1 agents as treatments for conditions that would require administration of them in or near the CNS as they would not be cell selective, targeting multiple cell types, causing widespread damage and lasting disability.

In a first aspect of the invention there is provided, a SARM1 agent for use as a medicament.

In a first aspect of the invention there is provided, a SARM1 agent for use in a method of treating and/or preventing neurological, ophthalmological, dermatological, gastroenterological, colorectal, urological, gynaecological, rheumatological, orthopaedic, dental, and/or ear nose and throat disease.

In certain embodiments, the disease is defined by autonomic dysfunction, neuropathic pain, dystonia, spasticity, dyskinesia, sphincter dysfunction, genitourinary dysfunction and/or a skin condition.

In certain embodiments, the autonomic dysfunction comprises excessive sweating (hyperhidrosis). In certain embodiments, the autonomic dysfunction comprises excessive saliva production (sialorrhoea).

In certain embodiments, the autonomic dysfunction comprises autonomic dysfunction caused by inherited and/or genetic neurological diseases. In certain embodiments, the inherited and/or genetic neurological diseases comprise hereditary autonomic neuropathy, and/or sensory autonomic neuropathy.

In certain embodiments, the autonomic dysfunction comprises autonomic dysfunction caused by degenerative neurological diseases. In certain embodiments, the degenerative neurological diseases comprise amyotrophic lateral sclerosis, Parkinson's disease, parkinsonism, multiple system atrophy, cerebral palsy and/or degenerative neurological diseases due to injury.

In certain embodiments, the autonomic dysfunction comprises autonomic dysfunction caused by disease of the autonomic nervous system from an acquired disorder (secondary dysautonomia). In certain embodiments, disease of the autonomic nervous system from an acquired disorder (secondary dysautonomia) comprise infections (COVID and Long COVID), autoimmune conditions, postural orthostatic tacycardia syndrome (POTS), and/or endocrine conditions. In certain embodiments, endocrine conditions comprise diabetes and/or thyroid dysfunction.

In certain embodiments, the autonomic dysfunction comprises autonomic dysfunction caused by small fibre neuropathy.

In certain embodiments, the autonomic dysfunction comprises autonomic dysfunction caused by connective tissue disease.

In certain embodiments, the autonomic dysfunction comprises autonomic dysfunction caused by peripheral nerve injuries.

In certain embodiments, the autonomic dysfunction comprises autonomic dysfunction caused by surgical repair of peripheral nerve injuries.

In certain embodiments, the autonomic dysfunction comprises autonomic dysfunction caused by medications.

In certain embodiments, the autonomic dysfunction comprises autonomic dysfunction caused by idiopathic autonomic dysfunction.

In certain embodiments, neuropathic pain comprises peripheral neuropathic pain; chronic back pain; cancer related pain; myofascial pain; fibromyalgia; complex regional pain disorder; chronic pelvic pain; dyspareunia; vulvodynia; and/or painful bladder syndrome (interstitial cystitis). In certain embodiments, neuropathic pain comprises peripheral neuropathic pain.

In certain embodiments, neuropathic pain comprises neuropathic pain caused by diabetic neuropathy neuralgia post peripheral nerve injuries; infective and post-infective neuralgias; metabolic neuropathies; small fibre neuropathy; endocrine conditions; genetic neuropathies; paraproteinaemic neuropathies; paraneoplastic neuropathies; neoplastic neuropathies; vasculitis; auto-immune, inflammatory and demyelinating neuropathies; idiopathic neuropathies, peripheral nerve injuries and/or surgical repair of peripheral nerve injuries.

In certain embodiments, dystonia comprises: primary dystonia; and/or dystonia plus conditions.

In certain embodiments, dystonia comprises dystonia occurring with neurodegenerative diseases.

In certain embodiments, dystonia comprises secondary dystonia occurring due to: central nervous system (CNS) trauma, congenital malformation, genetic and chromosomal disorders, infection, neoplasm, ischaemic; haemorrhagic stroke, inflammation, demyelination, drugs, toxins, and/or metabolic disturbance;

In certain embodiments, spasticity comprises upper limb spasticity, lower limb spasticity; bladder spasticity/neurogenic bladder; spastic disorders of the oesophagus.

In certain embodiments, spasticity comprises spasticity caused by: demyelinating conditions; congenital malformation; chromosomal disorders; genetic conditions; CNS infections; neoplasm; drugs; toxins; metabolic disturbance; paraneoplastic disorders; post-infections conditions; autoimmune conditions; endocrine conditions, peripheral nerve injuries and/or surgical repair of peripheral nerve injuries;

In certain embodiments, dyskinesia comprises: hemifacial spasm and facial nerve synkinesis; and/or palatal myoclonus/tremor.

In certain embodiments, dyskinesia comprises dyskinesia caused by: tic disorders; tremor; myokymia, trauma, infection, autoimmune conditions; neuromyotonia caused by autoimmune and/or hereditary conditions;

In certain embodiments, sphincter dysfunction is caused by: dysfunction of bodily sphincters; pyloric sphincter; sphincter of Oddi dysfunction leading to gastrointestinal dysmotility; pancreatic sphincter; urethral sphincter; anal fissure; and/or Hirshsprungs disease.

In certain embodiments, genitourinary dysfunction comprises: vaginismus; and/or voiding dysfunction.

In certain embodiments skin condition comprises: an ulcer; acne; and/or raynauds phenomenon/disease.

In certain embodiments orthopaedic disease comprises: sports injuries; posttraumatic elbow stiffness; club foot; and/or piriformis syndrome.

In certain embodiments, dental disease comprises: odontalgia; and/or root canal treatment.

Provided in a third aspect of the invention is a non-therapeutic method of:
(i) removing and/or preventing skin changes;
(ii) contouring; and/or
(iii) improving oily skin;
the method comprising administering a SARM1 agent.

In certain embodiments, skin changes comprise one or more of skin wrinkles, frown/glabellar lines, forehead lines, lateral canthal lines/crow's feet, horizontal nasal bridge lines, neck bands, platysmal neck lines, smoker's lines, bunny lines, top lip lines, ptosis of the lateral commissure, marionette lines, gummy smile, chin dimples, peau d'orange chin, pitted skin, eye brow sagging, eyelid sagging, lip sagging, square jaw, and a downturned nasal tip.

In certain embodiments contouring comprises (i) shaping lip, cheek, jaw, and/or temple; and/or (ii) eyebrow contour.

In certain embodiments of any aspect of the invention, the SARM1 agent causes SARM1 dependent neurodegeneration and/or neuronal dysfunction. In certain embodiments of any aspect of the invention, the SARM1 agent activates SARM1 and neurodegeneration and/or neuronal dysfunction.

In certain embodiments of any aspect of the invention, the SARM1 agent activates SARM1 by binding to SARM1. In certain embodiments of any aspect of the invention, the SARM1 agent increase intracellular levels of NMN. In certain embodiments, the SARM1 agent decreases intracellular NAD levels. In certain embodiments the SARM1 agent increases an intracellular NMN/NAD ratio.

In certain embodiments, the neurodegeneration comprises axonal, terminal aborization, or synaptic terminal degeneration and/or neurolytic death, optionally wherein the axonal degeneration, terminal aborization and/or synaptic terminal and/or neurolytic death is reversible. In certain embodiments, the neurodegeneration is temporary. In certain embodiments, the neurodegeneration allows neural regeneration to occur after the neurodegeneration. That is to say that after SARM1 agent induced neurodegeneration, the neuronal cells may be able to regenerate. For example, neurons can regenerate their axons following degeneration caused by administration of a SARM1 agent as described herein. For example, local administration.

In certain embodiments, the neurodegeneration and/or neuronal dysfunction is reversible by a reversal agent.

In certain embodiments, neurodegeneration and/or neuronal dysfunction:
(ii) occurs in cells expressing SARM1, optionally wherein the cells expressing SARM1 comprise neurons; and
(ii) does not occur in cells lacking SARM1, or expressing functionally insignificant amounts of SARM1 protein, optionally wherein the cells lacking SARM1 or expressing functionally insignificant amounts of SARM1, comprise Schwann cells (myelinating, non-myelinating/Remak, perisynaptic/terminal, nociceptive nor injury activated/Repair Schwann cells); muscle cells, skin cells (such as dermal fibroblasts), HEK293T cells, Hela cells, SK mel 2 cells, SK mel 5 cells, SK mel 28 cells, CAPAN cells, JURKAT cells, C6 cells, HL-60 cells, LP-1 cells, U937 cells, and/or MEWO cells, monocytes, macrophages, and/or RAW264.7 cells (Uccellini et al., 2020).

In certain embodiments, the SARM1 agent is selected from vacor; vacor mononucleotide (VMN); 3-acetylpyridine (3-AP); 3-acetylpyridine mononucleotide (3-APMN); 2-aminopyridine (2-AnP); 2-AnP mononucleotide (2-AnPMN); nicotinamide mononucleotide (NMN), sulfo-ara-F-NMN (CZ-48), sulfo-ara-F-VMN; sulfo-ara-F-3-APMN; sulfo-ara-F-2-AnPMN, S-NMN, ara-F-NMN (CZ-17); pyridines and molecules containing pyridine rings; vacor riboside (VR), vacor riboside (VR) analogs, nicotinamide (NAM) analogs; nicotinamide riboside (NR), nicotinamide riboside (NR) analogs; nicotinic acid (NA) analogs; nicotinic acid riboside (NaR) analogs, nicotinic acid mononucleotide (NaMN) analogs, molecules that increase intracellular levels of NMN, decrease NAD levels and/or increase NMN/NAD ratio resulting in SARM1 activation; NMNAT1-2-3 inhibitors or molecules that reduce NMNAT1-2-3 levels; and/or metabolites, analogs and derivatives thereof.

In certain embodiments, the analogs and derivatives increase permeability and/or solubility of the SARM1 agent.

In certain embodiments, the SARM1 agent is administered locally, optionally by:
(i) a transdermal patch or
(ii) injection into tissue such as (a) the skin or muscle, optionally intravesical injection into detrusor muscle/urethral sphincter, with or without ultrasound, radiographic and/or electromyographic guidance, (b) a neuroma, optionally for peripheral nerve regeneration and surgical nerve repair, and/or (c) a root canal, optionally for use in dental surgery;
(iii) endoscopic injection such as into the oesophogeal sphincter, pyloric sphincter, sphincter of Oddi; cystoscopic injection into the urethral sphincter;
(iv) injection into salivary glands such as sublingual and submandibular glands; or
(v) topical administration.

In certain embodiments, two or more SARM1 agents as described herein are co-administered, optionally wherein an additional agent such as a reversal agent and/or an analgesic/anaxesthetic is administered.

In a further aspect there is provided a composition comprising a SARM1 agent as described herein; optionally wherein the composition is a pharmaceutical composition and further comprises at least one pharmaceutically acceptable excipient, diluent, and/or carrier.

In certain embodiments, the composition as described herein is, for use in any methods as described herein.

In certain embodiments, the composition is a topical composition; optionally wherein the composition is a cream or ointment.

In certain embodiments of the third aspect, the SARM1 agent is comprised in a composition as described herein.

In a further aspect there is provided a syringe comprising a SARM1 agent or a composition as described herein.

In a further aspect there is provided a transdermal patch comprising a SARM1 agent or a composition as described herein.

In a further aspect there is provided a kit of parts comprising:
a. a SARM1 agent or composition as described herein; and
b. an applicator configured for administration of the SARM1 agent.

In certain embodiments the applicator comprises a transdermal patch as described herein.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### BRIEF DESCRIPTION OF THE DRAWINGS

Embodiments of the invention are further described hereinafter with reference to the accompanying drawings, in which:

FIG. 1 shows a schematic of (A) the programmed axon death pathway, and (B) the mechanism of SARM1 activation.

FIG. 2 shows a schematic (A) of how SARM1 activation leads to axon degeneration and (B) how reversal of SARM1 activation by nicotinamide is achieved.

FIG. 3: shows detection of SARM1 protein associated with CNS but not PNS myelinating glia A) Representative immunofluorescence images of transverse sections of Wt mouse tibial nerves to show co-localisation of SARM1 and NF-200 (neurofilament light chain) in axons. Zoomed area is represented by the white bounding box. Scale bars 10 μm. B) Representative immunofluorescence images of transverse sections of Wt mouse optic nerves to show expression of SARM1 in TUJ1 (Anti-Beta III Tubulin) positive axons. Zoomed area is represented by the white bounding box. Scale bars 10 μm. C) Representative immunofluorescence images of transverse sections of Wt mouse DRGs to show co-localisation of SARM1 and NF-200 positive neurons. Scale bar 25 μm. D) Representative immunofluorescence images of transverse sections of Wt and Sarm1 KO mouse tibial nerves showing SARM1 and SOX10 expression. White arrowheads show co-localisation of DAPI and SOX10, but no expression of SARM1. Zoomed areas are represented by the white bounding box. Scale bar 10 μm. E) Representative immunofluorescence images of transverse sections of Wt and Sarm1 KO mouse DRGs showing SARM1 and SOX10 expression. White arrowheads point to SOX10 positive satellite glia and DAPI, with no colocalization of SARM1. Scale bar 10 μm. F) Representative immunofluorescence images of transverse sections of Wt and Sarm1 KO mouse optic nerves showing SARM1 and SOX10 expression. White arrowheads point to SOX10 positive oligodendrocytes and DAPI, with perinuclear SARM1 staining. Scale bar 10 μm. All experiments n=3 (3 animals, 2 nerves per animal, 5 sections per nerve).

FIG. 4: shows no first layer SARM1 antibody controls A) Representative immunofluorescence images of transverse sections of Wt and Sarm1 KO mouse dorsal root ganglia (DRGs) to show lack of SARM1 signal in the absence of addition of primary antibody. NF-200 (neurofilament light chain) to show positive neurons. Scale bar 25 μm. B) Representative immunofluorescence images of transverse sections of Wt and Sarm1 KO mouse dorsal root ganglia (DRGs) to show lack of SARM1 signal in the KO sample in the presence the primary antibody. The Wt panel shows co-localisation of NF-200 positive neurons with SARM1. Scale bar 25 μm.

FIG. 5: shows sarm1 expression in zebrafish larvae posterior lateral line nerve (PLLn) and spinal cord. A) MAX projection, lateral view of PLLn of a 5dpf larva demonstrating HCR in situ labelling for sox 10 and sarm1 mRNA and DAPI staining to mark nuclei. B) MAX projection, lateral view of spinal cord of a 5dpf larva demonstrating HCR in situ labelling for sox 10 and sarm1 mRNA and DAPI staining to mark nuclei. C) Single confocal z plane, 616 nm optical thickness, lateral view of PLLn of a 5dpf larva. Arrowheads mark nuclei, labelled with DAPI, positive for sox10 and sarm1 mRNA. For all images, scale bar 25 μm. All experiments n=7 biological replicates.

FIG. 6 shows sarm1 expression in zebrafish larvae hypothalamus and retina. A) MAX projection, lateral view of the PLLn of a 5dpf larva with no probes (no first layer control) to sox10 nor sarm1 applied. Images show DAPI, and hairpins tagged to Alexa647 and Alexa546. Scale bar 25 μm. B) MAX projection, lateral view of the spinal cord of a 5dpf larva with no antisense probes to sox10 nor sarm1 applied. Images show DAPI, and hairpins tagged to Alexa647 and Alexa546. Scale bar 25 μm. C) MAX projection, lateral view of the hypothalamus of a 5dpf larva showing sox10 (magenta) and sarm1 (green) mRNA expression. Nuclei labelled with DAPI (grey). Scale bar 25 μm. D) MAX projection, lateral view of retina of a 5dpf larva showing sox 10 and sarm1 mRNA expression. Nuclei labelled with DAPI. Scale bar 25 μm.

FIG. 7 shows Sarm1 mRNA but not SARM1 protein is detectable in mouse Schwann cells. A) RT PCR of cDNA from Wt cultured mouse Schwann cells, Sarm1 KO mouse Schwann cells and Wt and Sarm1 KO embryonic (E14.5) cultured mouse dorsal root ganglion neurons (n=4:4 separate cultures prepared from 4 separate timed matings). B) qPCR analysis of Sarm1 expression in cultured mouse Wt and Sarm1 KO DRGs and Wt and Sarm1 KO Schwann cells (SC, n=4 (4 biological replicates: 4 sciatic nerves from 2 animals pooled per biological replicate); **p<0.01). C) Freshly plated Wt and Sarm1 KO, P2 dissociated mouse Schwann cells both have undetectable levels of SARM1 protein. Cultures labelled for DAPI and SOX10 (n=3: two-four mice each from three litters producing three separate cultures). Scale bar 50 μm. D) Cultured rat oligodendrocytes (SOX10 positive) express SARM1 protein using a commercial polyclonal anti-SARM1 antibody. Zoomed in area (indicated by dashed box). Arrowheads indicate SOX10 positive SARM1 positive cells. Scale bars 50 μm.

FIG. 8 shows Cultured oligodendrocytes, but not Schwann cells, are sensitive to vacor induced cell death. A) Dissociated and freshly plated P2 mouse Schwann cells cultured in DMSO or 100 μM vacor for 72 hours show no cell death, judged by propidium iodide (P.I) and DAPI nuclear staining and SOX10 immunocytochemistry (n=3: two-four mice, each from three litters producing three separate cultures). Scale bar 25 μm. B) Intracellular NAD+ levels do not change in Schwann cells when treated for 72 hours with either 250 mM 3AP or 100 μM vacor compared to DMSO control conditions. C) Rat oligodendrocytes cultured in DMSO or 100 M vacor for 72 hours demonstrate substantial cell death judged by DAPI nuclear staining and SOX10 immunocytochemistry (n=3: one rat from three litters producing three separate cultures). Scale bar 50 μm. D) Quantification of SOX10 positive oligodendrocyte cell survival in response to 100 μM vacor treatment compared to DMSO control cultures. E) Cultured rat oligodendrocytes (SOX10 positive) express SARM1 protein using a polyclonal anti-SARM1 antibody generated by Yi-Ping Hsueh. Zoomed in area (indicated by dashed box). Arrowheads indicate SOX10 positive SARM1 positive cells. Scale bars 50 μm.

FIG. 9 shows cultured Schwann cells are insensitive to 3-AP induced cell death. A) Dissociated and freshly plated P2 mouse Schwann cells cultured in water or 250 μM 3-AP for 72 hours show no cell death, judged by propidium iodide (P.I) and DAPI nuclear staining and SOX10 immunocytochemistry (n=3: two-four mice, each from three litters producing three separate cultures). Scale bar 25 μm. B) Intracellular NAD⁺ levels decrease in HEK293 cells when treated for 72 hours with 100 μM vacor.

FIG. 10 shows Axon degeneration after PLLn laser axotomy is substantially delayed in absence of functional Sarm1 in zebrafish larvae. A) Schematic of overview of 2-photon laser axotomy of the PLLn (green) in larval zebrafish. B) wt and sarm1^{SA11193/SA11193} (sarm1 mutant) fish were injected at the one cell zygote stage with neuroD: tdtomato DNA constructs and 2-photon laser axotomy was performed in both genotypes at 4dpf. While wt PLLn axons start to degenerate around three hours and are completely degenerated at six. hours post axotomy, PLLn axons in sarm1 mutant larvae remain intact at least up until 26 hours post imaging (imaging stopped due to UK home office regulations) (n=5). Scale bar 100 μm.

FIG. 11 shows myelination of PLLn and spinal cord is unaffected by absence of functional Sarm1 in zebrafish. A) Max projection of the lateral view of wild-type (wt) Tg[mbp: EGFP-CAAX] and sarm1^{SA11193/SA11193} (sarm1 mutant, mut) Tg[mbp: EGFP-CAAX] 5dpf larva. Arrowhead (white) delineates the dorsal spinal cord; Arrow (white) marks the ventral spinal cord; Arrow (grey) pinpoints the PLLn. Scale bar 100 μm. B) Zoomed in lateral view of spinal cord and PLLn (area indicated by boxes in A). Arrowhead (white) delineates the dorsal spinal cord; Arrow (white) marks the ventral spinal cord; Arrow (grey). Scale bar 25 μm. C) Relative intensity of GFP in PLLn of Tg[mbp: EGFP-CAAX] wt and sarm1 mutant (mut) (n=9; p=08633). D) Relative intensity of GFP in the dorsal and ventral spinal cord combined of Tg[mbp: EGFP-CAAX] wt and sarm1 mutant (mut) (n=9; p=08633). E) Relative intensity of GFP in the dorsal spinal cord of Tg[mbp: EGFP-CAAX] wt and sarm1 mutant (mut) (n=9; p=09314). F) Relative intensity of GFP in the ventral spinal cord of Tg[mbp: EGFP-CAAX] wt and sarm1 mutant (mut) (n=9; p=08633). G) MAX projection, lateral view of PLLn of wt and sarm1 mutant (mutant) 5dpf larvae showing mbp mRNA expression. Nuclei labelled with DAPI (grey). Scale bar 25 μm. Heatmap of intensity of nuclear mbp mRNA expression (mbp*) in PLLn of wt and sarm1 mutant (mut) 5dpf larvae. Dark nuclei represent cells with high nuclear expression of mbp. Scale bar 20 μm H) Quantification of mbp mRNA signal intensity/um in PLLn of wt and sarm1 mutant (mut) 5dpf larvae (WT n=6; MUT n=7; p=0.8357). J) Quantification of DAPI signal intensity/um in PLLn of wt and sarm1 mutant (mut) 5dpf larvae (WT n=6; MUT n=7; p=0.7308). K) MAX projection, lateral view of spinal cord of wt and sarm1 mutant (mut) 5dpf larvae showing mbp. mRNA expression. Nuclei labelled with DAPI. Scale bar 25 μm. L) Quantification of mbp mRNA signal intensity/um in dorsal and ventral spinal cord combined of wt and sarm1 mutant (mut) 5dpf larvae (wt n=6; mut n=7; p=0.4452). M) Quantification of DAPI signal intensity/um in spinal cord of wt and sarm1 mutant (mut) 5dpf larvae (wt n=6; mut n=7; p=0.7308)

FIG. 12 shows PNS myelination and myelin maintenance are normal in Sarm1 null mice A) Representative electron micrographs taken at ×3000 magnification from Wt and Sarm1 knockout (KO) tibial nerves at postnatal day 2 (P2). Scale bar 5 μm. B) Per nerve profile, the total number of axons>1.5 mm quantified are not significantly different in Wt and Sarm1 KO nerves (n=5; p=0.3095). C) The number of myelinated axons quantified per nerve profile are similar in both Wt and Sarm1 KO nerves (n=5; p=0.3095). D) The number of unmyelinated axons>1.5 μm is not significantly different in Wt and Sarm1 KO nerves (n=5; p=0.1508). E) Per nerve profile, the number of unmyelinated axons>1.5 μm in a ratio 1:1 is slightly higher in Sarm1 KO nerves compared to Wt; however, this does not reach significance (n=5; p=0.0794). F) The percentage of myelinated axons versus non-myelinated axons present in Wt and Sarm1 KO nerves is not significantly different (n=5; p=0.1508). G) The number of Schwann cell nuclei quantified per nerve profile is not significantly different between Wt and Sarm1 KO nerves (n=5; p=0.5). H) The mean total nerve area of Wt (29131 mm²) and Sarm1 KO (35291 mm²) nerves is not significantly different (n=5; p=0.1508). J) Representative electron micrographs taken at ×3000 magnification from adult Wt and Sarm1 KO tibial nerves at P60. There are no ultrastructural differences in the tibial nerves of adult Sarm1 KO compared to those of WT tibial nerves. Scale bar 5 μm. K) Per nerve profile, the total number of axons>1.5 μm quantified is similar in Wt and Sarm1 KO nerves (n=4; p=0.3429). L) The number of myelinated axons present in Wt and Sarm1 KO nerves is not significantly different (n=4; p=0.3429). M) Per nerve profile, the number of unmyelinated axons>1.5 μm is not significantly different in Wt and Sarm1 KO nerves (n=4; p=0.3429). N) The number of Schwann cell nuclei is similar in both Wt and Sarm1 KO nerves (n=4; p=0.4857). P). Myelin sheath thickness as depicted by g-ratios is similar in both Wt and Sarm1 KO nerves (n=4; p=0.4857). Q) The percentage of myelinated versus non-myelinated axons present in both Wt and Sarm1 KO nerves is not different (n=4; p=0.3429). R) Per nerve profile, the total nerve area of both Wt (156715 μm²) and Sarm1 KO (154927 μm²) nerves is very similar (n=4; p=0.8857).

FIG. 13 shows Myelin gene expression is normal in Sarm1 null peripheral nerves A) Relative mRNA expression for chemokine, Schwann cell injury and myelin genes in the P60 uninjured tibial nerve of Wt and Sarm1 KO mice. All fold change values normalized to uninjured Wt tibial nerve (n=4; *p<0.05, **p<0.01). B) Representative Western blot image of tibial nerve protein extracts from P60 Wt and Sarm1 KO mice. The image shows no difference in levels of EGR2 and JUN, between Wt and Sarm1 KO nerves. C) There is no significant difference in EGR2 levels in Wt and Sarm1 KO nerves (n=7; p=0.9118). EGR2 protein levels are normalized to the levels in Wt nerves, which are set as 1. D) There is no significant difference in JUN levels in Wt and Sarm1 KO nerves (n=7; p=0.5787). The quantifications are normalized to the levels in Wt nerves, which are set as 1. E) Representative Western blot image showing no difference in levels of Myelin protein zero (MPZ) and Myelin basic protein (MBP), between P60 Wt and Sarm1 KO nerves. F) There is no significant difference in MPZ levels in Wt and Sarm1 KO nerves (WT n=6; KO n=5; p=0.3176). The quantifications are normalized to the levels in Wt nerves, which are set as 1. G) There is no significant difference in MBP levels in Wt and Sarm1 KO nerves (n=7; p>0.9999). The quantifications are normalized to the levels in Wt nerves, which are set as 1.

FIG. 14 shows Optic nerve myelination is normal in Sarm1 null mice A) Representative electron micrographs taken at ×3000 (left hand side panels) and ×12000 (right hand side panels) magnification from adult Wt and Sarm1 KO optic nerves at postnatal day 60 (P60). There are no ultrastructural differences in the optic nerves of Sarm1 KO compared to those of Wt animals. Scale bar 5 μm (×3000) and 2 μm (×12000). B) The total number of axons quantified per optic nerve profile are not significantly different between Wt and Sarm1 KO (n=6 WT; n=5 KO; p=0.9307). C) The number of myelinated axons quantified in Wt and Sarm1 KO optic nerves is similar (n=6 WT; n=5 KO; p=0.4286). D) The percentage of myelinated axons quantified in Wt and Sarm1 KO optic nerves is not significantly different (n=6 WT; n=5 KO; p=0.0736). E) Per nerve profile, the total nerve area of both Wt (53686 μm²) and Sarm1 KO (56979 μm²) optic nerves were not significantly different (n=6 WT; n=5 KO; p=0.1255). F) Relative mRNA expression for chemokine, oligodendrocyte and myelin genes in the P60 uninjured optic nerve of Wt and Sarm1 KO mice. All fold change values normalized to uninjured WT optic nerve (n=4; *p<0.05, **p<0.01). G) Representative western blot image of optic nerve protein extracts shows no difference in levels of MBP, between Wt and Sarm1 KO nerves. H) There is no significant difference in MBP expression between Wt and Sarm1 KO optic nerves (n=6; p=0.3939). The quantifications are normalized to the levels in uninjured Wt nerves, which are set as 1.

FIG. 15 shows that cultured human myoblasts are resistant to vacor-induced cell death. Representative images of cultured human myoblasts treated for 24 h with DMSO or vacor. Propidium iodide (P.I.) staining shows that no cell death occurs even in the presence of vacor.

FIG. 16 shows A) NMN-interacting residues in Drosophila and human SARM1, based on the dSARM1ARM crystal structure (from Figley et al. Neuron 2021). B) VMN-interacting residues in Drosophila and human SARM1 (in brackets), based on the dSARM1ARM crystal structure (from Loreto et al. eLife 2021). C) Interactions between ARM domain and NMN (stick representation) of human SARM1 in ARMSAM: NMN. Polar contacts are shown as yellow dashed lines (from Shi et al. Mol Cell 2022).

FIG. 17 shows cultured human dermal skin fibroblasts are resistant to vacor-induced cell death. Representative images of cultured human dermal skin fibroblasts treated for 72 h with DMSO or vacor. Propidium iodide (P.I.) staining shows that no cell death occurs in the presence of high concentration Vacor.

FIG. 18 shows Human foetal DRG axons regenerate following localised Vacor treatment. (A) Human foetal DRGs were cultured in microfluidic chambers to allow localised treatment of axons (green compartment). (B) DRG axons degenerate 24 h after addition of 25 μM Vacor in the axonal compartment, while untreated neuronal somas remain healthy. 24 h after Vacor treatment, when axons have already degenerated, Vacor was washed out and fresh media was added to the axonal compartment. DRG axons started to regrow and fully repopulated the axonal compartment 120 h after Vacor removal. This indicates that localised Vacor treatment is temporary and/or reversible and/or allows neuronal regeneration.

FIG. 19 shows highly expanded/passaged mouse Schwann cells in culture are sensitive to SARM1 agents, vacor and 3-AP. A) Lower power magnification (x10) image montages of Phase contrast (Phase) microscopy and Propidium lodide (P.I.) staining across individual cultures of mouse Schwann cells that have been passaged three times in proliferative medium prior to being treating with 100 μM vacor or DMSO as a control for 72 hours. Scale bar 200 μm. B) High power magnification (x40) images of Phase contrast (Phase) microscopy and Propidium lodide (P.I.) staining of individual cultures of mouse Schwann cells that have been passaged three times in proliferative medium prior to being treating with 100 μM vacor or DMSO as a control for 72 hours. Arrowheads show P.I. staining colocalises with dying or dead cells identified by phase microscopy (Merge). Scale bar 100 μm. In both A) and B) there is a substantial increase in P.I. staining in vacor treated cultures indicating an increase cell death. C) Lower power magnification (x10) image montages of Phase contrast (Phase) microscopy and Propidium lodide (P.I.) staining across individual cultures of mouse Schwann cells that have been passaged three times in proliferative medium prior to being treating with 250 μM 3-AP or water as a control for 72 hours. Scale bar 200 μm. D) High power magnification (x40) images of Phase contrast (Phase) microscopy and Propidium lodide (P.I.) staining of individual cultures of mouse Schwann cells that have been passaged three times in proliferative medium prior to being treating with 250 μM 3-AP or water as a control for 72 hours. Arrowheads show P.I. staining colocalises with dying or dead cells identified by phase microscopy (Merge). Scale bar 100 μm. In both C) and D) here is a substantial increase in P.I. staining in 3-AP treated cultures indicating an increase cell death.

FIG. 20 shows Schwann cells do not contain detectable SARM1 after nerve injury. Immunohistochemistry for SARM1, SOX10 (labelling Schwann cells), Neurofilament light chain (NF) and DAPI nuclear stain in uninjured and injured tibial nerves of C57BL/6J 6-8-week-old mice. Injury (transection) to the sciatic nerve was performed at the sciatic notch and distal tibial nerves were harvested, at least 1-1.5 cm from the site of lesion, fixed and processed for immunocytochemistry. Distal tibial nerves were taken and analysed at 24 and 48 hours and 7 days after injury. SARM1 staining does not colocalise with SOX10 at any point after nerve injury indicating that Schwann cells do not contain detectable SARM1 protein in vivo after nerve injury.

The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Various aspects of the invention are described in further detail below.

### DETAILED DESCRIPTION

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

### SARM 1 Agent

A SARM1 agent may be a molecule that causes SARM1-dependent neurodegeneration and/or neuronal dysfunction.

SARM1 is a multi-functional, prodegenerative enzyme with Nicotinamide adenine dinucleotide phosphate (NAD(P))-consuming activity. SARM1 possesses NAD(P) hydrolysis, cyclization and base exchange activity. In physiological conditions, SARM1 has a basal NAD(P)ase activity which slowly consumes NAD and NADP, with production of Nicotinamide, cyclic ADP-ribose (cADPR), ADP-ribose (ADPR), phospho-cyclic ADP-ribose (cADPRP), phospho ADP-ribose (ADPRP). In physiological conditions, SARM1 basal activity does not lead to neurodegeneration and/or neuronal dysfunction. In the presence of a toxic stimuli or after administration of a SARM1 agent, or mutations of the SARM1 coding sequence, SARM1 enzymatic activity increases meaning that the rate of NAD(P) consumption increases, and the levels of SARM1 products increases (e.g. Nicotinamide, cADPR, ADPR, cADPRP, ADPRP). A SARM1 agent may act by activating SARM1 and increase NAD(P) consumption (Angeletti et al 2022, iScience). Methods to determine when SARM1 has been activated and when SARM1-neurodegeneration has occurred are described herein.

In general, a direct SARM1 agent may bind directly to SARM1 activating it, and causing SARM1-dependent neurodegeneration and/or neuronal dysfunction. An indirect SARM1 agent may activate SARM1 and cause SARM1-dependent neurodegeneration and/or neuronal dysfunction via an alternative mechanism (not direct binding to SARM1). For example, via one or more binding partners or regulate the activity of SARM1 via other molecules. A SARM1 agent may also be a molecule that does not activate SARM1, does not bind to SARM1 but still causes SARM1-dependent neurodegeneration and/or neuronal dysfunction via an alternative mechanism.

Examples for each category are provided below.

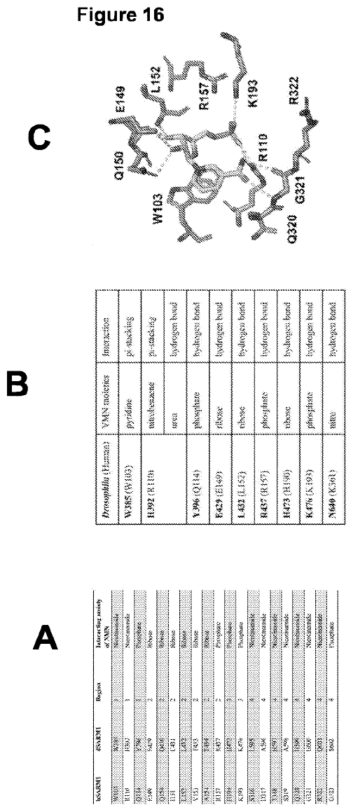

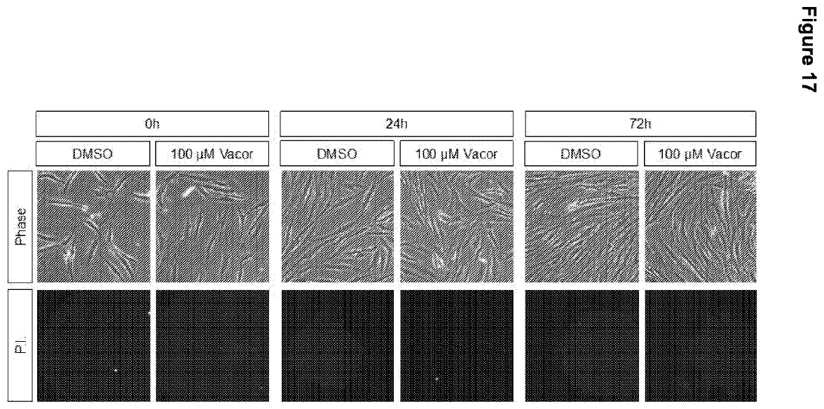

A SARM1 agent may be a direct SARM1 activator, e.g., an activator or agonist that binds directly to SARM1 to activate it, such as a molecule that binds to the allosteric pocket of SARM1 (the activation pocket in SARM1's ARM domain). Examples of direct SARM1 activators are the mononucleotides NMN, VMN and 3-APMN which bind to the activation pocket in the N-terminal auto-regulatory ARM domain of SARM1. A SARM1 agent may also be a molecule that binds to the allosteric pocket in the N-terminal auto-regulatory ARM domain of SARM1 in a similar manner to NMN and/or VMN, interacting with some or all of the residues involved in VMN or NMN binding to SARM1. The list of NMN and/or VMN residues interacting with SARM1 can be found in FIG. 16 and in Figley et al. (Neuron. 2021 Apr. 7; 109 (7): 1118-1136.e11), Loreto et al. (Elife. 2021 Dec. 6; 10:e72823), and Shi et al., (Mol Cell. 2022 May 5; 82 (9): 1643-1659.e10).

A SARM1 agent may also be a molecule which does not bind to the allosteric pocket of SARM1 such as Vacor, 3-AP, and analogs or derivatives thereof as well as other molecules disclosed herein. For instance, Vacor is converted into the metabolite VMN (Vacor mononucleotide) and 3-AP is converted into 3-APMN. These metabolites (e.g. NMN, VMN, 3-APMN and others listed below) act as activators that bind to the activation pocket in the N-terminal auto-regulatory ARM domain of SARM1, causing SARM1-dependent neurodegeneration and/or neuronal dysfunction. A SARM1 agent may also be a prodrug which needs to be converted inside the cell to lead to SARM1-dependent neurodegeneration and/or neuronal dysfunction. Vacor, 3-AP, 2-AnP and NMN precursors (e.g. nicotinamide riboside and others listed below) are examples of prodrugs, as they do not activate SARM1 directly, but are converted into direct SARM1 activators, such as VMN, 3-APMN and NMN in the cell.

A SARM1 agent may be a pyridine derivative such as Vacor. It may be a nicotinamide analog such as Vacor. Any pyridine derivatives, analogs of Vacor, 3-AP, nicotinamide, nicotinamide riboside, vacor riboside, vacor riboside analogs, nicotinamide riboside analogs; nicotinic acid analogs; nicotinic acid riboside analogs, nicotinic acid mononucleotide analogs, have the potential to interact with SARM1 or generate a metabolite that acts as a SARM1 activator. The SARM1 agent may also be pyridoxine or 6-aminonicotinamide. In general, SARM1 agents may have a pyridine ring in common. A SARM1 agent may also be an analog of the mononucleotides known to directly activate SARM1, such as NMN, VMN and 3-APMN. In general, examples of analogs of the mononucleotides NMN, VMN, 3-APMN may consist of a sugar (generally ribose), a phosphate group and a nitrogenous base (e.g. a pyridine). These analogs may be SARM1 agents and may activate SARM1 by direct binding or indirectly and cause SARM1-dependent neurodegeneration and/or neuronal dysfunction.

A SARM1 agent may be an analog of prodrugs such as Vacor, 3-AP, 2-AnP and NMN precursors (e.g. analogs of nicotinamide, nicotinamide riboside and other listed below), and generally pyridines and molecules containing pyridine rings. These analogs may activate SARM1 by direct binding or indirectly and cause SARM1-dependent neurodegeneration and/or neuronal dysfunction.

SARM1 is a prodegenerative enzyme with NAD(P)⁺-consuming activity. This activity is regulated by the axonal survival and NAD⁺-synthesising enzyme NMNAT2. NMNAT2 depletion or inhibition leads to an accumulation of NMNAT2 substrate NMN and an increase in NMN/NAD ratio. When NMN levels are high, NMN binds to an allosteric pocket on SARM1 ARM domain (activation pocket) and activates SARM1, leading to increased SARM1-dependent consumption of NAD(P)+ and neurodegeneration. SARM1 agents such as exogenous molecules like vacor and 3-AP may therefore be converted in cells into metabolites, i.e., NMN analogs (VMN and 3-APMN in the present case) which also bind and activate SARM1, causing neurodegeneration. SARM1 agents may therefore cause neurodegeneration and/or neuronal dysfunction in the manner described. A SARM1 agent may be an indirect SARM1 activator which acts via reducing levels and/or enzymatic activity of NMNAT1-2-3 (in particular the axonal isoform and endogenous SARM1 regulator NMNAT2), leading to SARM1 activation and SARM1-dependent neurodegeneration and/or neuronal dysfunction. These agents could be direct inhibitors of NMNAT1-2-3, resulting in loss of enzymatic activity, or molecules that deplete the levels of NMNAT1-2-3. There are various ways in which this could occur, including mitochondrial dysfunction, protein synthesis block, increased protein degradation, impaired axonal transport, NMNAT1-2-3 mutations, molecules that affect NMNAT1-2-3 stability (e.g. effects on turnover, degradation, half-life, stability) and selective inhibition of NMNAT1-2-3 activity (Merlini et al., 2022). A SARM1 agent may encompass any agent that results in SARM1 activation via any one or more of these mechanisms. SARM1 activation may be determined by any means known in the art, as described below.

A SARM1 agent may be an indirect SARM1 activator which activates SARM1 and cause SARM1-dependent neurodegeneration and/or neuronal dysfunction via an alternative mechanism (not direct binding to SARM1), via one or more binding partners or regulate the activity of SARM1 via other molecules. Examples of indirect SARM1 agents include chemotherapy drugs such as vincristine, which impair axonal transport and lead to a reduction in the levels of the endogenous SARM1 regulator NMNAT2. Other examples are mitochondrial toxins such as CCCP, rotenone and others, which also result in a reduction of NMNAT2 levels (Merlini et al. 2022).

Importantly, changes in NMN and NAD levels can trigger SARM1 activation and SARM1-dependent neurodegeneration and/or neuronal dysfunction (Gilley and Coleman, 2010; Figley et al 2021; Coleman & hoke, 2020). Thus, a SARM1 agent may be a molecule that leads to changes in NMN, NAD and NMN/NAD ratio, leading to SARM1 activation and SARM1-dependent neurodegeneration and/or neuronal dysfunction. Specifically, molecules that lead to an increase in NMN levels, a reduction in NAD levels and/or an increase in the NMN/NAD ratio have the potential to activate SARM1. These molecules could act on NMNAT1-2-3 levels and/or activities as described above, but also via other mechanism, such as through activation of NMN synthesising enzymes such as NAMPT (e.g. SBI-797812, PC73 Gardell, S. J., Hopf, M., Khan, A. et al. Boosting NAD+ with a small molecule that activates NAMPT. Nat Commun 10, 3241 (2019) https://doi.org/10.1038/s41467-019-11078-z, and Wang G, Han T, Nijhawan D, et al. P7C3 neuroprotective chemicals function by activating the rate-limiting enzyme in NAD salvage. Cell. 2014; 158 (6): 1324-1334. doi: 10.1016/j.cell.2014.07.040) and/or NRK1,2 activation and/or activation and/or increased expression of NAD+ consuming enzymes such as poly(ADP-ribose) polymerase enzymes (PARPs), Sirtuins and CD38 (Xie et al. 2020). A SARM1 agent may encompass any agent that results in SARM1 activation via any one or more of these mechanisms.

A SARM1 agent may be a molecule that does not lead to SARM1 activation (see definition of SARM1 activation), but still leads to SARM1 dependent neurodegeneration and/or neuronal dysfunction either solely or in combination with any of the other SARM1 agents and/or SARM1-dependent neurodegenerative mechanisms described herein. For instance, SARM1 has base exchange activity, and can convert a molecule into a toxic metabolite even via basal activity. These products could contribute to SARM1-dependent neurodegeneration and/or neuronal dysfunction. For instance, Vacor is used for base exchange to produce VAD. VAD is known to inhibit enzymes in NAD biosynthetic pathway such as NMNAT1-2-3 (Loreto et al 2021 elife). This could reduce compensatory mechanism downstream of SARM1 activation and contribute to the toxicity and SARM1-dependent neurodegeneration and/or neuronal dysfunction caused by vacor and analogs thereof. A list of other molecules that are used by SARM1 for base exchange and could potentially be SARM1 agents can be found in (Angeletti et al 2022, iScience and https://elifesciences.org/articles/67381.pdf), and in table S2 (Shi et al. 2022 Mol Cell).

A SARM1 agent may be any molecule that causes a change in SARM1 enzymatic activity causing SARM1 dependent neurodegeneration and/or neuronal dysfunction. The action could be on any of the enzymatic activities described for SARM1 (NAD(P)-consuming activity, NAD(P) hydrolysis, cyclization and base exchange activity (for more information see Angeletti et al. 2022, iScience) SARM1 dependent neurodegeneration and/or neuronal dysfunction and activation may be determined by any means known in the art, as described below.

A SARM1 agent may be any molecule that causes SARM1 dependent neurodegeneration and/or neuronal dysfunction. SARM-1 dependent neurodegeneration and/or neuronal dysfunction may be determined by any means known in the art as described below.

A SARM1 agent may bind directly or indirectly to a molecule comprising the amino acid sequence of the amino acid sequence identified by UniProtKb number Q6SZW1. The SARM1 agent may bind directly or indirectly to a molecule encoded by a nucleotide sequence encoding a SARM1 protein sequence identified by UniProtKb number Q6SZW1. SARM1 may comprise a sequence that is at least 85%, 90%, 95%, 96%, 97%, 98%, or 99% identical to the sequence identified by UniProtKb number Q6SZW1. SARM1 may be encoded by a nucleic acid sequence that is at least 85%, 90%, 95%, 96%, 97%, 98%, or 99% identical to a nucleic acid sequence encoding a SARM1 protein as identified by UniProtKb number Q6SZW1. In some examples, the SARM1 protein may be a homologue or variant of the protein identified by UniProtKb number Q6SZW1.

As disclosed herein, a SARM1 agent may be a mononucleotide e.g., NMN, CZ-48, VMN and 3-APMN. VMN and 3-APMN are mononucleotides containing a negative charge. The charge of mononucleotides can be adjusted to increase permeability and/or solubility using any means known in the art. Accordingly, analogs and derivatives of SARM1 agents that increase permeability and/or solubility of the SARM1 agent may be useful in the invention. For instance, CZ-48 is a modified version of NMN, where the ribose is replaced with 2-deoxy-2-fluoro-D-arabinose, and one of the oxygens of the phosphate is substituted with a sulfur to increase cellular permeability (https://www.ncbi.nlm.nih.gov/pmc/articles/PMC6531917). These (or similar) modifications could be applied to any SARM1 agent, such as activators of SRAM1 to increase permeability, including VMN, 3-APMN and analogs thereof. Permeability could also be enhanced by common known methods such as by the use of permeation enhancers, ion-pairing, encapsulation (such as nanoencapsulation) or nanosizing.

Accordingly, the SARM1 agent to be used in any methods disclosed herein, may be selected from vacor; vacor mononucleotide (VMN); 3-acetylpyridine (3-AP); 3-acetylpyridine mononucleotide (3-APMN); 2-aminopyridine (2-AnP); 2-AnP mononucleotide (2-AnPMN); nicotinamide mononucleotide (NMN), sulfo-ara-F-NMN (CZ-48); sulfo-ara-F-VMN; sulfo-ara-F-3-APMN; sulfo-ara-F-2-AnPMN, S-NMN, ara-F-NMN (CZ-17); pyridines and molecules containing pyridine rings; vacor riboside (VR), vacor riboside analogs; nicotinamide (NAM) analogs; nicotinamide riboside (NR), nicotinamide riboside analogs; nicotinic acid (NA) analogs; nicotinic acid riboside (NaR) analogs, nicotinic acid mononucleotide (NaMN) analogs, molecules that increase intracellular levels of NMN, decrease NAD levels and/or increase NMN/NAD ratio resulting in SARM1 activation; NMNAT1-2-3 inhibitors or molecules that reduce NMNAT1-2-3 levels; and/or metabolites, analogs and derivatives thereof.

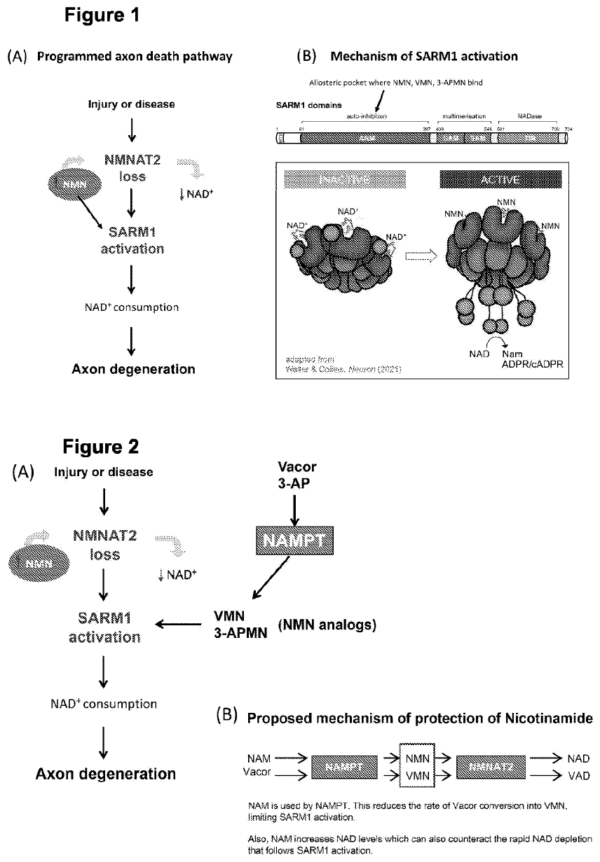

Vacor analogs may work in a similar manner to each other. That is, Vacor analogs may be converted into a metabolite by NAMPT, NRK (nicotinamide riboside kinase) or NaPRT, or any other similar enzyme. The resulting metabolite/mononucleotide may then activate SARM1 through binding to the ARM domain, causing neurodegeneration and/or neuronal dysfunction. For example, in the case of Vacor riboside, the Vacor riboside may converted to VMN in cells by NRK. Other SARM1 agents may interact with SARM1 in a different manner, for example, by binding to another region of SARM1. As an example, Vacor may also be used directly by SARM1 for base exchange (where no conversion into VMN is needed). This reaction is mediated by interaction with another SARM1 domain (the TIR domain) (FIG. 1) (Angeletti et al.2022 iScience). The use of Vacor by SARM1 for base exchange activity may not be sufficient to lead to neurodegeneration and/or neuronal dysfunction alone but in combination with any or all of the above mechanism. Vacor may need to be converted into VMN for toxicity. A SARM1 agent may nevertheless lead to neurodegeneration and/or neuronal dysfunction without the need of conversion into a metabolite, for example through its use by SARM1 for base exchange. Therefore, a SARM1 agent may be a molecule that interacts with SARM1 in any domain of the enzyme, not limited to the activation pocket in the N-terminal ARM domain.

SARM1 possess basal enzymatic activity (see definition above). A SARM1 agent may be a molecule that is converted into a toxic metabolite by SARM1 via base exchange (either activated SARM1 and/or via SARM1 basal activity). The product of this activity can cause SARM1-dependent neurodegeneration and/or neuronal dysfunction, for instance by blocking enzymes in NAD biosynthetic pathway and therefore reducing compensatory mechanism downstream of SARM1 and NAD(P) consumption.

A SARM1 agent may be a molecule that blocks compensatory mechanisms, such as impairs the levels and or activities of enzymes involved in NAD biosynthesis. In this scenario, SARM1 basal activity could result in NAD depletion, leading to SARM1-dependent neurodegeneration and/or neuronal dysfunction.

A SARM1 agent may be a combination of some, all or any of the mentioned approaches to achieve the desired therapeutic outcome. This includes combining more than one SARM1 agent, and also co-administering reversal agents.

For all the applications above, SARM1 activation and/or SARM1 dependent neurodegeneration and/or neuronal dysfunction may be determined by any means known in the art. As described, changes in the levels of NMN, NAD, NMN/NAD ratio, SARM1 products such as Nicotinamide, cADPR, ADPR, cADPRP, ADPRP are indicators of SARM1 activation. Specifically, an increase in NMN, NMN/NAD ratio, nicotinamide, CADPR, ADPR, cADPRP, ADPRP suggests SARM1 activation. A decrease in NAD, NADP and NMNAT1-2-3 levels also suggests SARM1 activation (Figley et al. 2021 Neuron, Angeletti et al. 2021 iScience). The amount of NMN and NAD can be measured using any method known in the art. The ratio of NMN/NAD and an increase in the ratio can in turn be determined from measuring the amounts of NMN and NAD. An increase or decrease in levels of molecules such as NMN, NAD, Nicotinamide, cADPR, ADPR, cADPRP, ADPRP or NMNAT2 can be determined by methods known in the art. Examples of assays to determine SARM1 activation and/or SARM1 dependent neurodegeneration and neuronal dysfunction are metabolomics analysis using NAD glo assay kit, HPLC and LC-MS to measure changes in levels of NMN, NAD, NMN/NAD ratio and changes in levels of products of SARM1 activity, Nicotinamide, cADPR, ADPR, cADPRP, ADPRP (Angeletti et al., 2022 iSciece; Figley et al. 2021 Neuron, Loreto et al. 2021 elife and https://pubmed.ncbi.nlm.nih.gov/32087251/). Other examples are the use of live fluorescent probes e.g. PC6 as described in (https://elifesciences.org/articles/67381.pdf). Other examples are the use of an antibody that binds to SARM1 active conformation (e.g. Nb-C60) as described in (https://www.biorxiv.org/content/10.1101/2022.03.25.485784v2.full). Other examples are the use of cryo-EM and x-ray crystallography to study structural changes that are associated with SARM1 activation (Loreto et al. 2021 elife, Figley et al 2021 Neuron, Shi et al. 2022 Mol Cell). Other examples are enzymatic assays of recombinant SARM1 to study molecules that affect SARM1 activity as described (Angeletti et al. 2022 iScience, Loreto et al 2021 elife; Gilley et al. 2021 elife). Other examples are methods to detect levels and/or enzymatic activities of NMNAT1-2-3, such as western blot and enzymatic assays. An increase or decrease of the molecules mentioned above can be determined by reference to a baseline level or amount before administration of a SARM1 agent. The control sample could be the levels of NMN, NAD, NMN/NAD ratio, NMNAT1-2-3, SARM1 products such as Nicotinamide, cADPR, ADPR, cADPRP, ADPRP NMN, NAD, CADRP, ADPR and other markers of SARM1 activation disclosed herein prior to SARM1 activation/addition of SARM1 agent to a sample. For instance, an increase in levels of the molecules specified above could be at least about 3% greater amount in the sample compared with the control sample, for example at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90% greater than the sample population. A decrease could be at least about 3% lower amount in the sample compared with the control sample, for example at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90% lower than the sample population. For instance, an increase in levels could be at least about 1.1 fold greater, up to 10 fold. For instance, a decrease in levels could be at least about 1.1 fold reduction, up to 10 fold reduction. For instance, an increase in NMN, NMN/NAD ratio, nicotinamide, cADPR, ADPR, cADPRP, ADPRP of about 3% or greater may identify a molecule, metabolite, analog or derivative thereof as a SARM1 agent. A decrease in levels of NAD, NADP and NMNAT1-2-3 of about 3% or greater may identify a molecule, metabolite, analog or derivative thereof as a SARM1 agent. A decrease in the level of NMNAT2 of about 3% or greater may identify a molecule, metabolite, analog or derivative thereof as a SARM1 agent. An increase in the ratio of NMN/NAD of 3% or greater may identify a molecule, metabolite, analog or derivative thereof as a SARM1 agent. In addition, SARM1-dependent neurodegeneration and/or neuronal dysfunction can be determined using any means known in the art. Examples are studies of neuronal and/or neurite morphology using microscopy (Loreto et al. 2021 eLife), measuring levels of neurofilament light chain (NfL) in in vitro assay, (e.g. culture supernatants) serum and Cerebrospinal fluid (CSF) (https://www.sciencedirect.com/science/article/pii/S2211124720315771?via%3Dihub), metabolomics assays as described above, electrophysiology-nerve conduction studies/electromyography, mitochondrial dynamics and function, axonal transport, metabolomic changes, serum/CSF biomarkers, such as neurofilament. Motor/sensory neurological examination including MRC scales and sum score, cognitive testing, such as Addenbrookes cognitive exam III, methods to measure peak volitional muscle contraction force, such as dynamometry and muscle fatigue, such as neurological examination and neurophysiology, timed walking tests, objective sensory examinations such as Von frey hair, thermal thresholds, proprioceptive tests, such as the 9 hole peg test. Intraepidermal nerve fibre analysis.

The SARM1 agent may be any one of more of the following compounds:

### Vacor

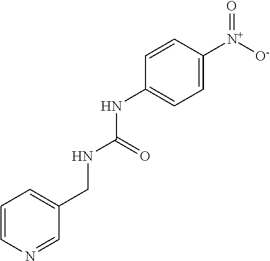 [SMILES: O=C(NCc1cccnc1)Nc1ccc([N+](=O)[O-])cc1]

Vacor may be converted into VMN for toxicity in neurons. Vacor may interact with SARM1 active site (TIR domain) for base exchange reaction which results in a modest increase in SARM1 activity. (Loreto et al., 2021; Angeletti et al., 2022).

### VMN (Vacor Mononucleotide)

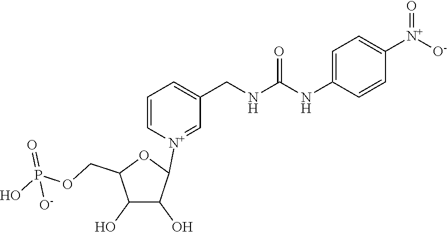 [SMILES: O=C(NCc1ccc[n+](C2OC(COP(=O)([O-])O)C(O)C2O)c1)Nc1ccc([N+](=O)[O-])cc1]

VMN is a Vacor metabolite, and binds SARM1 in the allosteric site (ARM domain) and potently activates SARM1. It is most likely not cell permeable (Loreto et al., 2021, ELife December 6) but may have cell permeability increased by common known methods such as by the use of permeation enhancers, ion-pairing, encapsulation (such as nanoencapsulation) or nanosizing. For instance, CZ-48 is a modified version of NMN, where the ribose is replaced with 2-deoxy-2-fluoro-D-arabinose, and one of the oxygens of the phosphate is substituted with a sulfur to increase cellular permeability. The same modfications could be used for VMN and other mononucleotides listed herein.

3-acetylpyridine (3-AP)

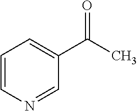

3-AP needs to be converted into 3-APMN for toxicity in neurons. 3-AP may interact with the SARM1 active site (TIR domain) for base exchange reaction, which results in an increase in SARM1 activity. (Angeletti et al., 2022, iScience January 25; 25(2) 103812; Wu et al., 2021, Cell Rep, October 19:37(3); 109872).

### 3-AP Mononucleotide (3-APMN)

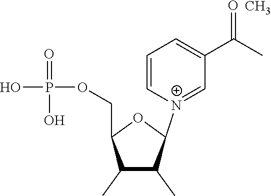

3-APMN is a 3-AP metabolite, predicted to bind SARM1 in the allosteric site (ARM domain) and activate SARM1. Most likely not cell permeable (Wu et al., 2021) but may have cell permeability increased by common known methods such as by the use of permeation enhancers, ion-pairing, encapsulation (such as nanoencapsulation) or nanosizing. For instance, CZ-48 is a modified version of NMN, where the ribose is replaced with 2-deoxy-2-fluoro-D-arabinose, and one of the oxygens of the phosphate is substituted with a sulfur to increase cellular permeability. The same modfications could be used for 3-APMN and other mononucleotides listed herein.

2-aminopyridine (2-AnP). 2-AnP may bind to SARM1's allosteric site and trigger SARM1-dependent neurodegeneration such as axon degeneration.

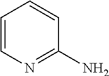

### Nicotinamide Mononucleotide (NMN)

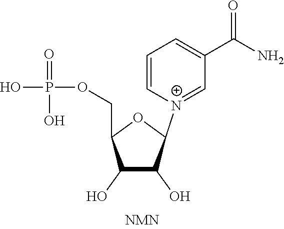

NMN is an endogenous SARM1 agent/activator. It binds SARM1 directly in the allosteric site (ARM domain) and potently activates it. Precursors of NMN are nicotinamide and nicotinamide riboside (both part of the vitamin B3 family) (Figley et al., 2021, Neuron, April 7:109(7): 1118-1136). Most likely not cell permeable (Wu et al., 2021) but may have cell permeability increased by common known methods such as by the use of permeation enhancers, ion-pairing, encapsulation (such as nanoencapsulation) or nanosizing. For instance, CZ-48 is a modified version of NMN, where the ribose is replaced with 2-deoxy-2-fluoro-D-arabinose, and one of the oxygens of the phosphate is substituted with a sulfur to increase cellular permeability.

CZ-48 (sulfo-ara-F-NMN)

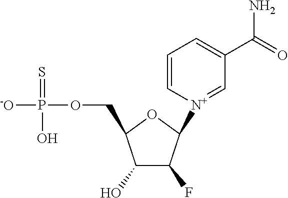

CZ-48 is an NMN analog. CZ-48 can activate SARM1 both in in vitro assays and in cells. It is modified (F and S) to improve cell permeability. (Zhao et al., 2019, iScience May 31; 15:452-466).

CZ-17 (ara-F-NMN)

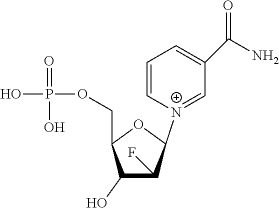

C-17 is an NMN analog. C-17 can activate SARM1 in in vitro assays (Zhao et al., 2019).

### S-NMN

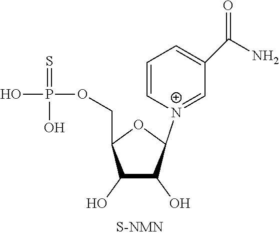

S-NMN is an NMN analog. It can activate SARM1 in in vitro assays (Zhao et al., 2019).

The SARM1 agent may include those having chemical modifications of the compounds disclosed herein. The SARM1 agent may also be a derivative, prodrug or analog of any of the compounds disclosed herein. For example, modifying the compounds with a F and/or S group may improve cell permeability as exemplified by CZ-48. Such chemical modifications can be applied to other SARM1 agents such as VMN and 3-APMN.

### A SARM1 Agent for Use as a Medicament

The SARM1 agent may be associated with a first medical use. By way of example, SARM1 agents that lead to activation of SARM1 such as Vacor and related compounds have been used in non-medical uses such as in pest control as a rodenticide. Such SARM1 agents have been identified by the inventors as useful in treating and/or preventing diseases/conditions as disclosed herein for the first time. Accordingly, disclosed herein is a SARM1 agent for use as a medicament.

A SARM1 Agent for Use in a Method of Treating and/or Preventing Disease

A SARM1 agent disclosed herein may be for use in a method of treating and/or preventing disease. The disease may be neurological, ophthalmological, dermatological, gastroenterological, colorectal, urological, gynaecological, rheumatological orthopaedic, dental, and/or ear nose and throat disease.

In some examples, the disease is a SARM1 dependent neurological disease. SARM1 dependent neurological disease refers to any disease or condition that is regulated, caused by or has symptoms associated with dysregulation of SARM1. For example, the disease or condition and any symptoms thereof may be caused by or associated with a decrease in the activity of SARM1. A decrease in activity refers to a decrease in enzymatic activity of a SARM1 protein in a subject, and/or a reduction in expression of SARM1 mRNA. The decrease may be for example at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90% or more in comparison to SARM1 activity in a healthy subject.

The disease or condition to be treated or prevented may also be defined as autonomic dysfunction, neuropathic pain, dystonia, spasticity, dyskinesia, sphincter dysfunction, genitourinary dysfunction and/or a skin condition. The disease or condition may be prevented or treated in whole or in part. That is, any of these diseases or conditions may be wholly absent following treatment or prevention if prevented or treated “in whole”. Alternatively, any of these diseases or conditions may be partly present following treatment or prevention if prevented or treated “in part” if these diseases or conditions may be partly prevented or treated. By way of example, neuropathic pain may be prevented or treated in whole if neuropathic pain is completely absent following treatment or prevention with a SARM1 agent. As a further example, neuropathic pain may be prevented or treated in part if there is reduced neuropathic pain following treatment or prevention with a SARM1 agent.

As used herein, the terms “treat”, “treating” and “treatment” are taken to include an intervention performed with the intention of preventing the development or altering the pathology of a condition, disorder or symptom. Accordingly, “treatment” refers to both therapeutic treatment and prophylactic or preventative measures, wherein the object is to prevent or slow down (lessen) the targeted condition, disorder or symptom. In other words, terms “treatment,” “treat,” and “treating” refer to reversing, alleviating, delaying the onset of, or inhibiting the progress of a condition, symptom or disease.

In some examples, treatment may be administered after one or more signs or symptoms of the disease have developed or have been observed. In other examples, treatment may be administered in the absence of signs or symptoms of the disease. For example, treatment may be administered to a susceptible subject prior to the onset of symptoms (e.g., in light of a history of symptoms). Treatment may also be continued after symptoms have resolved, for example, to delay and/or prevent recurrence. Treatment may bring about prolonged survival as compared to expected survival if not receiving treatment. Alternatively, or additionally, treatment may provide a patient with an improved standard of life as compared to that which would be expected if not receiving treatment.

“autonomic dysfunction”, also known as “dysautonomia” “autonomic neuropathy or ganglionopathy, “primary or “secondary autonomic failure” refers to a condition in which the autonomic nervous system (ANS) does not work properly. This may affect the functioning of the heart, bladder, intestines, sweat glands, pupils, smooth muscles, and/or blood vessels. Autonomic dysfunction has many causes, not all of which may be classified as neuropathic. The primary symptoms of autonomic dysfunction, which can vary between individuals, include: orthostatic hypotension; syncope and pre-syncope; hypothermia; hyperthermia; dry mouth; siallorhoea; rapid/slow/irregular heart rate; pupillary dysfunction; labile blood pressure; difficulty swallowing; bowel incontinence; blurry vision; urinary incontinence, retention and bladder voiding dysfunction; gastroparesis; constipation; postpradial symptoms; anhydrosis; hyperhidrosis and sexual disfunction, such as erectile/clitoral dysfunction and loss of libido, fatigue, exercise intolerance, anxiety, headache, brain fog, scapular, back and neck pain (coat hanger pain). Autonomic dysfunction may be due to inherited, genetic or degenerative neurological diseases (primary dysautonomia) or it may occur due to injury/disease of the autonomic nervous system from an acquired disorder (secondary dysautonomia), such as infections, such as COVID, and long-COVID, postural orthostatic tachycarida syndrome (POTS), autoimmune conditions, haematological conditions, amyloidosis, endocrine conditions such as diabetes and thyroid dysfunction, neuropathies secondary to paraneoplastic, inflammatory, metabolic and vasculitic causes, small fibre neuropathy, connective tissue diseases, such as Ehlers Danlos spectrum disorders, menopause, or medications, the cause may also be unknown (idiopathic)

Autonomic dysfunction may include any one or more of excessive sweating, and/or excessive saliva production also known as sialorrhea. Autonomic dysfunction may be primary or secondary to a neurodegenerative diseases such as amyotrophic lateral sclerosis, Parkinson's disease, parkinsonism, multiple system atrophy, cerebral palsy and drugs.

Known treatments for autonomic dysfunction includes glycopyrronium, Botulinum toxin A/B injection of submandibular/parotid glands for sialorrhea or topical NH3CL antiperspirants/iontophoresis or Botulinum toxin A/B injection of axillary sweat glands and sweat glands elsewhere for hyperhidrosis (excess sweating). The SARM1 agents described herein may be used in addition to or in conjunction with any known treatments for autonomic dysfunction as described above.

Thus in some examples, there is provided a SARM1 agent as described herein for use in treating or preventing or for use in methods of treating or preventing autonomic dysfunction.

Neuropathic pain refers to pain caused by nerve damage or abnormal nerve function, and may also include spontaneous pain, which occurs even when there is no nociception stimulation, allodynia, which is caused by stimuli that normally do not cause pain, hyperalgesia, which is worsened by noxious stimulus, and other abnormal responses, such as paraesthesia or dysesthesia. When the ‘neuropathic pain’ is divided into a central type and a peripheral type, central neuropathic pain may be caused by damage to the central nervous system, which stimulates the spinal cord, brain stem, thalamus, and cortex, such as brain tumour, cerebral haemorrhage, syringomyelia, and acquired immune deficiency syndrome. Peripheral neuropathic pain may refer to pain caused by abnormalities of the peripheral nervous system, such as postherpetic neuralgia, diabetic neuropathy, and complex regional pain syndrome type II (Duck Mi Yoon, 2011).

Neuropathic pain includes peripheral neuropathic pain; chronic back pain; cancer related pain; myofascial pain secondary to bruxism or temporomandibular joint dysfunction; fibromyalgia; complex regional pain disorder; chronic pelvic pain; dyspareunia; vulvodynia; painful bladder syndrome (interstitial cystitis) or the neuropathic pain caused by diabetic neuropathy, neuralgia post peripheral nerve injuries and surgical repair of nerve injuries, infective and post-infective neuralgias, such as post-herpetic neuralgia and Long-COVID, HIV neuropathy and Lymes disease; chemotherapy, toxic and drug induced neuropathies; metabolic neuropathies such as secondary to kidney or liver dysfunction/failure; small fibre neuropathy; endocrine conditions such as thyroid, parathyroid and pituitary dysfunction; genetic neuropathies, such as Charot Marie-Tooth disease, hereditary sensory neuropathies (HSN) and hereditary sensory and autonomic neuropathy (HSAN); paraproteinaemic, paraneoplastic and neoplastic neuropathies; vasculitis; auto-immune, inflammatory and demyelinating neuropathies, such as Guillian-Barre syndrome and Chronic inflammatory demyelinating polyradiculoneuropathy; and idiopathic neuropathies. In some examples, the neuropathic pain is peripheral neuropathic pain.

Cancer pain refers to cancer-induced pain. According to a three-step analgesic ladder created as a guideline for cancer pain management and advocated by the World Health Organization (WHO), it is advised as follows: Step 1: Non-opioid analgesic for mild pain, Step 2: Opioid analgesic for moderate pain, and Step 3: strong opioid analgesic for strong pain. The pain degree is varied, and for example, when the pain is severe, it is regarded as an emergency to be promptly treated, and may be associated with pain that is so severe as to cause general weakness, mental illness, and the like. Thus, such pain may refer to pain requiring active pain relief treatment classified differently from other pain causes.

Known treatments for neuropathic pain such as peripheral neuropathic pain include gabapentin, pregabalin, serotonin noradrenaline reuptake Inhibitors (such as Duoxetine or Venlafaxine), tricyclic antidepressants (which may be first line therapies), capsaicin 8% patches, lidocaine patches, tramadol (which may be second line therapies), or botulinum toxin A (which may be a third line treatment) (Finnerup et al. 2015 Lancet Neurol. 2015 February; 14(2): 162-73, NICE guidelines on neuropathic pain cg173-2013; Attal et al. 2010. Eur J Neurol. 2010 September; 17(9):1113-e88). The SARM1 agents described herein may be used in addition to or in conjunction with any known treatments for neuropathic pain as described above.

In some examples, there is provided a SARM1 agent as described herein for use in treating or preventing or for use in methods of treating or preventing neuropathic pain. In some examples, the SARM1 agents and compositions described herein may be administered by a transdermal patch for use in treating neuropathic pain.

Dystonia refers to sustained muscle contractions that cause repetitive movements or twisting and other abnormal postures. In some embodiments, a dystonia can occur in a limb, e.g., a hand or foot.

Dystonia includes primary dystonia, such as focal, segmental, genetic and idiopathic types such as blepharospasm, strabismus, oromandibular dystonia, laryngeal dystonia, writer's cramp and cervical dystonia; dystonia plus conditions (such as dopa-responsive dystonia (DRD) or Segawa syndrome, rapid-onset dystonia-parkinsonism (RDP) and myoclonus-dystonia), and dystonia occurring with neurodegenerative diseases such as Parkinson's disease, multiple system atrophy, Huntington's disease, Wilson's disease and neuroferritinopathy; secondary dystonia occurring due to central nervous system (CNS) trauma, congenital malformation, genetic and chromosomal disorders, infection, neoplasm, ischaemic or haemorrhagic stroke, inflammation, demyelination, drugs, toxins, metabolic disturbance such as secondary to kidney or liver dysfunction/failure, paraneoplastic, post-infections autoimmune and endocrine conditions such as thyroid, parathyroid and pituitary dysfunction.

Known treatments for dystonia include botulinum toxin A (for cranial/cervical dystonia); botulinum toxin B (for cervical dystonia), sometimes combined with adjunctive oral medications, such as trihexyphenidyl, dopamine replacement or agonists drugs, benzodiazepine drugs, such as diazepamas well as surgical intervention such as deep brain stimulation. (DBS), e.g. Palladial DBS for cervical dystonia (Phukan et al. 2011. Lancet Neurol. December; 10 (12): 1074-85, Marion et al. 2016. Pract Neurol 2016; 16:288-295). The SARM1 agents described herein may be used in addition to or in conjunction with any known treatments for dystonia as described above.

In some examples, there is provided a SARM1 agent as described herein for use in treating or preventing or for use in methods of treating or preventing dystonia.

Spasticity refers to a condition in which certain muscles are continuously or abnormally contracted. This contraction causes stiffness or tightness of the muscles and can interfere with normal movement of face, limbs, trunk, and/or sphincters, leading to deficits in, for example, speech, gait, and/or bladder and bowel function. Spasticity is a condition that occurs in a number of disorders of the CNS that affect brain and/or spinal cord function, including, for example traumatic injury to brain or spinal cord, multiple sclerosis, cerebral palsy, stroke, or other conditions. Despite the underlying condition, spasticity develops when the properties of motor neurons change in response to the condition, and over-produce electrical impulses, leading to excessive muscle contraction. The damage causes a change in the balance of signals between the nervous system and the muscles, leading to increased excitability in muscles. Spasticity is found in conditions where the brain and/or spinal cord are damaged or fail to develop normally; these include cerebral palsy, multiple sclerosis, spinal cord injury, and acquired brain injury including stroke.

Spasticity may include one or more of upper limb spasticity, lower limb spasticity or bladder spasticity/neurogenic bladder; spastic disorders of the oesophagus such as oesophageal spasm and nutcracker oesophagus; or spasticity caused by ischaemic or haemorrhagic stroke; demyelinating conditions, such as multiple sclerosis; traumatic spinal cord or brain injury; congenital malformation such as cerebral palsy; chromosomal disorders; genetic conditions, such as hereditary spastic paraplegia; CNS infections; neoplasm; drugs and toxins; metabolic disturbance such as secondary to kidney or liver dysfunction/failure; paraneoplastic disorders; post-infections conditions; autoimmune conditions; endocrine conditions such as thyroid, parathyroid and pituitary dysfunction.

Known treatments for spasticity include oral and intrathecal Baclofen, benzodiazepines, Tizanidine, cannabinoids (such as Nabixomols), dantrolene or botulinum toxin A/B, intraneural, intramuscular and intrathecal phenol injection as well as surgical intervention such as rhizotomy (Kheder et al., Pract Neurol. 2012 October; 12(5): 289-98, Chang et al. 2013. Crit Rev Phys Rehabil Med. 2013; 25(1-2): 11-22). The SARM1 agents described herein may be used in addition to or in conjunction with any known treatments for spasticity as described above.

In some examples, there is provided a SARM1 agent as described herein for use in treating or preventing or for use in methods of treating or preventing spasticity.

Dyskinesia refers to anomalous or uncontrollable movements, including, but not limited to, chorea, tremor, ballismus, dystonia, athetosis, myoclonus and tics.

Dyskinesia may include one or more of hemifacial spasm and facial nerve synkinesis; palatal myoclonus/tremor; tic disorders such as motor and phonic tics in Tourette's syndrome; tremor such as essential, dystonic, parkinsonian and rubral tremor; myokymia such as idiopathic eyelid, facial, limb myokymia or secondary to tumour, trauma, infection, autoimmune conditions including autoimmune encephalitis, demyelination such as multiple sclerosis and neuropathies such as Guillian-Barre syndrome; neuromyotonia caused by autoimmune, paraneoplastic or hereditary conditions (Fuller. 2010. Practical Neurology 10:114-123).

Known treatments for dyskinesias include use of beta-blocking drugs, gabapentin, opiod drugs, benzodiazepine and barbiturate drugs, anticholinergic drugs such as procyclidine and trihexyphenidyl, dopamine antagonist drugs, tetrabenazine, botulinum toxin A/B and deep brain stimulation (Fuller. 2010. Practical Neurology 10:114-123.).

Neuromyotonia is rare condition of spontaneous, continuous muscle activity of peripheral nerve origin. It is characterized clinically by muscle twitching at rest (visible myokymia), cramps that can be triggered by voluntary or induced muscle contraction, and impaired muscle relaxation (pseudomyotonia) (Maddison Practical Neurology, 2002, 2, 225-229).

Known treatments for neuromyotonia include anticonvulsant drugs and immunosuppressive therapy such as steroids, immunoglobulins, azathioprine and plasma exchange (Maddison Practical Neurology, 2002, 2, 225-229).

In some examples, there is provided a SARM1 agent as described herein for use in treating or preventing or for use in methods of treating or preventing dyskinesia.

Sphincter dysfunction may be caused by dysfunction of bodily sphincters including the oesophageal sphincter such as achalasia caused by genetic/hereditary, infections such as Chagas disease and autoimmune conditions in addition to idiopathic achalasia; pyloric sphincter such as gastroparesis caused by conditions such as diabetes; sphincter of Oddi dysfunction leading to gastrointestinal dysmotility; pancreatic sphincter such as recurrent pancreatitis; urethral sphincter such as detrusor sphincter dyssynergia either primary or secondary to brain or spinal cord injury, multiple sclerosis, haemorrhagic or ischaemic stroke, CNS tumour, Fowler syndrome; anal sphincter dysfunction such as anismus; anal fissure, either primary or secondary to inflammatory bowel disease, HIV infection, anal abscess and perianal tumour/cancer; and Hirshsprungs disease (Bach and Simman, Plast Reconstr Surg Glob Open. 2022 Apr. 6; 10(4):e4228, Apostolidis A., Eur Urol. 2009 January; 55(1): 100-19, Lacy et al. Gastroenterol Hepatol (N Y). 2008 April; 4(4): 283-295).

Known treatments for sphincter dysfunction include botulinum toxin A/B injection and surgical options (Bach and Simman, Plast Reconstr Surg Glob Open. 2022 April 6; 10(4):e4228, Apostolidis A., Eur Urol. 2009 January; 55(1): 100-19, Lacy et al. Gastroenterol Hepatol (N Y). 2008 April; 4(4): 283-295).

In some examples, there is provided a SARM1 agent as described herein for use in treating or preventing or for use in methods of treating or preventing sphincter dysfunction.

Genitourinary dysfunction refers to conditions that effect the urinary and genital organs, for example, vaginismus either primary or secondary to childbirth, infection, trauma, iatrogenic or psychological conditions; voiding dysfunction such as after pelvic surgery; pelvic floor muscle dysfunction (Moga et al., 2018, Toxins 2018, 10, 169).

Known treatments for genitourinary dysfunction include lubricants, anxiolytics and botulinum toxin A/B injection (Moga et al., 2018, Toxins 2018, 10, 169).

In some examples, there is provided a SARM1 agent as described herein for use in treating or preventing or for use in methods of treating or preventing genitourinary dysfunction.

Skin condition may include one or more of an ulcer, either primary or secondary to diabetes, vascular disease including disorders or arteries and veins, autoimmune conditions, infections, neuropathy and trauma; acne; hypertrophic scars, such as keloid scars; rosacea and facial flushing; pruritus; Hailey-Hailey disease; hidradenitis suppurativa; alopecia; notalgia paraesthetica; psoriasis; eczema; raynauds phenomenon/disease either primary or secondary to autoimmune disease such as systemic lupus erythematosus and limited and systemic sclerosis (see Kim et al., 2017.doi: 10.3390/toxins9120403; Bach and Simman, Plast Reconstr Surg Glob Open. 2022 Apr. 6; 10 (4):e4228; Campanati et al., 2017 doi: 10.1159/000452341; and Winayanuwattikun and Vachiramon 2022. doi: 10.3390/toxins14060406).

In some examples, there is provided a SARM1 agent as described herein for use in treating or preventing or for use in methods of treating or preventing skin conditions.

Orthopaedic disease or conditions may refer to sports injuries; posttraumatic elbow stiffness; club foot; and/or piriformis syndrome (REFBach and Simman, Plast Reconstr Surg Glob Open. 2022 Apr. 6; 10 (4):e4228).

In some examples, there is provided a SARM1 agent as described herein for use in treating or preventing or for use in methods of treating or preventing orthopedic diseases or conditions.

Dental disease or conditions may refer to odontalgia, root canal procedures, and/or dental surgery (Pergolozzi et al., Expert Opin Pharmacother. 2020 April; 21(5): 591-601. In some examples, there is provided a SARM1 agent as described herein for use in treating or preventing or for use in methods of treating or preventing dental diseases or conditions. In some examples, there is provided a SARM1 agent for use in a method of surgery such as dental surgery. In some examples, there is provided the use of a SARM1 agent in surgery such as dental surgery.

Therapeutic treatment may result in a decrease in severity of disease symptoms, or an increase in frequency or duration of symptom-free periods. An amount adequate to accomplish this is defined as “therapeutically effective amount”. In prophylactic applications, SARM1 agents and any products containing SARM1 agents are administered to a subject not yet exhibiting symptoms of a disorder or condition, in an amount sufficient to prevent or delay the development of symptoms. Such an amount is defined as a “prophylactically effective amount”. The subject may have been identified as being at risk of developing the disease or condition by any suitable means. Thus the invention also provides a SARM1 agent disclosed herein for use in the treatment of the human or animal body.

In some examples, the SARM1 agents described herein and compositions described herein may be for use in methods of treating one or more of strabismus, blepharospasm, cervical dystonia, upper limb spasticity, lower limb spasticity, overactive bladder, neurogenic bladder, hyperhidrosis, sialorrhea, neuropathic pain (e.g. peripheral neuropathic pain).

Also provided herein is a method of prevention or treatment of disease or condition as disclosed herein, in a subject, which method comprises administering a SARM1 agent to the subject in a prophylactically or therapeutically effective amount. The SARM1 agent may be co-administered with another agent. The method of administering a SARM1 agent may be carried out by any suitable administration route or as disclosed herein.

A suitable dosage of a SARM1 agent of the invention may be determined by a skilled medical practitioner. Actual dosage levels of a SARM1 agent may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular antibody employed, the route of administration, the time of administration, the rate of excretion of the antibody, the duration of the treatment, other drugs, compounds and/or materials used in combination with the particular compositions employed, the age, sex, weight, condition, general health and prior medical history of the patient being treated, and like factors well known in the medical arts.

A suitable dose of a SARM1 agent may be, for example, in the range of from about 0.1 μg/kg to about 100 mg/kg body weight of the patient to be treated. For example, a suitable dosage may be from about 1 μg/kg to about 10 mg/kg body weight per day or from about 10 μg/kg to about 5 mg/kg body weight per day. The amount of SARM1 agent that is administered may be of any appropriate amount such as between 0.01 mg/kg body weight and 2 mg/kg body weight, between 0.04 and 2 mg/kg body weight, between 0.12 mg/kg body weight and 2 mg/kg body weight, preferably between 0.24 mg/kg and 2 mg/kg body weight and most preferably between 1 mg/kg and 2 mg/kg body weight. The SARM1 agent may be administered on multiple occasions to the same subject.

Dosage regimens may be adjusted to provide the optimum desired response (e.g., a therapeutic response). For example, a single bolus may be administered, or the method may comprise several divided doses administered over time or the dose may be proportionally reduced or increased as indicated by the exigencies of the therapeutic situation, provided the required interval. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Any of the SARM1 agents described herein can be formulated as a vaccine, to be administered for prophylaxis or as a treatment. Any such vaccine formulation may be administered directly to the animal for prophylaxis or as treatment. Any such vaccine formulation may be administered to the muscle of a subject. For example, the vaccine formulation may be administered, e.g. injected, into facial muscles. Any such vaccine formulation may be in the form of a composition comprising the SARM1 agent of the invention.

The invention also provides a SARM1 agent for use in the manufacture of a medicament for the treatment or prevention of a disease or condition as disclosed herein. The invention also provides for a method of treating and/or preventing a disease or condition as disclosed herein by administration of a SARM1 agent in a subject in need thereof. Accordingly, any reference to or embodiments relating to the use of SARM1 agent for use in treating and/or preventing a disease or condition as disclosed herein can be applied to a SARM1 agent for use in the manufacture of a medicament for the treatment or prevention of a disease or condition and methods of treating and/or preventing a disease or condition.

The SARM1 agents described herein may be part of a pharmaceutical composition. As used herein, “pharmaceutical composition” refers to a composition comprising one or more compounds that is formulated for administration to a subject. Pharmaceutical compositions typically comprise one or more active ingredients (e.g. in this case a SARM1 agent) and one or more pharmaceutically acceptable materials. The pharmaceutical compositions described herein may therefore comprise SARM1 agent and one or more other components. For example, the pharmaceutical composition may comprise SARM1 agent and a pharmaceutically acceptable excipient, diluent and/or carrier. Pharmaceutical compositions may routinely contain pharmaceutically acceptable concentrations of salt, buffering agents, preservatives, compatible carriers, supplementary immune potentiating agents such as adjuvants and cytokines and optionally other therapeutic agents or compounds.

As used herein, “pharmaceutically acceptable” refers to a material that is not biologically or otherwise undesirable, i.e., the material may be administered to an individual along with the selected compound (e.g. SARM1 agent) without causing any undesirable biological effects or interacting in a deleterious manner with any of the other components of the pharmaceutical composition in which it is contained.

In some examples, the pharmaceutical composition comprises a pharmaceutically acceptable diluent. Diluents are diluting agents. Pharmaceutically acceptable diluents are well known in the art. A suitable diluent is therefore easily identifiable by one of ordinary skill in the art.

In some examples, the pharmaceutical composition comprises a pharmaceutically acceptable excipient. Excipients are natural or synthetic substances formulated alongside an active ingredient included for the purpose of bulking-up the formulation or to confer a therapeutic enhancement on the active ingredient in the final dosage form, such as facilitating drug absorption or solubility. Excipients can also be useful in the manufacturing process, to aid in the handling of the active substance concerned such as by facilitating powder flowability or non-stick properties, in addition to aiding in vitro stability such as prevention of denaturation over the expected shelf life. Pharmaceutically acceptable excipients are well known in the art. A suitable excipient is therefore easily identifiable by one of ordinary skill in the art. By way of example, suitable pharmaceutically acceptable excipients include water, saline, aqueous dextrose, glycerol, ethanol, and the like.

In some examples, the pharmaceutical composition may comprise a SARM1 agent and a pharmaceutically acceptable carrier. Carriers are non-toxic to recipients at the dosages and concentrations employed and are compatible with other ingredients of the formulation. The term “carrier” denotes an organic or inorganic ingredient, natural or synthetic, with which the active ingredient is combined to facilitate the application. Pharmaceutically acceptable carriers are well known in the art. A suitable carrier is therefore easily identifiable by one of ordinary skill in the art.

### Non-Therapeutic Methods

A non-therapeutic method disclosed herein may be for (i) removing and/or preventing skin changes, (ii) contouring, and/or (iii) improving oily skin, the method comprising administering a SARM1 agent.

Skin changes may include one or more of skin wrinkles, frown/glabellar lines, forehead lines, lateral canthal lines/crow's feet, horizontal nasal bridge lines, neck bands, platysmal neck lines, smoker's lines, bunny lines, top lip lines, ptosis of the lateral commissure, marionette lines, gummy smile, chin dimples, peau d'orange chin, pitted skin, eye brow sagging, eyelid sagging, lip sagging, square jaw, and a downturned nasal tip.

Administration of one or more SARM1 agents may thus prevent any one of more skin changes as described herein. Removal of skin changes may therefore be the removal of any one of more of these skin changes for a period of time. Prevention of skin changes may therefore be the prevention of any one or more of these changes for a period of time. That is, the effect of the method may be temporary as disclosed herein.

Contouring may comprise (i) shaping lip, cheek, jaw, and/or temple; and/or (ii) eyebrow contour. One or more SARM1 agents may be used to contour/shape any one or more of these facial features. Contouring/shaping may also be performed on other bodily features such as the gums, neck, chest, abdomen, back, legs, calves, arms, and shoulders. Contouring may be a temporary effect (i.e., lasting a finite period of time), as disclosed herein.

Improvement of oily skin may be the reduction of oil from the skin following application of the non-therapeutic method. This effect may be temporary (i.e., lasting a finite period of time) as disclosed herein.

As disclosed herein, the effect of the non-therapeutic method may be temporary. Temporary may mean that the effect (e.g., removal/prevention of skin wrinkles) lasts up to 30 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 48 hours, 72 hours, 96 hours, 1 week, 2 weeks, 3 weeks, 4 weeks, 5 weeks, 6 weeks, 7 weeks 8 weeks, 3 months, 4 months, 5 months, 6 months, 7 months, 8 months, 9 months, 10 months, 11 months, 12 months, 2 years, 3 years, 4 years, or 5 years following application of the method.

The non-therapeutic method disclosed herein may be for cosmetic purposes only. It may not encompass a medical use.

### Neurodegeneration

The SARM1 agents described herein may lead to SARM1-dependent neurodegeneration and/or neuronal dysfunction and/or lead to SARM1 activation. SARM1 is comprised of an autoinhibitory N-terminal armadillo repeat (ARM) domain, tandem sterile-alpha motif (SAM) domains that mediate octamerization, and a C-terminal Toll/interleukin-1 receptor (TIR) domain which possesses enzymatic activity. In healthy neurons in vivo, SARM1 is maintained in an autoinhibited state via multiple intra- and intermolecular interactions, including binding of the N-terminal ARM domain to the C-terminal TIR-domain. SARM1 autoinhibition is regulated by an allosteric binding site within the autoinhibitory ARM domain that can bind either NAD+ or its precursor, nicotinamide mononucleotide (NMN). Axon injury leads to loss of the NAD+ biosynthetic enzyme nicotinamide mononucleotide adenylyltransferase 2 (NMNAT2), resulting in an increased NMN/NAD+ ratio that favours NMN binding to the allosteric site. The switch from NAD+ to NMN binding induces compaction of the autoinhibitory ARM domain and permits the formation of TIR-TIR interactions that activate SARM1. Activation of SARM1 may lead to neurodegeneration or neuronal dysfunction. Neurodegeneration refers to the loss of functional activity and trophic degeneration of the cell bodies of the neurons. Neuronal dysfunction may refer to the loss of functional activity and/or interruption in the transmission of nerve signals in the absence of gross morphological degeneration of axons/terminals or soma death.

Neurodegeneration may include axonal degeneration and/or neurolytic death. Axonal degeneration refers to the loss of functional activity and trophic degeneration of axons and their terminal arborizations. Neurolytic death or neurolysis refers to degeneration of part, or any combination thereof of the neuron or nerve cell, such as the cell body, axon, axon hillock, dendrites, terminal aborizations (telodendria), regenerated axons, regenerated terminal aborizations, synaptic terminals, including neuromuscular junctions and sensory receptors. When any part of thenerve cell degenerates, it causes an interruption in the transmission of nerve signals.

In respect of the SARM1 agents disclosed herein, whether for use in medical or non-therapeutic methods, the neurodegeneration (which may encompass axonal degeneration and/or neurolytic death) may be reversible by a reversal agent such as nicotinamide, NAD+ precursors such as nicotinic acid, NAMPT inhibitors such as FK866 and CHS-828 (also known as GMX1778), SARM1 inhibitors and NMNATs (including NMNAT1-2-3) activators, and NMNATs stabilisers which reduce NMNAT degradation/turnover. The reversal agent may be any agent that can reverse the effect of the SARM1 agent.

The neurodegeneration/degeneration may occur in (i) cells expressing SARM1. For example, in neurons. Neurons refers to a nerve cell, the principal functional units of the nervous system. A neuron consists of a cell body and its processes—an axon, one or more dendrites, its terminal aborizations or telodendria and synaptic terminals. Neurons transmit information to other neurons or cells by releasing neurotransmitters at synaptic terminals. Examples of neurons include dopaminergic neurons, GABAergic neurons, glutamate neurons, spinal cord neurons, for example, sensory neurons, nociceptive neurons, dorsal root ganglion neurons, pre- and post-ganglionic sympathetic and parasympathetic neurons, enteric neurons such as dogiel neurons, motor neurons, interneurons, unipolar cells, bipolar cells, multipolar cells, psuedounipolar cells, pyramidal cells, basket cells, stellate cells, purkinje cells, betz cells, amacrine cells, granule cell, ovoid cell, medium spiny neurons and large spiny neurons and all types of neurons.

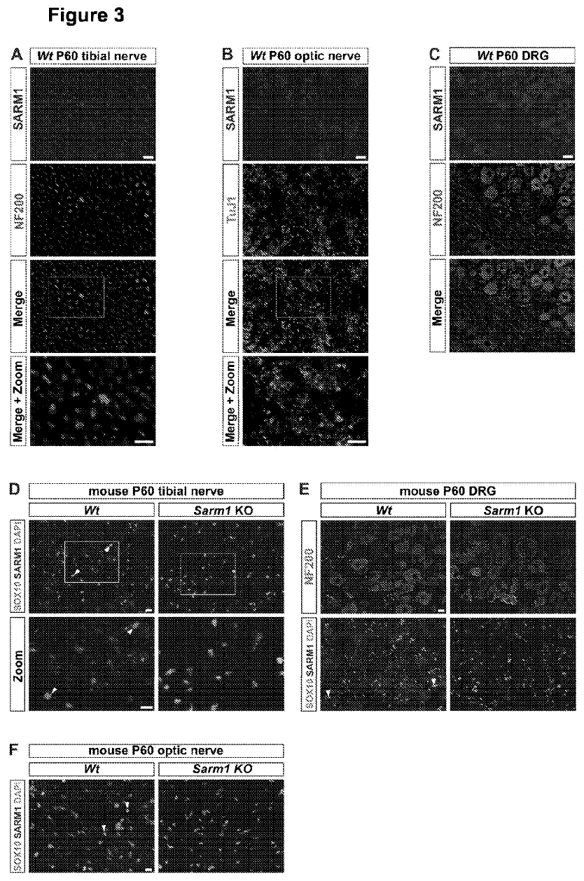

The neurodegeneration/degeneration may not occur in cells lacking or expressing low levels of SARM1. For example Schwann cells (including, myelinating, non-myelinating/Remak, perisynaptic/terminal, nociceptive, injury activated/Repair Schwann cells), muscle cells, skin cells (such as dermal fibroblasts), HEK293 cells, HEK293T cells, Hela cells, SK mel 2 cells, SK mel 5 cells, SK mel 28 cells, CAPAN cells, JURKAT cells, C6 cells, HL-60 cells, LP-1 cells, U937 cells, MEWO cells, monocytes/macrophages, and/or RAW264.7 cells (Uccellini et al., 2020; Buonvicino et al., 2018; Zhao et al, 2019) (FIGS. 3,5,7,15, 17, and 20). The neurodegeneration induced by SARM1 activation (e.g. by the SARM1 agents described herein) is selective for some cells making it appropriate for the medical and non-therapeutic methods disclosed herein. A low level of expression in some examples may be a functionally insignificant level. In some examples, functionally insignificant refers to a level of SARM1 that does not provide a level of SARM1 that can cause effects on the cell such as neurodegeneration and/or neuronal dysfunction. In some examples, functionally insignificant refers to an expression level that is below a detectable threshold by methods known in the art and described herein. In some examples, functionally insignificant may refer to a level of expression below a baseline level, for example, in comparison to one or more housekeeping or control genes. In some examples, techniques for detecting RNA (i.e., RT-PCR, PCR, hybridization) or proteins (i.e., ELISA, immunohistochemical techniques) may be used to determine an expression level of SARM1. In some examples, the Schwann cells may be injury activated Schwann cells (e.g. repair Schwann cells).

In some examples, after administration and neurodegeneration the neuronal cells may be able to regenerate. For example, the neurodegeneration may allow for neuro-regeneration. Without being bound by theory regeneration may occur due to the temporary and/or reversible nature of the mode of action of SARM1 agents. In addition, the insensitivity to SARM1 agents of Schwann cells (including terminal SC responsible for reinnervation of the neuromuscular junction and nociceptive Schwann cells associated with pain fibre nerve endings) may also allow for neuronal regeneration to occur. The occurrence of neurodegeneration may provide advantages in the treatment of certain conditions as well as cosmetics uses described herein as the SARM1 agents may not cause permanent neurodegeneration and possible paralysis (for example of facial muscles in cosmetic uses) that may lead to adverse side-effects or outcomes.

Neurodegeneration/degeneration can be measured using any method known in the art. For example, neurodegeneration can be measured quantitatively or qualitatively by one or more techniques selected from the group consisting of electroencephalogram (EEG), electromyogram (EMG), evoked potentials, neuroimaging, functional MM, structural Mill, diffusion tensor imaging (DTI), [18F]fluorodeoxyglucose (FDG) PET, agents that label amyloid, [18F] F-dopa PET, radiotracer imaging, volumetric analysis of regional tissue loss, specific imaging markers of abnormal protein deposition, multimodal imaging, microscopy of pathological specimens, and biomarker analysis (include clinical neurological motor sensory examination, sense of smell examination, cognitive testing, sleep studies, circadian rhythm analysis, serum/cerebral spinous fluid biomarkers).

Neuronal dysfunction can be determined using any means known in the art. Examples are studies of neuronal and/or neurite morphology using microscopy (Loreto et al. 2021 eLife), measuring levels of neurofilament light chain (NfL) in in vitro assay, (e.g. culture supernatants) serum and Cerebrospinal fluid (CSF) (see https://www.sciencedirect.com/science/article/pii/S2211124720315771?via % 3Dihub), metabolomics assays as described above, electrophysiology-nerve conduction studies/electromyography, mitochondrial dynamics and function, axonal transport, metabolomic changes, serum/CSF biomarkers, such as neurofilament. Motor/sensory neurological examination including MRC scales and sum score, cognitive testing, such as Addenbrookes cognitive exam III, sense of smell examination, sleep studies, circadian rhythm analysis, methods to measure peak volitional muscle contraction force, such as dynamometry and muscle fatigue, such as neurological examination and neurophysiology, timed walking tests, objective sensory examinations such as Von frey hair, thermal thresholds, proprioceptive tests, such as the 9 hole peg test. Intraepidermal nerve fibre analysis.

### Administration

The SARM1 agent can be administered by any suitable route including, for example, injection, intravenous infusion, intradermal, subcutaneous, percutaneous, intramuscular, intra-arterial, intraperitoneal, intraarticular, intraosseous, intravesicular, transdermally, orally, topically, or other appropriate administration routes. Administration can be “parenteral administration” meaning modes of administration other than enteral and topical administration, usually by injection. Alternatively, SARM1 agent(s) can be administered via a non-parenteral route, such as a topical, epidermal or mucosal route of administration. Local administration may also be carried out, including peritumoral, juxtatumoral, intratumoral, intralesional, perilesional, intra cavity infusion, intravesicle administration, and inhalation. In some examples, SARM1 agent(s) may be administered peripherally. For example, peripheral administration includes intravenous, intra-arterial, subcutaneous, intramuscular, intraperitoneal, transdermal, by inhalation, transbuccal, intranasal, rectal, oral, parenteral, sublingual, or trans-nasal. In preferred embodiments, the SARM1 agent disclosed herein is administered by a transdermal patch, a composition (such as an ointment or cream), or by a syringe such as a pre-filled syringe. Other routes that are useful are those that can be used to deliver SARM1 agent(s) into the skin or muscle.

Any suitable route of administration can be used to administer said composition including but not limited to: oral, aerosol or other device for delivery to the lungs, nasal spray, intravenous, intramuscular, intraperitoneal, intrathecal, vaginal, rectal, topical, lumbar puncture, intrathecal, and direct application to the brain and/or meninges.

In particular, the SARM1 agent disclosed herein may be administered locally. For example by a transdermal patch. In some examples by injection into the skin or muscle, including the anal sphinchter. In some examples, by intravesical injection into detrusor muscle/urethral sphincter, with or without ultrasound, radiographic and/or electromyographic guidance. In some examples, by endoscopic injection such as into the oesophageal sphincter, pyloric sphincter, sphincter of Oddi; cystoscopic injection into the urethral sphincter. In some examples, by injection into salivary glands such as sublingual and submandibular glands or into the axilla or groin.

Topical administration may be achieved by the use of a topical composition of a SARM1 agent as described herein. Topical composition refers to a composition suitable for application to mammalian, e.g., human, skin. Non-limiting examples of topical compositions include skin care formulations such as cleansers, toners, serums, sticks, wipes, masks, lotions, creams, ointments, balms, oils, scrubs, liquid, bar, gel, oil, foam or treatments; as well as cosmetic products, including, but not limited to, foundations, eye liners, eye shadows, blushes, bronzers, highlighters, lip liners, brow pencils, blemish/beauty balm creams, colour correcting/control creams, lipsticks, mascaras, lip glosses, lip balms, concealers, and powders.

In some examples, the topical composition is a cream or ointment. Cream refers to a soft, semi-solid, pharmaceutically and cosmetically acceptable preparation intended for external application to skin or other tissues. Creams typically include an aqueous base formulated as a water-in-oil emulsion or as an oil-in-water emulsion. Ointment refers to a homogeneous, viscous preparation that can be applied to a subject. Ointment can be considered synonymous to a lotion, a cream, an emulsion, a gel, or an emollient.

The SARM1 agent disclosed herein may also be administered into the scalp, gums, legs, calves, arms, armpits, palms, soles, groin and shoulders and any other body part. The administration may be performed via any means known in the art such as by injection. The SARM1 agent can be transdermally administered to a subject for treating conditions disclosed herein such as undesirable facial muscle or other muscular spasms, hyperhidrosis, acne, or conditions elsewhere in the body in which relief of muscular ache or spasms is desired. The SARM1 agent may be administered by injection or topically for transdermal delivery to muscles or to other skin-associated structures. The administration may be made, for example, to the legs, shoulders, back (including lower back), axilla, palms, feet, neck, groin, dorsa of the hands or feet, elbows, upper arms, knees, upper legs, buttocks, torso, pelvis, or any other part of the body where administration of the SARM1 agent is desired.

The SARM1 agent, compositions and formulations comprising a SARM1 agent may be applied so as to administer an effective amount of the SARM1 agent. The term “effective amount” as used herein means an amount of a SARM1 agent as defined above that is sufficient to produce the desired effect e.g., removal/prevention of wrinkles, to induce muscular paralysis or other biological or aesthetic effect as disclosed herein, but that implicitly is a safe amount, i.e. one that is low enough to avoid serious side effects. Desired effects may include the relaxation of certain muscles with the aim of, for instance, decreasing the appearance of fine lines and/or wrinkles, especially in the face, or adjusting facial appearance in other ways such as widening the eyes, lifting the corners of the mouth, or smoothing lines that fan out from the upper lip, or the general relief of muscular tension. The last-mentioned effect, general relief of muscular tension, can be affected in the face or elsewhere. An appropriate effective amount of the SARM1 agent for application may be a single-dose treatment, or may be more concentrated, either for dilution at the place of administration or for use in multiple applications.

The SARM1 agents disclosed herein may be co-administered. For example, more than one SARM1 agent may be used administered together as a medicament whether simultaneously or sequentially, e.g., in a method of treating any disease or condition as disclosed herein. For example, one SARM1 agent may be administered first and the second SARM1 agent may be administered up to 5 minutes, 10 minutes, 15 minutes, 20 minutes, 25 minutes, 30 minutes, 45 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 48 hours, 72 hours, 96 hours, 1 week, 2 weeks, 3 weeks, 4 weeks, 5 weeks, 6 weeks, 7 weeks 8 weeks, 3 months, 4 months, 5 months, 6 months, 7 months, 8 months, 9 months, 10 months, 11 months, 12 months, 2 years, 3 years, 4 years, or 5 years after the first SARM1 agent was administered. Repeat administration of the same SARM1 agent may also be performed.

The amount of the SARM1 agent (e.g., Vacor, metabolites, analogs and derivatives thereof) as disclosed herein required for use in treatment will of course vary not only with the particular compound but also with the route and form of administration, the nature and severity of the condition being treated, and the type, age and condition of the organism. Thus, appropriate concentrations of the SARM1 agent to be incorporated into compositions and formulations can be routinely determined by those skilled in the art in accordance with standard practices.

Any of the above-described SARM1 agent(s), and products containing SARM1 agent(s) may further comprise one or more additional agents. The one or more additional agents may be selected from the group consisting of botulinum toxin and capsaicin, local anaethestics, sympathomimetic drugs, steroid drugs, reversal agents and molecules that interfere with enzymes in the NAD biosynthetic pathway. Local anaesthetics may include lidocaine, bupivicaine; opiate analgesics such as morphine, fentanyl, and oxycodone. Sympathomimetic drugs may include adrenaline, and noradrenaline. Steroid drugs may include prednisolone, betamethasone, dexamethasone and hydrocortisone. Reversal agents may include direct SARM1 competitive/non-competitive inhibitors as well as indirect inhibitors such as nicotinamide. Molecules that interfere with enzymes in the NAD biosynthetic pathway may include NAMPT inhibitors, such as FK866, inhibitors of NMNATs (1-2-3) and products of SARM1 base exchange activity, such as VAD and others (Loreto et al., 2021, eLife; Shi et al., 2022 Mol Cell).

Any of the above-SARM1 agent(s) and additional agents may be co-administered or administered sequentially. For example, an analgesic may be coadministered with one or more SARM1 agent(s). As a further example, two or more SARM1 agents can be co-administered. As a further example, a reversal agent may be administered following administration of any one of more of the SARM-1 agent(s) disclosed herein.

### Transdermal Patch, Syringe, Composition

Disclosed herein are also products such as a transdermal patch comprising a SARM1 agent as disclosed herein, a syringe comprising a SARM1 agent as disclosed herein and compositions comprising a SARM1 agent as disclosed herein. Such products are useful for the delivery of a SARM1 agent to the subject.

The transdermal patch may allow one or more SARM1 agents to be delivered into the subject's system at a controlled rate through the skin. The SARM1 agents may be co-administered with any other agent such as drugs and the like. A patch or pump apparatus or other system may be placed into contact upon a region of skin to transport molecules such as SARM1 agents, other agents, or drugs and the like transdermally via a number of different mechanisms such as maintaining simple contact of molecules upon the skin surface for absorption (either with or without chemical penetration enhancers), iontophoresis, needles, in-dwelling catheters, etc. The patch or pump may be removably adhered or placed directly upon the user's skin surface via any number of methods such as being adhered directly to the skin via a temporary adhesive layer or optionally through direct pressure utilizing a strap or band.

The transdermal patch may allow the SARM1 agent and any other agent of choice to penetrate the skin surface by passive or active mechanisms. Examples of passive mechanisms may include simple diffusion or absorption through the skin, osmosis, etc., and some examples of active mechanisms may include introduction through the skin via mechanical insertion through needles, abrasion, etc., or through electrical methods such as iontophoresis where a suspension of the drug molecules is subjected to an electric field for passage into or through the adjacent skin surface and into the patient's blood stream.

For example, an iontophoresis-based patch uses an applied electrical current or voltage to drive pharmaceutical formulations through the skin. The electrical current can be programmed to adjust and control the rate of drug delivery into the patient skin. Other methods, such as the use of chemical penetration enhancers, heat, or ultrasound waves, may also be used to increase delivery rates through the skin barrier. Other patches or pumps include infusion pumps which deliver molecules via a needle or catheter inserted through the skin for conditions such as that used in insulin therapy for diabetes treatment. Such patches or pumps deliver molecules via a fluid vehicle where the fluid is typically retained within a reservoir which is coupled to a delivery mechanism such as a membrane, needle, or catheter depending upon the delivery mode.

The patch, pump or other system for delivery may include a number of features which facilitate molecule (SARM1 agent, other agent, drug etc.) delivery to the subject. For example, the patch or pump assembly may enclose or accommodate a fluid reservoir within a housing to contain the molecule or suspended in a fluid vehicle. The reservoir may be fluidly coupled via a microchannel lumen through which fluids or medications may be transported to a transdermal drug delivery mechanism in contact with or in proximity to the underlying skin surface. A pump may be used to drive or urge the molecules from the reservoir and through the skin via one of the drug delivery mechanisms, such as through an array of microneedles. The pump may also be used to deliver the drugs from the reservoir for placement directly upon the skin surface where it may be left in place or maintained in contact against the skin surface for absorption, e.g., by maintaining contact against the skin with a microporous membrane. In placing the molecules against the skin surface (rather than introducing or urging the molecules through the layer of skin), a number of chemical enhancing agents may be utilized to facilitate the absorption of the drugs into the skin surface. For instance, agents such as propylene glycol, ethyl alcohol, dimethyl sulfoxide, etc., may be placed upon the skin surface prior to, during, or after placement of the drugs upon the skin or such agents may be combined with the drugs in the fluid vehicle such that they are delivered along with the drugs directly upon the skin surface.

To control the pumping of the molecules as well as different treatment regiments, electronic control circuitry may also be positioned upon the housing and in electrical communication with the pump. A battery (that may be rechargeable or replaceable) may also be positioned along the housing for providing power to the pump, control circuitry, and any other features as necessary. For example, the electronic control circuitry may provide a variety of functionality and determines when the pump should be active. By controlling when the pump is active or inactive, the electronic circuitry may be used to control when fluid from the reservoir is pumped to the skin. The control circuitry may also include diagnostic algorithms and indicators, such as monitoring battery power, pump operation, circuit integrity, etc. Yet another element that may be included in control circuitry is the inclusion of an on-chip clock that tracks the time and date to facilitate regulation of the fluid delivery schedule controlled by the microprocessor, particularly for chronotherapeutic drug formulations where delivery of medication is on a timed schedule corresponding to the date and/or time of day. Additionally, or optionally, a flow rate monitor may also be included in the control circuitry to monitor the amount of fluid which has been delivered upon or through the skin. Further any microchannels fluidly coupling the various features, such as the reservoir, pump, and microporous membrane may be formed directly within or along the housing and sized to have cross-sectional dimensions ranging anywhere from 1 micron to 5000 microns and lengths varying anywhere from 1 millimeter to 1 meter or longer. Because of their size, the microchannels may facilitate the passage of fluids, such as through capillary action, such that fluid delivery through the channels is consistent regardless of the orientation or angle of the assembly. Moreover, the microchannels may also help to inhibit or prevent the formation of bubbles within the channels such that molecule delivery may be metered consistently to the patient.

Use of a microchannel as the molecule reservoir may also allow for different types of pump configurations. Rather than using a pump to extract liquid from the microchannel reservoir and pumping it out to ultimately reach a user's skin, an air or gas pump can instead be used to push the liquid down the length of the microchannel reservoir to be ultimately deposited on the user's skin.

The reservoir may be configured into various other patterns as well such as a spiral or any other configuration which allows for fluid storage in or along the housing. For example, the microchannel reservoir may be formed to have one or more separate channels which are aligned parallel to one another. Each of these separate parallel channels may converge into a single microchannel which is fluidly coupled to the pump or other mechanism. The number of channels and the lengths of the individual channels may be uniform or individually varied. Moreover, one or more of the microchannels may contain different formulations or varied dosages depending upon the resulting desired dosage and drug combination to be infused into the patient. Yet another variation of a microchannel reservoir may include, e.g., a first microchannel reservoir and a second microchannel reservoir which is separate and distinct from the first microchannel reservoir. Additionally, other variations may include one or more microchannel reservoirs which are aligned along multiple geometric planes within the housing. For instance, a first reservoir may be situated in a first plane along the housing while a second reservoir may be situated in a second plane below or above the first reservoir. In this manner, multiple reservoirs may be “stacked” atop or below one another in several adjacent planes which may be separate from one another, or which may be fluidly interconnected between two or more reservoirs between their respective planes. The microchannel reservoirs may each contain the same or different formulations or dosages and may each be coupled to one or more valves which may be electronically controlled to meter or control the volume of one or both reservoirs to be pumped. The microchannel reservoir may also be completely removable from the patch or pump. The microchannel reservoir can reside in a removable package or cartridge which may be inserted securely within an interface or receiving channel defined within the housing. Once the reservoir has been depleted, it may be refilled or the cartridge may be removed entirely from the housing and replaced with another cartridge without having to remove the housing from the patient's skin.

A programmable electronic circuitry of the patch or pump assembly may be included and equipped with a transmitter and/or receiver that allows it to communicate, wirelessly or otherwise, with an external controller such as a computer or hand-held device. The external controller can be used by a physician or the patient to program parameters such as drug delivery time-profiles for a particular patient, customising the delivery rate profile to a particular patient's needs for a particular medication, etc. Another aspect of the transdermal patch or pump assembly may provide the capability for patient-determined “on-demand” controlled delivery of medication. User-initiated responses may be used as signals to the programmable electronic circuitry to indicate the appropriate dosage profile to be used from that point forward or until a new user-initiated signal is received. These signals may also be used, for example, to initiate an “on-demand” bolus for drug delivery when the user of the patch or pump so desires. The amount and rate of molecule delivery when the “on-demand” button is pushed may be pre-determined by the circuitry. When a patient desires a small dose of the medication to be administered (such as may the case for an analgesic to relieve pain or a stimulant to help maintain awareness and remain alert), the patient may actuate a control such as pushing a button on the transdermal patch or pump to release a pre-set bolus of the medication/formulation. The control may be part of the electronic control itself.

The transdermal patch may be constructed according to any transdermal patch known in the art. For example, the transdermal patch for the application of the molecules disclosed herein (e.g., SARM1 agent, other agents or drugs) to the skin of a patient may comprise a backing layer, a liner layer, and a molecule-containing adhesive layer disposed between the backing layer and the liner layer. The molecule-containing adhesive layer may include polyisobutylene, a plasticizer for polyisobutylene, in which the ratio of the plasticizer and the polyisobutylene may be less than about 0.8, at least 5% by weight of a filler, and a molecule component comprising the molecule to be delivered which may be moderately soluble in the plasticizer. Preferably, the ratio of the plasticizer to the polyisobutylene is between about 0.05 and 0.8.

Relating to the transdermal patch, the molecule-containing adhesive layer may be in direct contact with the liner layer, whereby upon removal of the liner layer and application of the transdermal patch to the skin, the adhesive layer is in direct contact with the skin. The filler of the transdermal patch may be a metal oxide, an inorganic salt, a polymeric filler, a clay component, and/or a mixture thereof. Preferably, the metal oxide can be zinc, magnesium, calcium, or titanium oxide. The inorganic salts may include calcium, magnesium and sodium carbonates, calcium and magnesium sulfates, and calcium phosphate. The clay components may include talc, kaolin, and bentonite. The polyisobutylene of the transdermal patch may comprise a mixture of high molecular weight polyisobutylene and low molecular weight polyisobutylene. Preferably, the high molecular weight polyisobutylene has a viscosity average molecular weight of between about 450,000 and 2,100,000. Preferably, the low molecular weight polyisobutylene has an average molecular weight of between about 1,000 and 450,000. Preferably, the ratio of high molecular weight polyisobutylene:low molecular weight polyisobutylene is between about 20:80 and 70:30.

The transdermal patch may be used to deliver one or more molecules, such as one or more SARM1 agents, other agents such as drugs and the like including additional agents disclosed herein. Any drug/agent component of the transdermal patch may include additional agents disclosed herein such as local anaethestics, sympathomimetic drugs, steroid drugs, reversal agents and molecules that interfere with enzymes in the NAD biosynthetic pathway. Local anaesthetics may include lidocaine, bupivicaine; opiate analgesics such as morphine, fentanyl, and oxycodone. Sympathomimetic drugs may include adrenaline, and noradrenaline. Steroid drugs may include prednisolone, betamethasone, dexamethasone and hydrocortisone. Reversal agents may include direct SARM1 competitive/non-competitive inhibitors as well as indirect inhibitors such as nicotinamide. Molecules that interfere with enzymes in the NAD biosynthetic pathway may include NAMPT inhibitors, such as FK866, inhibitors of NMNATs (1-2-3) and products of SARM1 base exchange activity, such as VAD and others (Loreto et al., 2021, eLife; Shi et al., 2022 Mol Cell).

The plasticizer of the transdermal patch may comprise a hydrophobic liquid, for example, having a solubility parameter of between about 12 and 18 (J/cm3)1/2. The plasticizer may include mineral oil, linseed oil, octyl palmitate, squalene, squalane, silicone oil, isobutyl myristate, isostearyl alcohol, and oleyl alcohol, and the like. The mineral oil may be present in an amount of between about 10 and 40 weight percent.

In a particular embodiment, the transdermal patch may comprise a backing layer, a liner layer, and a SARM1 agent-containing adhesive layer disposed between the backing layer and the liner layer, the SARM-1 containing adhesive layer including polyisobutylene, a plasticizer comprising mineral oil for the polyisobutylene, the ratio of the mineral oil to the polyisobutylene being between about 0.5 and 0.8, and at least 5% by weight of a filler.

A syringe disclosed herein may comprise one or more molecules such as one or more SARM1 agent, other agents such as drugs and the like including additional agents disclosed herein. The syringe may be any syringe known in the art. For example, any drug/agent component of the syringe may include additional agents such as local anaethestics, sympathomimetic drugs, steroid drugs, reversal agents and molecules that interfere with enzymes in the NAD biosynthetic pathway. Local anaesthetics may include lidocaine, bupivicaine; opiate analgesics such as morphine, fentanyl, and oxycodone. Sympathomimetic drugs may include adrenaline, and noradrenaline. Steroid drugs may include prednisolone, betamethasone, dexamethasone and hydrocortisone. Reversal agents may include direct SARM1 competitive/non-competitive inhibitors as well as indirect inhibitors such as nicotinamide. Molecules that interfere with enzymes in the NAD biosynthetic pathway may include NAMPT inhibitors, such as FK866, inhibitors of NMNATs (1-2-3) and products of SARM1 base exchange activity, such as VAD and others (Loreto et al., 2021, eLife; Shi et al., 2022 Mol Cell).

The syringe may be a pre-filled syringe for convenient administration. Such syringes where the syringe barrel of an injection syringe is filled with a drug in advance are known in the art. Such prefilled syringes are equipped with a nozzle for fitting an injection needle at one end of a syringe barrel constituting a body of the syringe. At the other end, an opening is provided where a plunger having a gasket at a tip of a plunger rod can be freely moved backwards and forwards. After filling with a drug, the nozzle is usually covered over with a cap made of rubber in order to prevent leakage of the drug. The entire item is then blister-packed or pillow-packed with the plunger installed. Injection syringes prefilled with a drug where a nozzle covered with a cap is then covered with a heat-shrinkable resin film (shrink film) in order to prevent the cap from coming off as well as in order to prevent the filled drug from being tampered with are also known. When the syringe is made of plastic, it may be necessary to store the syringe in barrier packing material having gas barrier properties in order to suppress deterioration of the drug filling the syringe barrel. If the syringe is stored in a barrier packing material, the drug filling the syringe barrel does not degrade because of the gas barrier properties.

The composition disclosed herein may be of any formulation known in the art. The composition may be applied topically for the delivery of one or more molecules, such as one or more SARM1 agents, other agents such as drugs and the like. For example, the composition disclosed herein may comprise solutions, emulsions (including microemulsions), suspensions, creams, lotions, gels, powders, or other typical solid or liquid compositions used for application to skin and other tissues where molecules (e.g., SARM1 agent, and/or other agents) may be used. For example, composition may be of a formulation that is designed for efficient topical application onto the skin. Such compositions may contain, in addition to the SARM1 agent and any carrier, other ingredients typically used in such products, such as antimicrobials, moisturizers and hydration agents, penetration agents, preservatives, emulsifiers, natural or synthetic oils, solvents, surfactants, detergents, emollients, antioxidants, fragrances, fillers, thickeners, waxes, odor absorbers, dyestuffs, coloring agents, powders, and optionally including anesthetics, anti-itch additives, botanical extracts, conditioning agents, darkening or lightening agents, glitter, humectants, mica, minerals, polyphenols, silicones or derivatives thereof, sunblocks, vitamins, and phytomedicinals. The compositions may also include gelling agents and/or viscosity-modifying agents. These agents are generally added to increase the viscosity of the composition, so as to make the application of the composition easier and more accurate. Additionally, these agents help to prevent the aqueous SARM1 agent/carrier solution from drying out, which tends to cause a decrease in the activity of the SARM1 agent. Particularly preferred agents are those that are of a charge that does not interfere with the SARM1 agent activity or the efficiency of the neurotoxin-carrier complexes in crossing the skin. The gelling agents may be certain cellulose-based gelling agents, such as hydroxypropylcellulose (HPC) for example. In some embodiments, the SARM1 agent/carrier complex is formulated in a composition having 2-4% HPC. Alternatively, the viscosity of a solution containing a SARM1 agent/carrier complex may be altered by adding polyethylene glycol (PEG). In other embodiments, the SARM1 agent/carrier solution is combined with pre-mixed viscous agents, such as CETAPHIL® moisturizer.

The compositions may optionally include partitioning agents. As used herein, a “partitioning agent” is any substance or additive that has the property of preventing or minimizing unwanted or uncontrolled aggregation of SARM1 agents and any molecules to be delivered with the carriers of this invention. Partitioning agents may be useful, for example, when a concentrated solution comprising SARM1 agents must be employed due to volume constraints. In these cases, the partitioning agent keeps the SARM1 agent dispersed, thereby preventing aggregation that would otherwise occur without the partitioning agent. Generally, a partitioning agent is (1) non-irritating, (2) does not destroy the active agent e.g., SARM1, (3) does not confer any increase in permeability, (4) affords reliable and stable particle sizes, (5) is of the relevant charge, and (6) does not interfere with complexes of the neurotoxin and the transdermal carrier. An example of a suitable partitioning agent is ethanol (EtOH). In preferred embodiments, the EtOH is less than 20% of the composition, and most preferably, less than 5% of the composition.

An oligo- or polyanion bridge may be added to the SARM1 agent so to increase the efficiency and efficacy of topical administration. Examples of such oligo-/polyanion bridges include sodium phosphate (5%), PBS, or 5% poly-L-aspartate (e.g., with a MW of 3000).

Compositions disclosed herein may be in the form of controlled-release or sustained-release compositions, wherein the SARM1 agent and any carrier are encapsulated or otherwise contained within a material such that they are released onto the skin in a controlled manner over time. The SARM1 agent and any carrier may be contained within matrixes, liposomes, vesicles, microcapsules, microspheres and the like, or within a solid particulate material, all of which is selected and/or constructed to provide release of the SARM1 agent over time. The SARM1 agent and any carrier may be encapsulated together (e.g., in the same capsule) or separately (in separate capsules).

The compositions described herein can thus be used to deliver molecules (e.g., SARM1 and any carrier, agent etc.) to muscles underlying the skin, or to glandular structures within the skin, in an effective amount to produce paralysis, produce relaxation, alleviate contractions, prevent or alleviate spasms, reduce glandular output, or other desired effects. Local delivery of the SARM1 agent in this manner could afford dosage reductions, reduce toxicity and allow more precise dosage optimization for desired effects relative to injectable or implantable materials.

### Kits

The SARM1 agents or compositions thereof may be provided as a kit of parts. For example, the kit may include a SARM1 agent or composition thereof as described herein and an applicator for administration of the SARM1 agent to a subject. For example, an applicator may be a syringe or transdermal patch as described herein. In some examples, the SARM1 agent or composition thereof may be in a unit dose form.

The kit may also include instructions for use.

### Other Definitions

The subject may be referred to herein as a patient. The terms “subject”, “individual”, and “patient” are used herein interchangeably. As used herein, the term “subject” is intended to include humans and animals. Typically, a subject refers to a human or animal that, within their body, has a cell deficient in homologous recombination. Examples of subjects include mammals, e.g., humans, dogs, cows, horses, pigs, sheep, goats, cats, mice, rabbits, rats, and transgenic non-human animals. In some examples, subjects include companion animals, e.g. dogs, cats, rabbits, and rats. In some examples, subjects include livestock, e.g., cows, pigs, sheep, goats, and rabbits. In some examples, subjects include thoroughbred or show animals, e.g. horses, pigs, cows, and rabbits.

Amino acid identity may be calculated using any suitable algorithm. For example the PILEUP and BLAST algorithms can be used to calculate identity or line up sequences (such as identifying equivalent or corresponding sequences (typically on their default settings), for example as described in Altschul S. F., 1993 J Mol Evol 36:290-300; Altschul, S, F et al., 1990 J Mol Biol 215:403-10. Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al., supra). These initial neighborhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff, 1992 Proc. Natl. Acad. Sci. USA 89:10915-10919) alignments (B) of 50, expectation (E) of 10, M=5, N=4, and a comparison of both strands.

The BLAST algorithm performs a statistical analysis of the similarity between two sequences; see e.g., Karlin and Altschul, 1993 Proc. Natl. Acad. Sci. USA 90:5873-5787. One measure of similarity provided by the BLAST algorithm is the smallest sum probability (P(N)), which provides an indication of the probability by which a match between two polynucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001. Alternatively, the UWGCG Package provides the BESTFIT program which can be used to calculate identity (for example used on its default settings) (Devereux et al., 1984 Nucleic Acids Research 12, 387-395).

The amino acid sequence of any biological molecules referenced herein such as SARM1 may comprise the amino acid sequence of the whole or a portion of a reference sequence defined by SEQ ID NOs disclosed herein in which modifications, such as amino acid additions, deletions or substitutions are made relative to the reference sequence or portion thereof. The modifications may be conservative or non-conservative amino acid substitutions. Where there are multiple modifications in a single polypeptide, the modifications in the polypeptide sequence may be a combination of conservative and non-conservative amino acid substitutions. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid.

### EXAMPLES

The following Examples are provided to illustrate the invention, but not to limit the invention.

### Example 1—Sarm1/SARM1 Detection in Schwann Cells and Oligodendrocytes Though Deletion Doesn't Perturb Myelination in Zebrafish and Mice

SARM1 is central regulator of programmed axon death and is required to initiate axon self-destruction after traumatic and toxic insults to the nervous system. Abnormal activation of this axon degeneration pathway is increasingly recognised as a contributor to human neurological disease and SARM1 knockdown or inhibition has become an attractive therapeutic strategy to preserve axon loss in a variety of disorders of the peripheral and central nervous system. Despite this, it remains unknown whether Sarm1/SARM1 is present in myelinating glia and whether it plays a role in myelination in the PNS or CNS. It is important to answer these questions to understand whether future therapies inhibiting SARM1 function may have unintended deleterious impacts on myelination. This example shows that Sarm1 mRNA is identifiable in PNS and CNS glia in zebrafish and mice. SARM1 protein is present in oligodendrocytes, but not in myelinating and Remak Schwann cells and satellite glia in the adult murine nervous system. Use of specific SARM1 activators in cultured cells confirms that Schwann cells contain negligible functional SARM1 whereas oligodendrocytes in vitro are sensitive to endogenous SARM1 activation. Additionally, using zebrafish and mouse Sarm1 mutants, this example shows that SARM1 is not required for correct initiation of myelination nor myelin sheath maintenance by oligodendrocytes and Schwann cells. Thus, strategies to inhibit SARM1 function in the nervous system to treat neurological disease are unlikely to perturb myelination in humans.

The programmed axonal death (also termed Wallerian degeneration) pathway is becoming increasingly linked to neurological disease. Two of the most important regulators of this pathway are nicotinamide mononucleotide adenylyltransferase 2 (NMNAT2) and Sterile-α and Toll/interleukin 1receptor (TIR) motif containing protein 1 (SARM1), a member of the MyoD88 family (Gilley and Coleman, 2010; Osterloh et al., 2012; Gerdts et al., 2013; Coleman and Hoke, 2020). Complete loss of function mutations in Nmnat2 have been implicated in cases of fetal akinesia deformation sequence (FADS), where foetuses are stillborn with severe skeletal hypoplasia and hydrops fetalis, whereas partial loss of function mutations have been linked with the development of peripheral neuropathy with erythromelalgia (Huppke et al., 2019; Lukacs et al., 2019). SARM1 function is conserved in humans and the SARM1 locus has been identified in two Genome-wide association studies (GWAS) for Amyotrophic lateral sclerosis (ALS) (Fogh et al., 2014; van Rheenen et al., 2016; Chen et al., 2021). Following this, gain of function SARM1 mutations have been identified in sporadic ALS and other motor nerve disorders (Gilley et al., 2021; Bloom et al., 2022). Additionally, the disused rat poison, vacor, which leads to highly specific activation of SARM1, causes severe neurotoxic effects in humans. (LeWitt, 1980; Loreto et al., 2021).

Axon dysfunction and loss are hallmarks of many neurological diseases of the central (CNS) and peripheral nervous system (PNS), including Parkinson disease, traumatic brain injury, progressive multiple sclerosis, ALS and the many inherited and acquired peripheral neuropathies (Coleman and Höke, 2020). Sarm1 deletion. has shown to have significant protective effects on axon loss in animals models of traumatic brain injury, metabolic neuropathy and several models of chemotherapy induced neuropathy (Geisler et al., 2016; Henninger et al., 2016; Turkiew et al., 2017; Cheng et al., 2019; Geisler et al., 2019a; Marion et al., 2019; Bosanac et al., 2021; Gould et al., 2021b). Thus, inhibition or knockdown of SARM1 through use of pharmacological, gene therapy and antisense oligonucleotide approaches have become very attractive therapeutic strategies to trial in various neurological diseases (Geisler et al., 2019b; Coleman and Höke, 2020; Krauss et al., 2020; Arthur-Farraj and Coleman, 2021; Bosanac et al., 2021; Gould et al., 2021a; Merlini et al., 2022).

SARM1 is abundant in the nervous system but is not found in most other tissues, including heart, kidney, liver, lung, skeletal muscle, spleen or thymus. (Kim et al., 2007; Chen et al., 2011). While neurons have been shown to have high levels of SARM1 it is unknown whether it is also present in myelinating glia in the PNS or CNS. Furthermore, it remains undetermined, due to the lack of in-depth quantitative studies on myelination, whether SARM1 has any role in regulating oligodendrocyte or Schwann cell myelination or myelin maintenance in either a cell autonomous or non-cell autonomous fashion. This is important to know as it could have adverse implications for the use of therapies aimed to treat various neurological disorders through inhibition of SARM1 function.

In this example, mice and larval zebrafish were used to demonstrate that SARM1/sarm1 mRNA is present at low levels in developing Schwann cells and oligodendrocytes, whereas SARM1 protein is only detectable in oligodendrocytes but not Schwann cells in cell culture and in the adult murine nervous system. In cultured cells, use of the specific SARM1 activators, vacor and 3-acetylpyridine (3-AP) demonstrated that Schwann cells contain insignificant amounts of SARM1 whereas cultured oligodendrocytes contain functionally relevant levels of SARM1 protein. Furthermore, it is show that, in the absence of SARM1/Sarm1, myelination in the PNS and CNS is initiated normally in both mice and larval zebrafish and that PNS and CNS myelin maintenance in adult mice is unaffected.

### Materials and Methods

### Animals

All zebrafish research complied with the Animals (Scientific Procedures) Act 1986 and the University of Cambridge Animal Welfare and Ethical Review Body (AWERB), project license code P98A03BF9. Zebrafish (Danio rerio) were maintained at 28° C. Tg[mbp:eGFPCAAX] fish were a gift from Dave Lyons (Almeida et al., 2011), and Sarm111193 mutant fish were obtained from ZIRC (Anon) (Kettleborough et al., 2013). Tg[mbp:eGFPCAAX] and sarm1SA11193 embryos were obtained by natural spawning and raised in E3 medium (5 mM NaCl, 0.17 mM KCl, 0.33 mM CaCl2, 0.3 mM MgSO4, and 0.1% methylene blue) in Petri dishes until 24 hours post fertilisation. Embryos were then incubated in E3 medium supplemented with 0.003% phenylthiourea (PTU) to prevent pigmentation. F2 generation fish were used for analysis. sarm1+/+ (wt) were compared to sarm1SA11193/SA11193 (sarm1 mutant) for all experiments. Mouse research complied with European Union guidelines and protocols were approved by the Comité de Bioética y Bioseguridad del Instituto de Neurociencias de Alicante, Universidad Miguel Hernández de Elche and Consejo Superior de Investigaciones Científicas (http://in.umh-csic.es/), Reference number 2017/VSC/PEA/00022 tipo 2. Sarm1 knockout mice on C57BL/6J background (Kim et al., 2007) were bred from heterozygote crosses and F1 littermates were used for analysis. Mice were genotyped as previously described (Kim et al., 2007).

### Genotyping Zebrafish

Zebrafish genomic DNA was isolated from adult zebrafish fins and incubated at 55° C. overnight with 0.5 mg/ml Proteinase K in a 10 mM Tris-HCL buffer containing 50 mM KCL and 10% Tween20 and 10% NP40. PCR was run using primers, Forward: 5′-TCTGGAGCTGGTGGAGCCCT-3′ (SEQ ID NO: 1); Reverse: 5′-AGTCTAGTTTCTGCCTGACCTTGG-3′ (SEQ ID NO: 2). PCR product was then restriction enzyme digested at 37° C. for 1 hour with Msel (New England Biolabs) and then run on a 1.5% Agarose gel. WT PCR product produces a band of 233 base pairs. Mutant PCR product produces two bands of 165 and 68 base pairs.

### Plasmids and Microinjection

To generate the neuroD:tdTomato construct, the p-5E neurod5 kb promotor entry vector (Mo and Nicolson, 2011), gifted by Dr A. Nechiporuk. was inserted along with pME tdTomato (Oehlers et al., 2015), gifted by Dr D. Tobin (Addgene plasmid #135202) and p3EpolyA into pDestTol2pA2 using the Gateway™ system as previously described (Kwan et al., 2007). To visualise posterior lateral line neurons, 20 pg of the neuroD:tdTomato construct were microinjected into one cell stage embryos. Larvae were then fluorescently sorted at 4dpf.

### 2-Photon Axotomy of PLL Axons in Larval Zebrafish

Axotomies were carried out at the Cambridge Advanced Imaging Centre on a TriM Scope II two-photon Scanning Fluorescence Microscope using Imspector Pro software (LaVision Biotec) and a near-infrared laser source (Insight DeepSee, Spectra-Physics). The laser light was focussed by a 25×, 1.05 Numerical Aperture water immersion objective lens (XLPLN25XWMP2, Olympus). Axotomies were carried out by focusing the laser at a 6×6 μm section of the posterior lateral line using 100% laser power.

### Schwann Cell Culture

Freshly plated mouse Schwann cells were obtained from P2 sciatic and brachial nerves. Nerves were digested with trypsin and collagenase, centrifuged and plated on laminin/Poly-L-lysine coated glass coverslips in defined medium as previously described (Arthur-Farraj et al., 2011).

### Measurement of Intracellular NAD⁺

Schwann and HEK 293T cells were treated with 100 μM vacor, 250 μM 3AP, or vehicle controls, and collected in media at Oh and 72 h after treatment. Protein was extracted using Pierce™ IP Lysis Buffer supplemented with cOmplete™ Mini EDTA-free Protease Inhibitor Cocktail, and a BCA assay carried out to determine protein concentration. All samples were diluted to 0.5 μg/μL. The NAD-glo assay was performed according to manufacturer's instruction of the NAD⁺/NADH-Glo™ assay by Promega (G9071). In short, 25 μL of sample were incubated with 12.5 μL 0.4M HCl at 60° C. for 15 min, before 12.5 μL 0.5M Tris base were added. 10 μL NAD⁺ standards or sample were then mixed with 10 μL NAD-glo master mix (1 ml luciferin detection reagent, 5 μl reductase, 5 μl reductase substrate, 5 μl NAD⁺ cycling enzyme, 25 μl NAD⁺ cycling substrate) and incubated at RT for 40 min. Luminescence was read using a GloMax® Explorer microplate reader and NAD⁺ concentrations determined relative to a NAD⁺ standard curve.

### Oligodendrocyte Cell Culture

Sprague-Dawley neonatal (sp7) rat pups were decapitated following lethal overdose with pentobarbital. The brains were dissected and submerged into Hibernate-A low fluorescence media (Transnetyx Tissue, #HALF). The tissue was cut into 1-mm³ pieces, washed in HBSS^{-/-} (Gibco, #11039047), then spun down at 100 g for 1 min at room temperature. To digest the tissue, the tissue was incubated in 34 U/mL papain (Worthington, #LS003127) and 40 μg/mL DNase I (Sigma, #D5025) in HALF, and incubated on an orbital shaker (55 rpm) for 40 min at 37° C. To obtain a single-cell suspension, the tissue was triturated in HALF supplemented with 2% B27 and 2 mM sodium pyruvate, first using a 5-ml serological pipette and then three fire-polished glass pipettes of descending diameter. The supernatant containing the cells was filtered through 70-μm strainers into a tube containing 90% isotonic Percoll (GE Healthcare, #17-0891-01, in 10×PBS pH 7.2 (Gibco, #70013032). The final volume was topped up with DMEM/F12 (Gibco, #31331028) then inverted several times to yield a homogenous suspension with a final Percoll concentration of 22.5%. The single cell suspension was then separated from tissue and myelin debris by gradient density centrifugation at 800 g (without brakes) for 20 mins at room temperature. The myelin debris and supernatant were aspirated, leaving only the cell pellet, which was then resuspended in HBSS^{-/-} to wash out the Percoll. Subsequently, red blood cell lysis buffer (BD Biosciences, #555899) was used to remove red blood cells. OPCs were isolated by positive selection using 2.5 μg A2B5 (Merck Millipore, #MAB312) primary antibody, followed by 20 μl of rat anti-mouse IgM antibody (Miltenyi, #130-047-302) per brain using the MACS protocol according to the manufacturer's instructions. To collect the A2B5+ fraction, the MACS MS column (Miltenyi, #130-042-201) was removed from the magnetic stand (Miltenyi, #130-042-102) and cells were flushed from the column with 1 ml of prewarmed OPC medium (DMEM F/12 containing N-Acetyl cysteine (60 μg/ml, Sigma, #A9165), human recombinant insulin (10 ug/ml), sodium pyruvate (1 mM, Thermo Fisher, #11360-070), apo-transferrin (50 μg/ml, Sigma, #T2036), putrescine (16.1 μg/ml, Sigma, #P7505), sodium selenite (40 ng/ml, Sigma, #S5261), progesterone (60 ng/ml, Sigma, #P0130), bovine serum albumin (330 μg/ml, Sigma, #A4919). Cells were counted, then seeded at a density of 20,000 cells/cm² on poly-D-lysine (5 μg/ml PDL, Sigma #P6407) coated plates.

Freshly isolated OPCs were cultured in OPC medium containing proliferation factors, b-FGF (30 ng/ml, Peprotech, #100-18B) and PDGF (30 ng/ml, Peprotech, #100-13a) then changed into OPC media containing T3 (Triiodothyronine, Sigma, #T2877) for 5-7 days to induce OPC to oligodendrocyte differentiation. Whilst in culture, cells were maintained in a humidified incubator at 37° C., 5% CO2, 5% O2, with media changes every 48 hrs.

### Quantification of Vacor Treated Oligodendrocyte Cultures

Rat oligodendrocyte cultures were fixed at various time points following DMSO and Vacor treatment. These cells were immunofluorescently labelled with DAPI and an antibody to SOX10. Three representative images taken at 20× across cultures at different time points were quantified using Fiji for the presence of SOX10 expression. This experiment was repeated three times and average numbers of SOX10 positive oligodendrocytes counted per frame were represented.

### In Situ Hybridization Chain Reaction (HCR)

Embryos were fixed using 4% paraformaldehyde in calcium and magnesium free phosphate buffered saline (PBS) and stored at −20C in 100% MeOH. HCR 3.0 was performed as described in (Choi et al., 2018). In short, 2 pmol HCR probes were hybridised at 37° C. overnight in hybridisation buffer, followed by repeated washing in wash buffer. Probes were detected using 30 pmol fluorescent hairpins in amplification buffer overnight at room temperature followed by washing in 5×SSC 0.001% Tween-20. Samples were finally counterstained using 1 ug/ml DAPI and mounted in 80% glycerol in a MatTek glass bottom dish. DNA probes for zebrafish mbp, sarm1, sox10, fluorescent hairpins and buffers were purchased from Molecular Instruments, Inc.

### Imaging and Image Analysis

All fish were imaged on a Zeiss LSM 700 confocal microscope with a 40× oil objective. For imaging of living larvae, 4 or 5 days post fertilisation animals were anaesthetised in 0.01% tricaine (w/v) and embedded in 1% low melting point agarose (w/V in E3 media supplemented with 0.003% of PTU). For imaging of Tg[mbp:eGFPCAAX] zebrafish, images of the spinal cord and PLLn were obtained at the level of the first, third and seventh motor nerves distal to the urogenital opening. For live imaging after PLLn injury, images were taken every 10 minutes for up to 26 hours and embryos maintained at 28° C. Survival of fish was monitored by observing heartbeat and circulation. Confocal images are displayed as grid-stitched maximum intensity protections produced using Fiji (Preibisch et al., 2009; Schindelin et al., 2012). For fluorescence intensity measurements of Tg (mbp:eGFPCAAX) fish, integrated densities were obtained from regions of interest. For mbp HCR quantification, images of the PLLn and spinal cord were obtained at the level of the first, third and seventh motor nerves distal to the urogenital opening. For statistics coding of colour by the intensity of the mbp signal, nuclear segmentation was conducted in Imaris (Bitplane) using a surface mask around the DAPI stain with a surface detail of 0.22 μm, and touching surfaces were split using a seed size of 4 μm. For signal intensity analysis, surfaces for the spinal cord and posterior lateral line were drawn in Imaris (Bitplane) and intensity sum of both mbp and DAPI signals determined. Intensity was then normalised by the area of the surface. For all experiments between seven to nine fish were imaged (spinal cord and one PLLn) per genotype.

### Antibodies

Immunofluorescence: SOX10 (R&D Systems, 1:100, AF2864, RRID: AB_442208) donkey anti-goat IgG (H+L) Alexa Fluor 488 (Invitrogen, 1:1000, A11057), Anti-Beta III Tubulin (Sigma Aldrich, 1:1000, AB9354, RRID:AB_570918), NF200 (Abcam, 1:1000, ab72997, RRID: AB_1267598), SARM1 rabbit polyclonal antibodies (kind gift from Professor Hsueh, 1:500) and purchased from Abcam (Ab226930, 1:2000, RRID: AB_2893433), donkey anti-goat IgG (H+L) Alexa Fluor 488 (Invitrogen, 1:1000, A11057), Cy3 donkey anti-rabbit IgG (H+L) (Jackson Immunoresearch, 1:500, 711-165-152), Donkey anti-Mouse IgG (H+L) Highly Cross-Adsorbed Seco Alexa Fluor 488 (Invitrogen, 1:1000, A-21202), Donkey anti-Chicken IgY, Alexa Fluor 647 (Merck, 1:1000, 15389818), DAPI (Thermo scientific, 1:2000, 62248).

Western blot: CALNEXIN (Enzo Life Sciences, 1:1000, ADI-SPA-860-D, RRID: AB_312058), JUN (Cell Signalling Technology, 1:1000, 9165, RRID: AB_2130165), EGR2 (EMD Millipore, 1:500, ABE1374, RRID:AB_2715555), MPZ (Aves Labs, 1:2000, PZO, RRID:AB_2313561), MBP (EMD Millipore, 1:1000, AB9348, RRID: AB_2140366), SARM1 rabbit polyclonal and mouse monoclonal (kind gift from Professor Hsueh, 1:500), beta actin (Santa Cruz, 1:3000, sc-47778 HRP, RRID: AB_2714189), Anti-mouse IgG HRP-linked antibody (Cell Signalling Technology, 1:2000, 7076S), Anti-rabbit HRP-linked antibody (Cell Signalling Technology, 1:2000, 7074S), Goat pAb to Chicken IgY H+L (HRP) (Abcam, 1:2000, ab97135).

### Immunofluorescence

Sciatic and optic nerves were dissected from Wt and Sarm1 KO mice and directly embedded in O.C.T. compound, without fixing, and stored at −80° C. until ready for cryosectioning. Cryosections at 5 μm thick were collected onto SuperFrost plus slides. These slides were post-fixed in 4% paraformaldehyde (PFA) at room temperature for 10 mins followed by three washes of 5 mins each with 1×PBS. The slides were then immersed in 100% methanol at −20° C. for 10 mins, followed by three washes of 5 mins each with 1×PBS. The slides were then blocked in 5% horse serum (HS)/0.2% Triton x-100/PBS for 30 mins at room temperature. Primary antibodies were diluted in blocking solution and slides were incubated overnight at 4° C. The following day, the slides were washed once for 5 mins with 1×PBS, twice for 5 mins with 0.1% tween/PBS and finally once again for 5 mins with 1×PBS before adding secondary antibodies. The corresponding secondary antibodies were diluted at 1:500 along with DAPI at 1:2000 for 2 hrs at room temperature and kept in the dark. Slides were then washed once for 5 mins with 1×PBS, twice for 5 mins with 0.1% tween/PBS and finally once again for 5 mins with 1×PBS before mounting using Fluorsave (Calbiochem, 345789) and representative images were taken using Leica DMI6000B at 40×, 63× and 100×. The same protocol was followed for immunofluorescence staining in rat oligodendrocyte cultures and representative images were taken at 20× using Leica DMI8.

### Western Blot

Homogenates were obtained from sciatic or optic nerves. These lysates were prepared as described previously (Gomez-Sanchez et al., 2015). Samples were homogenized in ice-cold RIPA buffer with Halt™ Protease Inhibitor Cocktail (100×) (ThermoFisher Scientific 78429). After homogenization, the samples were incubated on ice for 30 min for further lysis. The resulting lysate was centrifuged at 12000 rpm. at 4° C., and the supernatant was taken for the Bicinchoninic acid (BCA) assay (ThermoFisher, 23227). The supernatant was diluted in loading buffer and boiled at 95° C. for 5 min. For western blot analysis of SARM1, 20 ug of protein was loaded, for JUN and EGR2, 15 μg of protein was loaded and for analysis of myelin proteins MPZ and MBP, 7.5 μg of protein was loaded. Next, the samples were run in SDS-PAGE and transferred onto a PVDF membrane. After blocking the membrane in 5% skimmed milk (diluted in TBS/0.1% Tween) for 1 h, the membrane was incubated overnight at 4° C. with primary antibodies listed. Experiments were repeated at least three times with fresh samples and representative photographs are shown. Densitometric quantification was done using ImageJ. Measurements were normalized to the loading control Calnexin or Beta Actin.

RNA Extraction and qPCR

RNA was extracted from P60 tibial or optic nerves using Trizol (Invitrogen, 15596026) as previously described (Arthur-Farraj et al., 2017). Nerves from two mice were pooled together for each biological replicate. The integrity and quantity of RNA was determined using Nanodrop (ThermoFisher Scientific) and Agilent 2100 Bioanalyzers (Agilent Technologies). 500 ng of RNA was used per biological replicate for cDNA conversion using QuantiTect reverse transcription kit (Qiagen, 205311). qPCR was run on a BioRad CFX96 using iTaq with SYBR Green (BioRad, 1725124). Ankrd27 and Canx were used as housekeeping genes. Two technical replicates and five biological replicates were run per experiment. Fold change was calculated using the delta CT method. All primers were designed using Primer blast (NCBI) (Ye et al., 2012).

TABLE 1
Primer sequences

| Gene | | SEQ ID | | SEQ ID |
|---|---|---|---|---|
| name | Forward Primer sequence | NO | Reverse Primer sequence | NO |
| Ankrd27 | TCCTGCCAGTTCGAGTCCTA | 3 | AATGACGACAGCCTTTCCATC | 4 |
| | T | | | |
| Canx | CTTCCAGGGGATAAAGGAC | 5 | ACATAGGCACCACCACATTCT | 6 |
| | TTGT | | A | |
| Ccl2 | AGGCTGGAGAGCTACAAGA | 7 | CCCATTCCTTCTTGGGGTCAG | 8 |
| | GG | | | |
| Ccl3 | TGCCCTTGCTGTTCTTCTCT | 9 | GTGGAATCTTCCGGCTGTAG | 10 |
| Ccl4 | AAGCTGCCGGGAGGTGTAA | 11 | TGTCTGCCCTCTCTCTCCTCT | 12 |
| | G | | TG | |
| Ccl5 | TGCCCACGTCAAGGAGTATT | 13 | TCCTAGCTCATCTCCAAATAG | 14 |
| | TC | | TTGATG | |
| Egr2 | CCGTATCCGAGTAGCTTCG | 15 | TCAATGGAGAATTTGCCCATG | 16 |
| | C | | T | |
| Gfap | GGGCGAAGAAAACCGCATC | 17 | TCACCATCCCGCATCTCCA | 18 |
| | A | | | |
| Fos | GGTTTCAACGCCGACTACG | 19 | GTTGGCACTAGAGACGGACA | 20 |
| | A | | G | |
| Il1b | GCCACCTTTTGACAGTGATG | 21 | ATCAGGACAGCCCAGGTCAA | 22 |
| | AGA | | | |
| Il6 | GCCTTCTTGGGACTGATGCT | 23 | GCCATTGCACAACTCTTTTCT | 24 |
| | | | CA | |
| Il10 | GAGAAGCATGGCCCAGAAA | 25 | AAAATCACTCTTCACCTGCTC | 26 |
| | TCAA | | CA | |
| Jun | CCTTCTACGACGATGCCCTC | 27 | GGTTCAAGGTCATGCTCTGTT | 28 |
| | | | T | |
| Mbp | AATCGGCTCACAAGGGATTC | 29 | TCCTCCCAGCTTAAAGATTTT | 30 |
| | A | | GG | |
| Mpz | CGGACAGGGAAATCTATGG | 31 | TGGTAGCGCCAGGTAAAAGA | 32 |
| | TGC | | G | |
| Olig2 | AACCCCGAAAGGTGTGGAT | 33 | TGGCCCCAGGGATGATCTAA | 34 |
| | G | | | |
| Plp1 | CCGCAAAACAGACTAGCCA | 35 | CAAGCCCATGTCTTTGGCAC | 36 |
| | AC | | | |
| Sarm1 | GGACCATGACTGCAAGGAC | 37 | CCGTTGAAGGTGAGTACAGC | 38 |
| | TG | | C | |
| Sox2 | TCGCAGGGAGTTCGCAAAA | 39 | ACCCAGCAAGAACCCTTTCC | 40 |
| | G | | | |
| Sox10 | CTGCTATTCAGGCTCACTAC | 41 | CTCTGTCTTTGGGGTGGTTGG | 42 |
| | AAGAG | | | |
| Xaf1 | GCTGATCTTCCCACAGGAG | 43 | GAGCTAACCTCTGGCACTTCT | 44 |
| | AC | | C | |

### Electron Microscopy

Sciatic and optic nerves were processed as described previously (Gomez-Sanchez et al., 2015) (Gomez-Sanchez et al., 2015). Briefly, samples were fixed in 2.5% glutaraldehyde/2% paraformaldehyde in 0.1 M cacodylate buffer, pH 7.4, overnight at 4° C. Samples were post-fixed with 1% OsO4, embedded in Agar 100 epoxy resin. Transverse ultrathin sections from neonatal (P2) and adult (P60) sciatic nerves (5 mm from the notch) or from adult (P60) optic nerves (2 mm from the chiasm) were taken and mounted on film.

Photographs were taken using a Jeol 1010 electron microscope with a Gatan camera and software. Images were analyzed using ImageJ. Photographs of sciatic nerves were taken at 3000× magnification to measure the number of myelinated axons, non-myelinated axons bigger than 1.5 μm, and Schwann cell nuclei. The nerve area was measured from photographs taken at 200× magnification. Photographs of optic nerves were taken at 12000× magnification to measure the number of myelinated and non-myelinated axons. The nerve area was measured from photographs taken at 200× magnification. N=5 for all experiments (5 biological replicates: 1 nerve per animal, 5 animals, 15-20 sections per nerve).

### Statistical Analysis:

Statistical analyses were performed using Graph-Pad Prism software (version 9.1.2). Results are expressed as mean±SEM. Statistical significance was estimated by Mann-Whitney U-test or unpaired, two tailed students t-test with Bonferonni correction for multiple testing where necessary. P<0.05 was considered statistically significant. N numbers for all experiments are listed in the figure legends. All quantification was blinded to the assessor.

### Results

Sarm1/Sarm1 mRNA Expression and Protein Detection in Vertebrate Schwann Cells, Satellite Glia and Oligodendrocytes.

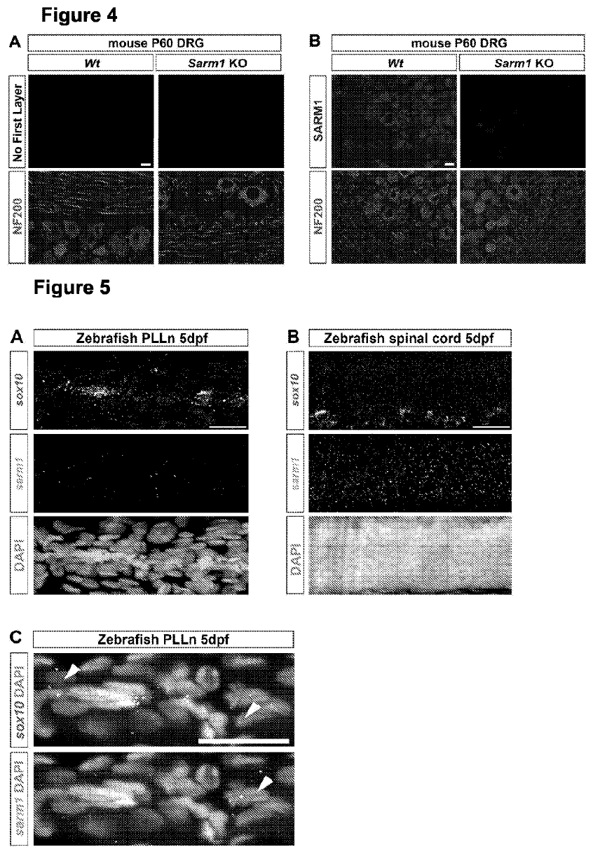

In order to investigate the presence of SARM1 protein in various cell types in the murine nervous system a validated polyclonal antibody generated by C.-Y. Chen et al., 2011 was used in transverse cryosections of adult (P60) tibial and optic nerve. As expected, it was found that SARM1 expression colocalised with the axonal markers neurofilament and beta-III tubulin in sciatic and optic nerve respectively (FIGS. 3A and B). Additionally, it was found that SARM1 protein was present ubiquitously, at high levels, in large and small dorsal root ganglion (DRG) neuron cell bodies, in vivo, compared to lower levels within their axons (FIG. 3C). Importantly, we saw no staining in control sections without primary antibody nor in Sarm1 knockout (KO) DRG neurons (FIGS. 4A and B). To test if there were detectable levels of SARM1 protein in myelinating and non-myelinating Schwann cells, satellite glia and oligodendrocytes SARM1 and SOX10 immunohistochemistry in cryosections from tibial nerves was performed, DRGs and optic nerves and in PNS tissue found no colocalization in multiple sections from 3 separate non-littermate animals (FIG. 3D-E). However, in optic nerve sections SARM1 protein appeared present in a perinuclear staining pattern around SOX10 positive nuclei (FIG. 3F). Thus, SARM1 protein appears restricted to neuronal populations in the PNS and is not detectable in adult murine Schwann cells and satellite glia. However in the CNS, SARM1 protein is potentially present in oligodendrocytes.

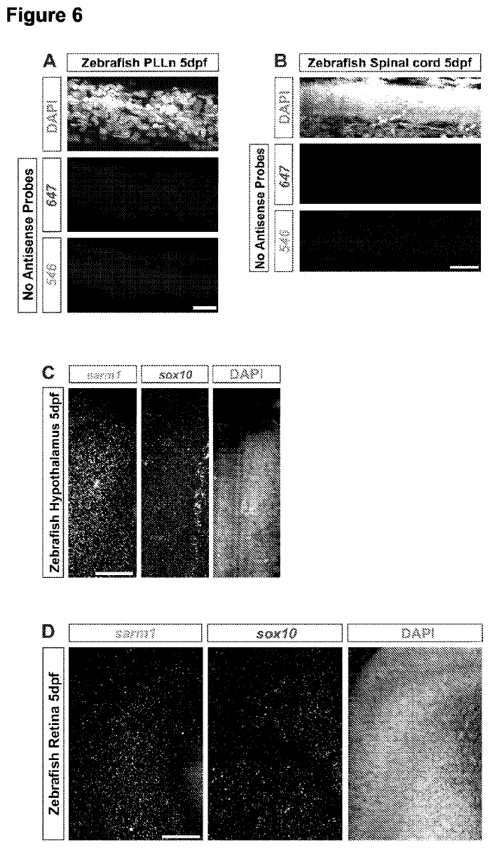

To identify if sarm1 mRNA is expressed in myelinating glia third generation in situ hybridisation chain reaction (HCR) was used, in five-day post fertilisation (5dpf) zebrafish larvae, where both oligodendrocytes and Schwann cells, in the CNS and PNS, respectively, are initiating myelination (D'Rozario et al., 2017; Choi et al., 2018). HCR probes targeted to sarm1 and sox 10 were used to label cells of the oligodendrocyte and Schwann cell lineage. In the posterior lateral line nerve (PLLn) strong expression of sox10 but little sarm1 expression was seen, though importantly no sarm1 signal was seen when antisense probes were omitted (FIG. 5A; FIGS. 6A and B). In the spinal cord, while sox 10 expression was seen mainly concentrated in the ventral and dorsal spinal cord tracts, sarm1 expression was visualized more diffusely throughout the whole spinal cord though without much overlap with the sox10 signal (FIG. 5B). Some overlap of sox10 and sarm1 is expected as sox10 is also expressed in a subset of spinal interneurons (Almeida and Lyons, 2015). Interestingly, at two other regions of the nervous system (hypothalamus and retina), it was found that there was no substantial overlap of sox 10 and sarm1 expression (FIGS. 6C and D). In order to investigate whether sarm1 and sox 10 were co-expressed in the same cells in the PNS and CNS, individual confocal slices (616 nm optical thickness) were examined and it was found that only co-expression of both markers in nuclei along the PLLn were seen infrequently. Specifically, in the PLLn we found that 22.4%+/−3.161 (n=5 larvae, 5 sections per larvae) of Schwann cells express a low level of sarm1 (FIG. 5C).

In combination, these results are consistent with the view that Schwann cells and oligodendrocytes do express low levels of Sarm1 mRNA but only oligodendrocytes potentially have detectable levels of SARM1 protein.

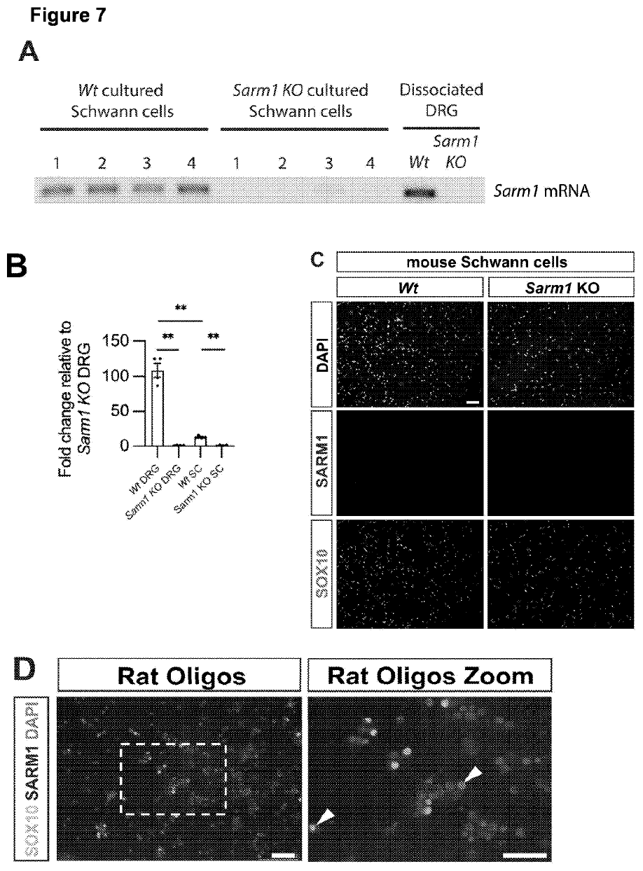

Cultured oligodendrocytes, but not Schwann cells, have detectable SARM1 protein and are vulnerable to specific, SARM1 activator-induced cell death. Given the observed expression of sarm1 mRNA in Schwann cells and oligodendrocytes in zebrafish, it was decided to test whether mouse Schwann cells also express Sarm1 mRNA. Although Sarm1 mRNA in cultured mouse Schwann cells was detected and not in Sarm1 KO Schwann cells, this was at substantially lower levels than that of cultured mouse DRG neurons (FIGS. 7A and B).

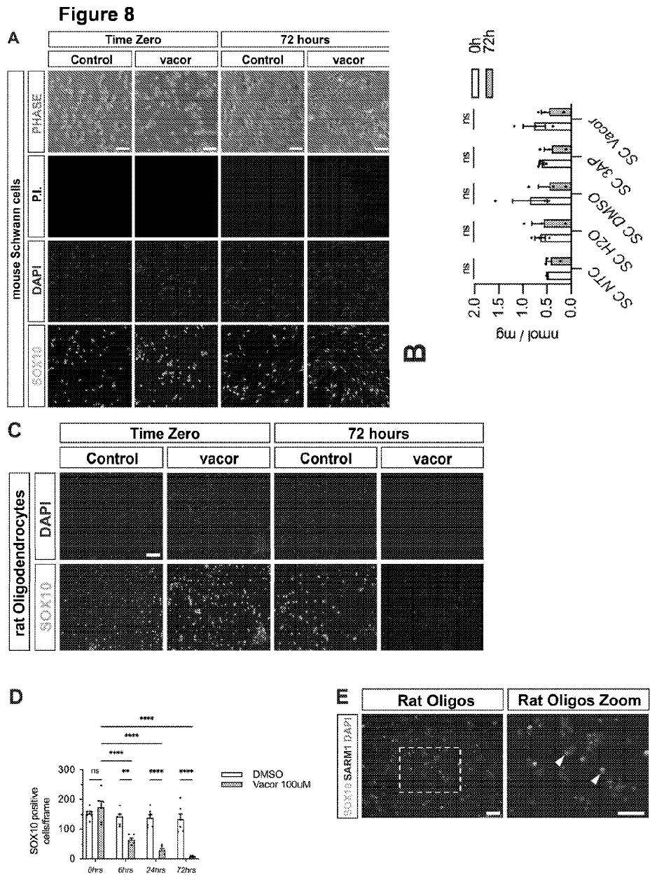

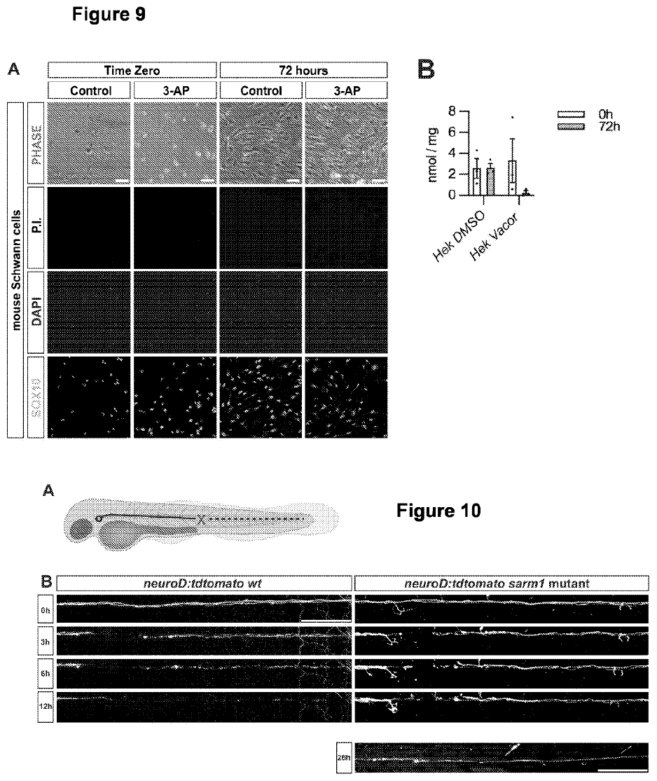

Since it was not possible to identify SARM1 protein in Schwann cells but did potentially detect SARM1 in cells of the oligodendroglia lineage in vivo, it was decided to further explore whether these cells may contain small amounts of functional SARM1 protein. In order to test this hypothesis, cultures of freshly isolated mouse Schwann cells and rat oligodendrocytes were treated with two separate SARM1 activators, vacor and 3-AP. Addition of high dose vacor or 3-AP to neuronal cultures induces rapid SARM1 activation, profound Nicotinamide adenine dinucleotide (NAD⁺) depletion and axonal degeneration and cell death within hours (Loreto et al., 2021; Wu et al., 2021). It was found that mouse Schwann cells were completely insensitive to both vacor and 3-AP treatment at high doses for long periods of time (up to 72 hours) ((FIG. 8A and FIG. 9A). In further support of this, Schwann cells treated with vacor or 3-AP for 72 hours showed no reduction in intracellular NAD⁺ levels, unlike in Human embryonic kidney cells, which express low levels of SARM1 protein (FIG. 8B and FIG. 9B) On the contrary it was found that oligodendrocytes were sensitive to vacor addition and died within hours, judged by propidium iodide staining and SOX10 and DAPI nuclear labelling (FIGS. 8C and D). Oligodendrocyte cultures were then stained and the presence of detectable levels of SARM1 was found using two different antibodies in contrast to mouse Schwann cells (FIG. 8E and FIGS. 7C and D). Thus, cultured oligodendrocytes, in contrast to Schwann cells, appear to contain sufficient endogenous SARM1 protein to induce cell death.

### Initiation of CNS and PNS Myelination Proceeds Normally in Sarm1 Mutant Zebrafish Larvae

Since inhibition of SARM1 function is being proposed to treat neurological disease, it is crucial to identify whether loss of SARM1, cell or non-cell autonomously, leads to any disruption of vertebrate CNS or PNS myelination. This question is particularly pertinent given that functional SARM1 protein in oligodendrocytes has been identified. To date, no studies have performed an in-depth quantitative analysis of CNS and PNS myelination in the absence of SARM1 function. To test whether Sarm1 is required for correct initiation of myelination in zebrafish myelination and myelin gene expression in the spinal cord and PLLn in wild-type (wt) and sarm1^{SA11193/SA11193} (mutant) larvae at 5dpf was assessed. The sarm1^{SA11193} mutants fish harbour a point mutation (C>A) in exon 2, which introduces a premature stop codon (Busch-Nentwich et al., 2013). sarm1^{SA11193} mutants are viable, fertile and appear morphologically indistinct from wt fish (data not shown). To confirm that sarm1 mutants behave functionally like sarm1 null animals, which have substantially delayed axonal degeneration after traumatic injury, wt and homozygous mutant fish were injected at the one-celled zygote stage with a neuroD:tdTomato DNA construct. 2-photon laser axotomy of tdTomato labelled PLLn neurons at 4dpf was performed and the axons distal to the injury site were live imaged (FIG. 10A). It was found that while wt axons started to degenerate between 2 hr 40 minutes and 3 hour 20 minutes (n=7), axons in sarm1 mutant fish remained intact even 26 hours after axotomy, which was the limit of our imaging abilities due to UK home office regulations (FIG. 10B). This confirmed that sarm1^{SA11193} mutant zebrafish phenotypically behave like Sarm1 null mice and are similar to a Crispr-Cas9 generated sarm1 null mutant zebrafish (Tian et al., 2020).

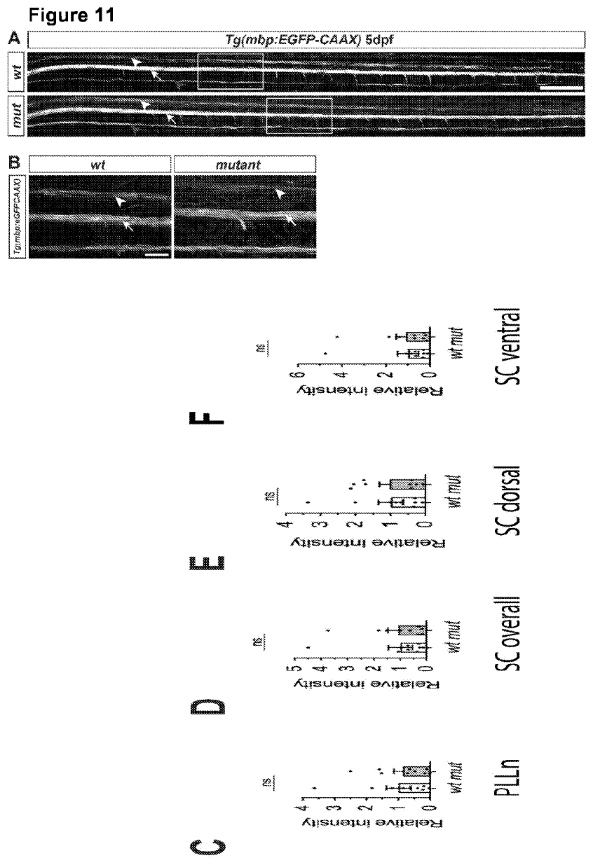

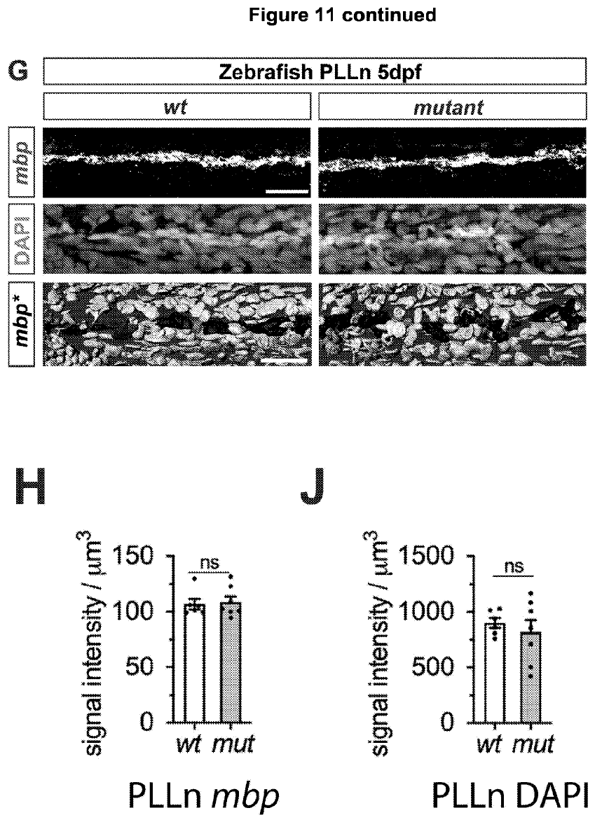

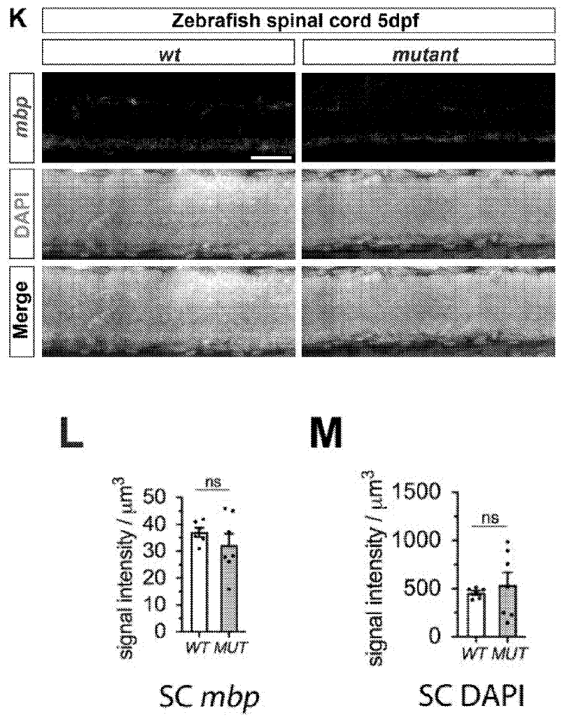

To assess PNS and CNS myelination, sarm1^{SA11193} mutants were crossed with Tg (mbp: EGFP-CAAX) zebrafish that express membrane bound GFP under the myelin basic protein (mbp) promoter (Almeida et al., 2011). Normal formation of long GFP-labelled myelin segments in the dorsal and ventral spinal cord was observed as well as PLLn in both 5dpf sarm1^{SA11193} mutants and wt fish and quantification showed no differences in PLLn and spinal cord myelination (FIG. 11A-F).

In order to quantify myelin gene expression between wt and sarm1 mutant zebrafish, mbp HCR was performed. It was found that there were equivalent levels of mbp expression in the spinal cord and in the PLLn in wt and sarm1^{SA11193} mutant fish and importantly, there were no differences in cell numbers in either structure between genotypes (FIG. 11G-M).

Collectively these experiments demonstrate that myelination proceeds normally in both the PLLn and spinal cord in zebrafish larvae in the absence of Sarm1 function.

### PNS Myelination and Myelin Maintenance are Normal in the Sarm1 KO Mouse

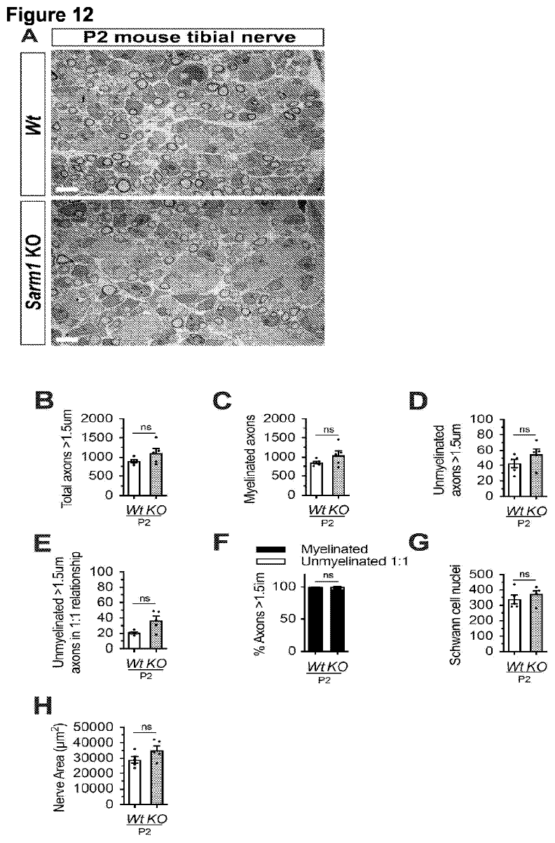

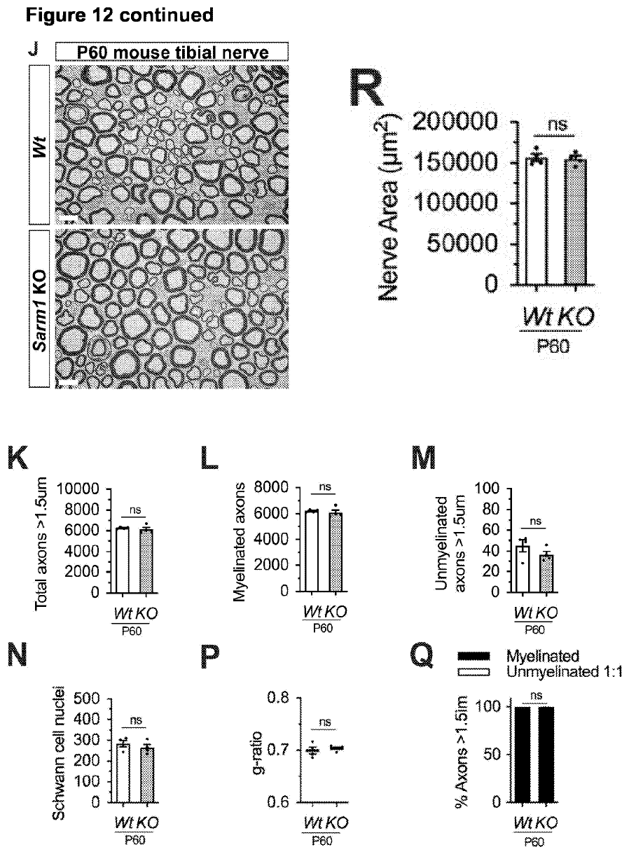

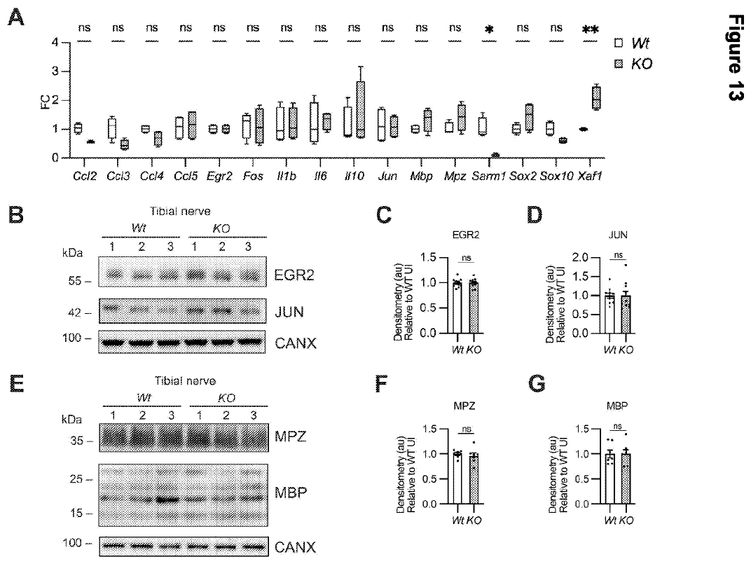

Given that zebrafish embryos appear to myelinate normally without functional Sarm1, it was next tested whether PNS myelination in Sarm1 KO mice is initiated correctly. PNS myelination starts shortly after birth in murine peripheral nerves (Jessen and Mirsky, 2005). Postnatal (P) day 2 transverse sections were looked at through the tibial nerve by transmission electron microscopy (EM) and it was noted that while there was a non-significant trend towards slightly more axons and Schwann cells in the Sarm1 KO nerves compared to Wt (n=5), there was no difference in the proportion of large calibre axons that were myelinated (FIG. 12A-H).

Adult (P60) tibial nerves were then looked at by EM to investigate whether myelin maintenance is affected by loss of Sarm1 in the PNS. Interestingly a trend towards increased axon numbers in the Sarm1 KO at P60 (FIG. 12J-M) was no longer seen. Furthermore, the proportion of unmyelinated and myelinated axons, Schwann cell nuclei as well as myelin sheath thickness, measured by g-ratio and nerve area were all similar between Wt and Sarm1 KO samples (FIG. 12N-R).

To test whether there were any gene expression differences between Wt and Sarm1 KO tibial nerves gene and protein expression was looked at by quantitative reverse transcriptase polymerase chain reaction (qPCR) and western blot respectively. Sarm1 KO nerves had no differences in myelin gene or other Schwann cell-specific gene expression (Cdh1,Egr2,Mbp,Mpz, Sox10), immature or Schwann cell injury gene expression (Fos, Jun, Sox2) or cytokine or chemokine expression (Ccl2, Ccl3, Ccl4, Ccl5, Il1b, IL6, IL10). It was however detected that Sarm1 KO tibial nerves expressed a two-fold higher level of the pro-apoptotic gene, Xaf1, which has also been reported to be upregulated in mouse brain and macrophages from this particular Sarm1 KO mouse line (FIG. 13A; Uccellini et al., 2020; Zhu et al., 2019). Additionally, it was found that there was no differences in protein levels for EGR2/KROX-20, JUN, and myelin proteins, MPZ and MBP (FIG. 13B-G).

Thus, myelination and myelin maintenance are unperturbed and myelin gene and protein expression are normal in the PNS in the absence of Sarm1 in the mouse.

### CNS Myelination and Myelin Gene Expression are Normal in the Sarm1 KO Mouse

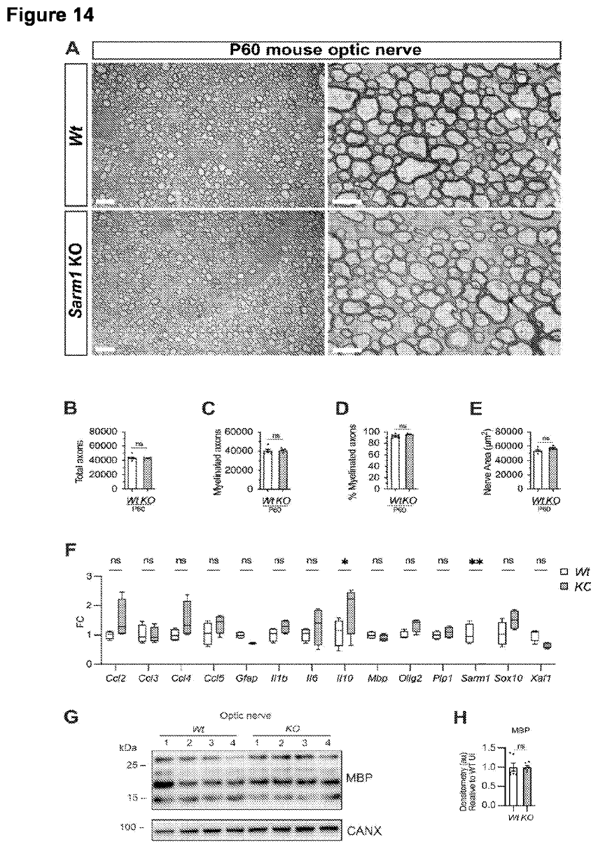

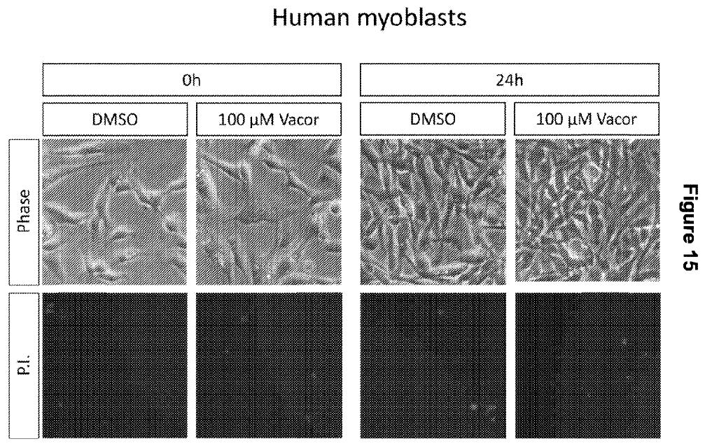

It was shown that oligodendrocytes in the dorsal and ventral spinal cord of Sarm1 mutant zebrafish larvae myelinate normally. To confirm whether CNS myelination is also unaffected in Sarm1 KO mice, adult P60 optic nerves were assessed by EM. It was found that Sarm1 KO optic nerves were morphologically indistinct from Wt samples, with similar numbers of total axons, ratio of myelinated to unmyelinated axons and a similar cross-sectional nerve area (FIG. 14A-F). Furthermore, by qPCR, Sarm1 KO optic nerves showed no differences in gene expression for oligodendrocyte/myelin markers (Mbp, Olig2, Plp1, Sox10), cytokine or chemokine expression (Ccl2, Ccl3, Ccl4, Ccl5, IL1b, IL6), apart from an upregulation of IL10 (FIG. 14G). Interestingly although there was a significant difference in Sarm1 mRNA expression between WT and KO samples, as one would expect, any upregulation of Xaf1 in the optic nerve was nt detected (FIG. 14G). MBP protein levels were also tested by western blot in Sarm1 KO optic nerves and it was found that there was no differences compared to WT nerves (FIG. 14H).

In conclusion there is no observable defects in myelin in adult Sarm 1 KO mouse optic nerves.

### Discussion

A number of studies investigating the role of SARM1 in the nervous system have documented that PNS and CNS myelin appears normal in the adult Sarm1 KO mouse however there have been no in-depth quantitative studies of both PNS and CNS myelination to confirm or refute this (Osterloh et al., 2012; Geisler et al., 2016; Marion et al., 2019; Ko et al., 2020). Additionally, there has been one study that generated a Crispr-Cas9 sarm1 mutant zebrafish however they did not comment on whether PNS or CNS myelination was normal (Tian et al., 2020). Furthermore, there have been no studies investigating whether SARM1 is present in PNS and CNS myelinating glia.

It is shown herein that sarm1 mRNA is expressed at low levels in Schwann cells and oligodendrocytes in developing zebrafish larvae and that cultured mouse Schwann cells also express Sarm1 mRNA. However, SARM1 protein was detected in in oligodendrocytes but not in Schwann cells in the mature mouse nervous system. It is also shown that in the absence of Sarm1/SARM1, PNS and CNS myelination proceeds normally in both zebrafish and mice and that SARM1 does not play a role in myelin maintenance in the murine PNS or CNS. This adds to previous findings that conduction velocities measured by neurophysiological investigation of adult Sarm1 KO mouse sciatic nerves were similar to control mice and that Sarm1 KO adult mice had normal myelinated axon numbers in the corpus callosum (Geisler et al., 2016; Marion et al., 2019).

SARM1, unlike other members of the MyD88 family, is abundant in the murine nervous system, particularly in neurons (Kim et al., 2007; Chen et al., 2011). A previous study documented that SARM1 protein is not found in mouse microglia (Lin et al., 2014), and it is shown herein that it is likely present, in vivo, in oligodendrocytes but not in myelinating and non-myelinating Schwann cells. Interestingly, a recent study has shown that SARM1 protein is detectable in mouse astrocytes and that it appears to play a role in neuroinflammation (Liu et al., 2021). In the PNS, in addition to Schwann cells, it is shown herein that satellite glia, do not appear to have identifiable levels of SARM1 protein. SARM1 is also likely not to be present at high levels in PNS resident macrophages since studies on other peripheral macrophage populations found relatively low levels of SARM1 in these cells and no effect of Sarm1 deletion on macrophage function or gene expression (Kim et al., 2007; Uccellini et al., 2020).

It has been postulated that one of the purposes of SARM1, a toll like adapter protein and remnant of the innate immune system, and the wider axon degeneration machinery is to bring about compartmentalized neurodegeneration to prevent spread of viral pathogens throughout the nervous system (Tsunoda, 2008). In mouse CNS myelinating cocultures, loss of Sarm1 protects neuronal somas against infection and cell death (Crawford et al., 2022). Zika virus preferentially infects oligodendroglia and astroglia in neuron/glia cocultures and causes glia cell death (Cumberworth et al., 2017). Activation of SARM1 is known to cause neuronal cell body death independently of axonal degeneration (Sasaki et al., 2020; Loreto et al., 2021). It is currently unknown if CNS glial cells upregulate Sarm1 expression in response to zika infection and whether the glial cell death, is SARM1 dependent. Interestingly, astrocytes do appear to upregulate expression of SARM1 in response to spinal cord injury (Liu et al., 2021).

In summary, the findings herein suggest that using Sarm1 mutant mice and sarm1 mutant zebrafish to model axonopathy in various disease models is a viable approach as myelination is unlikely to be perturbed by Sarm1 deletion.

### Example 2

### Materials and Methods

### Human Primary Myoblasts and Human Dermal Skin Fibroblasts Culture, Treatment and Cell Death Assays

Human primary myoblasts and human dermal skin fibroblasts were cultured following previously described protocols (Kisiel and Klar 2019; Rozwadowska et al. 2022). Drug treatment was started when cells reached the desired confluency. To visualise cell death, 1 μg/ml propidium iodide (PI) (Thermo Fisher Scientific) was added to the media at the same time of drug addition (Loreto et al. 2021). Phase contrast and fluorescence microscopy images were acquired on a DMi8 upright fluorescence microscope (Leica microsystems) coupled to a monochrome digital camera (Hamamatsu C4742-95). The objective used was HCXPL 20 X/0.40 CORR.

### Axon Degeneration and Regeneration Assays in Human DRGs

Human foetal DRG ganglia were dissected, dissociated and plated in microfluidic chambers (150 μm barrier, XONA Microfluidics) in 35 mm tissue culture dishes pre-coated with poly-L-lysine (100 μg/ml for 1 hr; Merck) and laminin (20 μg/ml for 1 hr; Merck) in Dulbecco's Modified Eagle's Medium (DMEM, Gibco) with 1% penicillin/streptomycin, 33 ng/ml 2.5 S NGF (all Invitrogen) and 2% B27 (Gibco). 4 μM aphidicolin (Merck) was used to reduce proliferation and viability of small numbers of non-neuronal cells. On DIV7, a difference of 100 μl of media between chambers was introduced and drugs were added to the compartment with the lower hydrostatic pressure. Phase contrast microscopy images were acquired on a DMi8 upright fluorescence microscope (Leica microsystems) coupled to a monochrome digital camera (Hamamatsu C4742-95). The objective used was HCXPL 20 X/0.40 CORR.

### Results

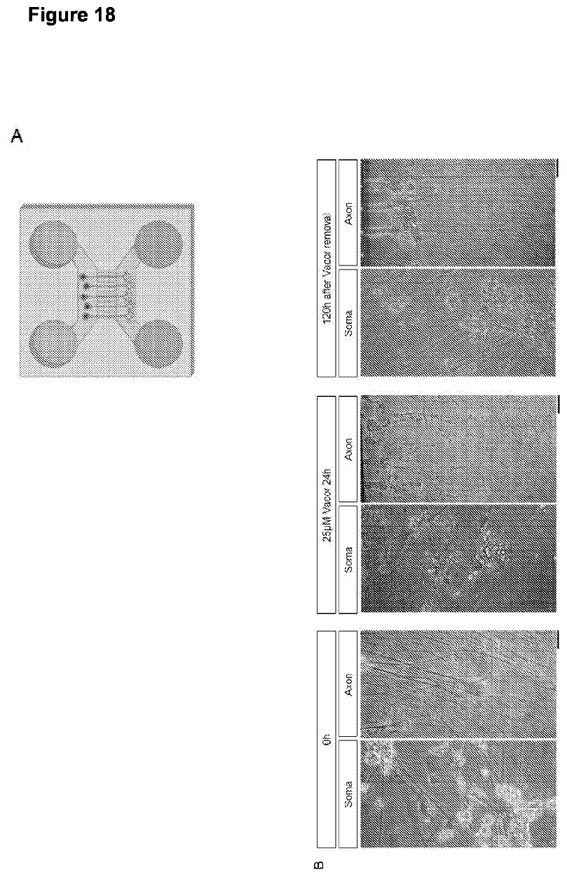

To test if human muscle cells and skin cells express any functional SARM1 protein, human primary myoblasts (muscle cells) and human dermal skin fibroblasts (skin cells) cultures were treated with the SARM1 agent vacor. It was found that human myoblasts and human dermal skin fibroblasts were completely insensitive to induction of cell death by high dose vacor (100 μM) as evidenced by propidium iodide staining (FIG. 15,17). These findings indicate that SARM1 protein is not present or present at functionally negligible levels in human myoblasts (muscle cells) and human dermal skin fibroblasts (skin cells). Next it was tested if human axons can regenerate following localised axon degeneration caused by the SARM1 agent vacor. To this end, human foetal DRG axons were cultured in microfluidic chambers to allow localised treatment of axons (FIG. 18). DRG axons degenerated 24 h after addition of 25 μM Vacor in the axonal compartment, while untreated neuronal cell bodies remained healthy. Vacor was then removed from the axonal compartment 24 h after treatment, when axons have already degenerated, and fresh media was added to the axonal compartment. Following the addition of fresh media, DRG axons started to regrow and fully repopulated the axonal compartment withing 120 h after Vacor removal. This demonstrates that human neurons can regenerate their axons following degeneration caused by localised administration of a SARM1 agent.

### Discussion

For SARM1 agents to have a temporary, reversible and selective effect, it is important that these agents cause selective nerve terminal degeneration in the area where they are administered in cells expressing SARM1, such as neurons, and not on surrounding cells, that do not express or express functionally negligible levels of SARM1, in the site of application, such as skin cells, muscle cells and Schwann cells. It is also important that neurons can regenerate their axons to avoid permanent disability after treatment with SARM1 agents. Firstly, it is previously unknown whether SARM1 is present at functional levels in non-neuronal cells of the PNS and cells of target organs (i.e. skin and muscle cells) that the PNS innervates. As discussed in example 3 and from the data provided herein, it is important to note that it cannot simply be assumed from previous studies and publications that SARM1 is selectively present in neurons. Here it is shown for the first time that human myoblasts (muscle cells) and human dermal skin fibroblasts (skin cells) are insensitive to the SARM1 agent vacor. These findings add further support to the proposal to use SARM1 agents as a cell selective method of peripheral neuroablation to treat conditions such as dystonia, spasticity, peripheral neuropathic pain, autonomic dysfunction and for application in cosmetic conditions.

In addition, it is shown that human DRG axons degenerate after local administration of the SARM1 agent vacor, even at lower doses compared to what was used on other cell types such as muscle cells, skin cells and Schwann cells. This is the first time that it has been shown that axons can regenerate after degeneration caused by the localised administration of a SARM1 agent such as vacor. These findings, in addition to findings that Schwann cells are insensitive to SARM1 agents such as vacor, add further support to the proposal to use SARM1 agents as a temporary, reversible and cell selective method of peripheral neuroablation, allowing for regeneration of axons and terminal aborizations, to treat conditions such as dystonia, spasticity, peripheral neuropathic pain, autonomic dysfunction and for application in cosmetic conditions. It is important to know that these experiments were performed on human primary cells, further supporting the feasibility of the proposed use of SARM1 agents.

### Example 3

### Materials and Methods

### Sciatic Nerve Transection Surgery

All research complied with the Animals (Scienti c Procedures) Act 1986 and the University of Cambridge Animal Welfare and Ethical Review Body (AWERB) and the European Union guidelines and protocols were approved by the Comit e de Bio etica y Bioseguridad del Instituto de Neurociencias de Alicante, Universidad Miguel Hern andez de Elche and Consejo Superior de Investigaciones Cient cas (http://in.umh-csic.es/). Wildtype (Wt) C57BL/6J mice were obtained from Charles River Laboratories or maintained as a Wildtype colony. Surgeries were performed on 6-8 week old Wildtype or Sarm1 knockout mice. Mice were anesthetised with 2% isourane, sciatic nerves were exposed and cuts performed at the sciatic notch (Arthur-Farraj et al., 2012). The wound was closed using veterinary autoclips (AutoClip System). Nerves were harvested after 3, 6, 9, 12, 18, or 24 hours, or after 2, 5, 7, or 14 days, and the contralateral uninjured nerve was used as a control.

### Mouse Schwann Cell Expansion

Mouse SCs were passaged on PLL/laminin coated dishes in the presence of low serum (0.5% horse serum), 10 ng/ml Bneuregulin1 (BNRG1), and a low concentration cyclic adenosine monophosphate (CAMP) elevating signal, such as 2 μM forskolin or 100 μM dibutryl cAMP (Arthur-Farraj et al., 2011). Full protocol is described in (Mutschler et al., 2023).

All other relevant methods are described in Example 1.

### Results

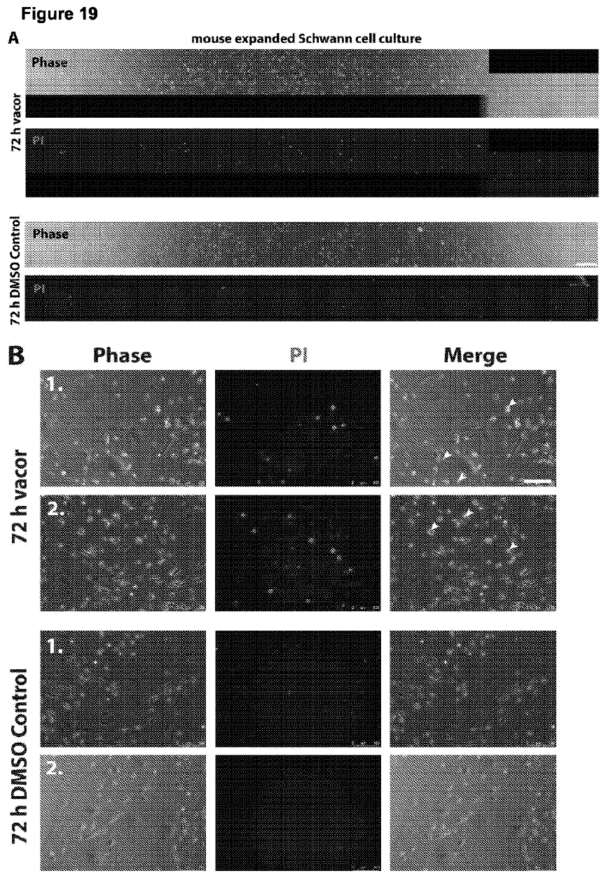

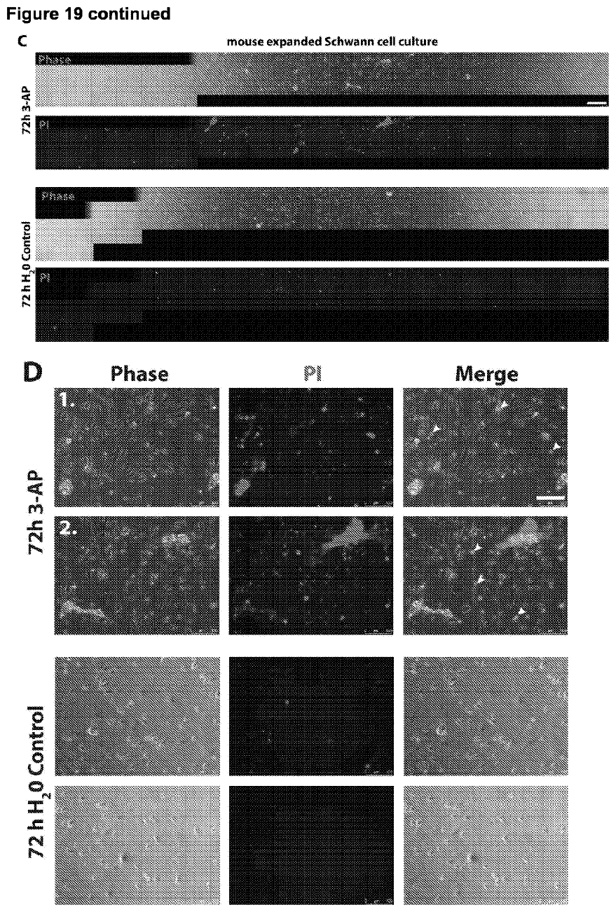

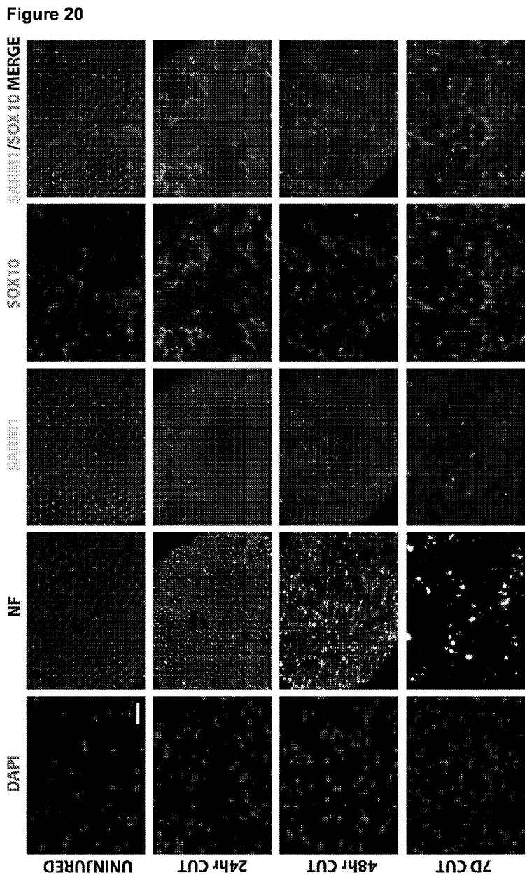

In Example 1 freshly isolated Schwann cells from neonatal mouse sciatic nerves were used to test sensitivity of Schwann cells to the SARM1 agents, vacor and 3-AP. It was found that Schwann cells were completely insensitive to SARM1 agonists (FIGS. 7, 8 and 9). These findings are consistent with the discovery that SARM1 protein is not present in mouse Schwann cells in uninjured sciatic nerves and sarm1 mRNA is very lowly expressed in only a subset of Schwann cells in the zebrafish PLLn (FIGS. 3 and 5). Early on in investigations experiments using expanded mouse Schwann cells that had been passaged three times in cell culture over a 2-week period after extraction from mouse nerves were also performed. When expanded mouse Schwann cells were treated with the same concentrations of vacor or 3-AP used in Example 1 it was found that expanded mouse Schwann cells were susceptible to cellular dysfunction and death as evidenced by propidium iodide staining (FIG. 19). Given that cultured Schwann cells can replicate hallmarks of in vivo injured Schwann cells, or repair Schwann cells, it was wanted to test whether Schwann cells contain functional SARM1 protein after nerve injury (Arthur-Farraj et al., 2017). Sections of injured mouse tibial nerve were immunostained at various time points after nerve injury (24 and 48 hours and 7 days). No colocalization of SARM1 protein with SOX10, a marker of Schwann cells, was seen and instead colocalization of SARM1 with the axonal protein neurofilament light chain was seen (FIG. 20). These findings demonstrate that Schwann cells do not contain detectable SARM1 protein at various timepoints after nerve injury. However, after prolonged passaging, cultured Schwann cells do become sensitive to SARM1 agents.

### Discussion

When axons degenerate, Schwann cells become injury activated and transform into repair Schwann cells (Arthur-Farraj and Coleman, 2021). This injury induced Schwann cell transformation will occur when SARM1 agents are applied to peripheral neural structures and target organs (e.g. skin and muscle) and thus it was important to test if Schwann cells change their expression profile for SARM1 in an injury situation. The finding that Schwann cells at various time points after nerve injury do not contain detectable levels of SARM1 protein in vivo is consistent with findings that SARM1 is absent from Schwann cells in uninjured nerves and in freshly isolated Schwann cells in culture. This, finding adds further support to the proposal to use SARM1 agents as a cell selective method of peripheral neuroablation to treat conditions such as dystonia, spasticity, peripheral neuropathic pain, autonomic dysfunction and for application in cosmetic conditions. The fact that it was found that prolonged passaging of cultured mouse Schwann cells lead to them becoming sensitive to vacor and 3-AP induced cell death was surprising and inconsistent with all other observations. However, prolonged passaging in cell culture is very artificial and it has been shown that Schwann cells can lose hallmarks of their phenotype and change expression of key markers, such as downregulation of JUN (Wagstaff et al., 2021). Additionally prolonged passaging of cells leads to development of multiple somatic mutations (Rouhani et al., 2022). This finding likely represents an artifact of prolonged cell culture, and this is exemplified by the observation that the freshly isolated Schwann cells that have been in culture for only 24 hours are insensitive to SARM1 agents. It is important to note that this result does highlight that it cannot simply be assumed from prior studies that SARM1 is selectively present in neurons and not other cell types as surprisingly shown herein that firstly, CNS myelinating glia contain functional SARM1 and that secondly, Schwann cells do not contain functional SARM1 but can become sensitive to SARM1 agents in artificial contexts, such as prolonged cell culture. Importantly, the vast majority of in vitro Schwann cell experiments in the field are carried out on Schwann cells that have been passaged several times. Thus the insensitivity of Schwann cells to SARM1 agents was only uncovered when an unconventional approach to use short term culture of Schwann cells to maintain them in a closer physiologic state to in vivo was performed.

### REFERENCES

Arthur-Farraj, P. and Coleman, M. P. (2021). Correction to: Lessons from Injury: How Nerve Injury Studies Reveal Basic Biological Mechanisms and Therapeutic Opportunities for Peripheral Nerve Diseases. Neurotherapeutics 18, 2757.
Arthur-Farraj, P., Wanek, K., Hantke, J., Davis, C. M., Jayakar, A., Parkinson, D. B., Mirsky, R. and Jessen, K. R. (2011). Mouse schwann cells need both NRG1 and cyclic AMP to myelinate. Glia 59, 720-733.
Arthur-Farraj, P. J., Latouche, M., Wilton, D. K., Quintes, S., Chabrol, E., Banerjee, A., Woodhoo, A., Jenkins, B., Rahman, M., Turmaine, M., et al. (2012). c-Jun reprograms Schwann cells of injured nerves to generate a repair cell essential for regeneration. Neuron 75, 633-647.
Arthur-Farraj, P. J., Morgan, C. C., Adamowicz, M., Gomez-Sanchez, J. A., Fazal, S. V., Beucher, A., Razzaghi, B., Mirsky, R., Jessen, K. R. and Aitman, T. J. (2017). Changes in the Coding and Non-coding Transcriptome and DNA Methylome that Define the Schwann Cell Repair Phenotype after Nerve Injury. CellReports 20, 2719-2734.
Mutschler, C., Fazal, S. V., Schumacher, N., Loreto, A., Coleman, M. P. and Arthur-Farraj, P. (2023). Schwann cells are axo-protective after injury irrespective of myelination status in mouse Schwann cell-neuron cocultures. J. Cell Sci. 136.
Rouhani, F. J., Zou, X., Danecek, P., Badja, C., Amarante, T. D., Koh, G., Wu, Q., Memari, Y., Durbin, R., Martincorena, I., et al. (2022). Substantial somatic genomic variation and selection for BCOR mutations in human induced pluripotent stem cells. Nat. Genet. 54, 1406-1416.
Wagstaff, L. J., Gomez-Sanchez, J. A., Fazal, S. V., Otto, G. W., Kilpatrick, A. M., Michael, K., Wong, L. Y. N., Ma, K. H., Turmaine, M., Svaren, J., et al. (2021). Failures of nerve regeneration caused by aging or chronic denervation are rescued by restoring Schwann cell c-Jun. Elife 10.
Kisiel M A, Klar A S (2019) Isolation and Culture of Human Dermal Fibroblasts. In: Böttcher-Haberzeth S, Biedermann T (eds) Skin Tissue Engineering: Methods and Protocols. Springer, New York, N Y, pp 71-78
Loreto A, Angeletti C, Gu W, et al (2021) Neurotoxin-mediated—potent activation of the axon degeneration regulator SARM1. eLife 10:e72823. https://doi.org/10.7554/eLife.72823
Rozwadowska N, Sikorska M, Bozyk K, et al (2022) Optimization of human myoblasts culture under different media conditions for application in the in vitro studies. American Journal of Stem Cells 11:1
Almeida R G, Czopka T, Ffrench-Constant C, Lyons D A. 2011. Individual axons regulate the myelinating potential of single oligodendrocytes in vivo. Dev Camb Engl 138:4443-4450.
Arthur-Farraj P, Wanek K, Hantke J, Davis C M, Jayakar A, Parkinson D B, Mirsky R, Jessen K R. 2011. Mouse schwann cells need both NRG1 and cyclic AMP to myelinate. Glia 59:720-733.
Arthur-Farraj P J, Morgan C C, Adamowicz M, Gomez-Sanchez J A, Fazal S V, Beucher A, Razzaghi B, Mirsky R, Jessen K R, Aitman T J. 2017. Changes in the Coding and Non-coding Transcriptome and DNA Methylome that Define the Schwann Cell Repair Phenotype after Nerve Injury. Cell Rep 20:2719-2734.
Bloom A J, Mao X, Strickland A, Sasaki Y, Milbrandt J, DiAntonio A. 2022. Constitutively active SARM1 variants that induce neuropathy are enriched in ALS patients. Mol Neurodegener 17:1.
Bosanac T, Hughes R O, Engber T, Devraj R, Brearley A, Danker K, Young K, Kopatz J, Hermann M, Berthemy A, Boyce S, Bentley J, Krauss R. 2021. Pharmacological SARM1 inhibition protects axon structure and function in paclitaxel-induced peripheral neuropathy. Brain [Internet]. Available from: https://doi.org/10.1093/brain/awab184
Busch-Nentwich et al., 2013. Available from: https://zfin.org/ZDB-PUB-130425-4
Chen C-Y, Lin C-W, Chang C-Y, Jiang S-T, Hsueh Y-P. 2011. Sarm1, a negative regulator of innate immunity, interacts with syndecan-2 and regulates neuronal morphology. J Cell Biol 193:769-784.
Chen Y-H, Sasaki Y, DiAntonio A, Milbrandt J. 2021. SARM1 is required in human derived sensory neurons for injury-induced and neurotoxic axon degeneration. Exp Neurol: 113636.
Cheng Y, Liu J, Luan Y, Liu Z, Lai H, Zhong W, Yang Y, Yu H, Feng N, Wang H, Huang R, He Z, Yan M, Zhang F, Sun Y-G, Ying H, Guo F, Zhai Q. 2019. Sarm1 Gene Deficiency Attenuates Diabetic Peripheral Neuropathy in Mice. Diabetes 68:2120-2130.
Choi HMT, Schwarzkopf M, Fornace M E, Acharya A, Artavanis G, Stegmaier J, Cunha A, Pierce N A. 2018. Third-generation in situ hybridization chain reaction: multiplexed, quantitative, sensitive, versatile, robust. Dev Camb Engl 145: dev165753.
Coleman M P, Hoke A. 2020. Programmed axon degeneration: from mouse to mechanism to medicine. Nat Rev Neurosci 21:183-196.
Crawford C L, Antoniou C, Komarek L, Schultz V, Donald C L, Montague P, Barnett S C, Linington C, Willison H J, Kohl A, Coleman M P, Edgar J M. 2022. SARM1 Depletion Slows Axon Degeneration in a CNS Model of Neurotropic Viral Infection. Front Mol Neurosci [Internet] 15. Available from: https://www.frontiersin.org/article/10.3389/fnmol.2022.860410
Cumberworth S L, Barrie J A, Cunningham M E, de Figueiredo DPG, Schultz V, Wilder-Smith A J, Brennan B, Pena L J, Freitas de Oliveira França R, Linington C, Barnett S C, Willison H J, Kohl A, Edgar J M. 2017. Zika virus tropism and interactions in myelinating neural cell cultures: CNS cells and myelin are preferentially affected. Acta Neuropathol Commun 5:50.
D'Rozario M, Monk K R, Petersen S C. 2017. Analysis of myelinated axon formation in zebrafish. Methods Cell Biol 138:383-414.
Fogh I, Ratti A, Gellera C, Lin K, Tiloca C, Moskvina V, Corrado L, Sorarù G, Cereda C, Corti S, SLAGEN Consortium and Collaborators. 2014. A genome-wide association meta-analysis identifies a novel locus at 17q11.2 associated with sporadic amyotrophic lateral sclerosis. Hum Mol Genet 23:2220-2231.
Geisler S, Doan R A, Cheng G C, Cetinkaya-Fisgin A, Huang S X, Höke A, Milbrandt J, DiAntonio A. 2019a. Vincristine and bortezomib use distinct upstream mechanisms to activate a common SARM1-dependent axon degeneration program. JCI Insight 4.
Geisler S, Doan R A, Strickland A, Huang X, Milbrandt J, DiAntonio A. 2016. Prevention of vincristine-induced peripheral neuropathy by genetic deletion of SARM1 in mice. Brain J Neurol 139:3092-3108.
Geisler S, Huang S X, Strickland A, Doan R A, Summers D W, Mao X, Park J, DiAntonio A, Milbrandt J. 2019b. Gene therapy targeting SARM1 blocks pathological axon degeneration in mice. J Exp Med 216:294-303.
Gerdts J, Summers D W, Sasaki Y, DiAntonio A, Milbrandt J. 2013. Sarm1-Mediated Axon Degeneration Requires Both SAM and TIR Interactions. J Neurosci 33:13569-13580.
Gilley J, Coleman M P. 2010. Endogenous Nmnat2 is an essential survival factor for maintenance of healthy axons. PLOS Biol 8:e1000300.
Gilley J, Jackson O, Pipis M, Estiar M A, Al-Chalabi A, Danzi M C, van Eijk K R, Goutman S A, Harms M B, Houlden H, lacoangeli A, et al. 2021. Enrichment of SARM1 alleles encoding variants with constitutively hyperactive NADase in patients with ALS and other motor nerve disorders. eLife 10:e70905.
Gomez-Sanchez J A, Carty L, Iruarrizaga-Lejarreta M, Palomo-Irigoyen M, Varela-Rey M, Griffith M, Hantke J, Macias-Camara N, Azkargorta M, Aurrekoetxea I, et al. 2015. Schwann cell autophagy, myelinophagy, initiates myelin clearance from injured nerves. J Cell Biol 210:153-168.
Gould S A, Gilley J, Ling K, Jafar-Nejad P, Rigo F, Coleman M. 2021a. Sarm 1 haploinsufficiency or low expression levels after antisense oligonucleotides delay programmed axon degeneration. Cell Rep 37:110108.
Gould S A, White M, Wilbrey A L, Por E, Coleman M P, Adalbert R. 2021b. Protection against oxaliplatin-induced mechanical and thermal hypersensitivity in Sarm1−/− mice. Exp Neurol 338:113607.
Henninger N, Bouley J, Sikoglu E M, An J, Moore C M, King J A, Bowser R, Freeman M R, Brown R H Jr. 2016. Attenuated traumatic axonal injury and improved functional outcome after traumatic brain injury in mice lacking Sarm1. Brain 139:1094-1105.
Huppke P, Wegener E, Gilley J, Angeletti C, Kurth I, Drenth J P H, Stadelmann C, Barrantes-Freer A, Brück W, Thiele H, Nürnberg P, Gärtner J, Orsomando G, Coleman M P. 2019. Homozygous NMNAT2 mutation in sisters with polyneuropathy and erythromelalgia. Exp Neurol 320:112958.
Jessen K R, Mirsky R. 2005. The origin and development of glial cells in peripheral nerves. Nat Rev Neurosci 6:671-682.
Kettleborough R N W, Busch-Nentwich E M, Harvey S A, Dooley C M, de Bruijn E, van Eeden F, Sealy I, White R J, Herd C, Nijman I J, Fényes F, Mehroke S, Scahill C, Gibbons R, Wali N, Carruthers S, Hall A, Yen J, Cuppen E, Stemple D L. 2013. A systematic genome-wide analysis of zebrafish protein-coding gene function. Nature 496:494-497.
Kim Y, Zhou P, Qian L, Chuang J-Z, Lee J, Li C, ladecola C, Nathan C, Ding A. 2007. MyD88-5 links mitochondria, microtubules, and JNK3 in neurons and regulates neuronal survival. J Exp Med 204:2063-2074.
Ko K W, Milbrandt J, DiAntonio A. 2020. SARM1 acts downstream of neuroinflammatory and necroptotic signaling to induce axon degeneration. J Cell Biol 219:e201912047.
Krauss R, Bosanac T, Devraj R, Engber T, Hughes R O. 2020. Axons Matter: The Promise of Treating Neurodegenerative Disorders by Targeting SARM1-Mediated Axonal Degeneration. Trends Pharmacol Sci 41:281-293.
Kwan K M, Fujimoto E, Grabher C, Mangum B D, Hardy M E, Campbell D S, Parant J M, Yost H J, Kanki J P, Chien C-B. 2007. The Tol2kit: a multisite gateway-based construction kit for Tol2 transposon transgenesis constructs. Dev Dyn Off Publ Am Assoc Anat 236:3088-3099.
LeWitt P A. 1980. The neurotoxicity of the rat poison vacor. A clinical study of 12 cases. N Engl J Med 302:73-77.
Lin C-W, Liu H-Y, Chen C-Y, Hsueh Y-P. 2014. Neuronally-expressed Sarm1 regulates expression of inflammatory and antiviral cytokines in brains. Innate Immun 20:161-172.
Liu H, Zhang J, Xu X, Lu S, Yang D, Xie C, Jia M, Zhang W, Jin L, Wang X, Shen X, Li F, Wang W, Bao X, Li S, Zhu M, Wang W, Wang Y, Huang Z, Teng H. 2021. SARM1 promotes neuroinflammation and inhibits neural regeneration after spinal cord injury through NF-κB signaling. Theranostics 11:4187-4206.
Loreto A, Angeletti C, Gu W, Osborne A, Nieuwenhuis B, Gilley J, Arthur-Farraj P, Merlini E, Amici A, Luo Z, Hartley-Tassell L, Ve T, Desrochers L M, Wang Q, Kobe B, Orsomando G, Coleman M P. 2021. Neurotoxin-mediated—potent activation of the axon degeneration regulator SARM1. eLife 10:e72823.
Lukacs M, Gilley J, Zhu Y, Orsomando G, Angeletti C, Liu J, Yang X, Park J, Hopkin R J, Coleman M P, Zhai R G, Stottmann R W. 2019. Severe biallelic loss-of-function mutations in nicotinamide mononucleotide adenylyltransferase 2 (NMNAT2) in two fetuses with fetal akinesia deformation sequence. Exp Neurol 320:112961.
Marion C M, McDaniel D P, Armstrong R C. 2019. Sarm1 deletion reduces axon damage, demyelination, and white matter atrophy after experimental traumatic brain injury. Exp Neurol 321:113040.
Merlini E, Coleman M P, Loreto A. 2022. Mitochondrial dysfunction as a trigger of programmed axon death. Trends Neurosci 45:53-63.
Mo W, Nicolson T. 2011. Both Pre- and Postsynaptic Activity of Nsf Prevents Degeneration of Hair-Cell Synapses. PLOS ONE 6:e27146.
Oehlers S H, Cronan M R, Scott N R, Thomas M I, Okuda K S, Walton E M, Beerman R W, Crosier P S, Tobin D M. 2015. Interception of host angiogenic signalling limits mycobacterial growth. Nature 517:612-615.
Osterloh J M, Yang J, Rooney™, Fox A N, Adalbert R, Powell E H, Sheehan A E, Avery M A, Hackett R, Logan M A, MacDonald J M, Ziegenfuss J S, Milde S, Hou Y-J, Nathan C, Ding A, Brown R H, Conforti L, Coleman M, Tessier-Lavigne M, Züchner S, Freeman M R. 2012. dSarm/Sarm1 Is Required for Activation of an Injury-Induced Axon Death Pathway. Science 337:481-484.
Preibisch S, Saalfeld S, Tomancak P. 2009. Globally optimal stitching of tiled 3D microscopic image acquisitions. Bioinforma Oxf Engl 25:1463-1465.
van Rheenen W, Shatunov A, Dekker A M, Mclaughlin R L, Diekstra F P, Pulit S L, van der Spek RAA, Vosa U, de Jong S, Robinson M R, et al. 2016. Genome-wide association analyses identify new risk variants and the genetic architecture of amyotrophic lateral sclerosis. Nat Genet 48:1043-1048.
Sasaki Y, Kakita H, Kubota S, Sene A, Lee T J, Ban N, Dong Z, Lin J B, Boye S L, DiAntonio A, Boye S E, Apte R S, Milbrandt J. 2020. SARM1 depletion rescues NMNAT1-dependent photoreceptor cell death and retinal degeneration. eLife 9:e62027.
Schindelin J, Arganda-Carreras I, Frise E, Kaynig V, Longair M, Pietzsch T, Preibisch S, Rueden C, Saalfeld S, Schmid B, Tinevez J-Y, White D J, Hartenstein V, Eliceiri K, Tomancak P, Cardona A. 2012. Fiji: an open-source platform for biological-image analysis. Nat Methods 9:676-682.
Tian W, Czopka T, López-Schier H. 2020. Systemic loss of Sarm1 protects Schwann cells from chemotoxicity by delaying axon degeneration. Commun Biol 3:49.
Tsunoda I. 2008. Axonal degeneration as a self-destructive defense mechanism against neurotropic virus infection. Future Virol 3:579-593.
Turkiew E, Falconer D, Reed N, Hoke A. 2017. Deletion of Sarm1 gene is neuroprotective in two models of peripheral neuropathy. J Peripher Nerv Syst JPNS 22:162-171.
Uccellini M B, Bardina S V, Sánchez-Aparicio M T, White K M, Hou Y-J, Lim J K, Garcia-Sastre A. 2020. Passenger Mutations Confound Phenotypes of SARM1-Deficient Mice. Cell Rep 31:107498.
Wu T, Zhu J, Strickland A, Ko K W, Sasaki Y, Dingwall C B, Yamada Y, Figley M D, Mao X, Neiner A, Bloom A J, DiAntonio A, Milbrandt J. 2021. Neurotoxins subvert the allosteric activation mechanism of SARM1 to induce neuronal loss. Cell Rep 37:109872.
Ye J, Coulouris G, Zaretskaya I, Cutcutache I, Rozen S, Madden T L. 2012. Primer-BLAST: a tool to design target-specific primers for polymerase chain reaction. BMC Bioinformatics 13:134.
Zhu C, Li B, Frontzek K, Liu Y, Aguzzi A. 2019. SARM1 deficiency up-regulates XAF1, promotes neuronal apoptosis, and accelerates prion disease. J Exp Med 216:743-756.
Angeletti, C., Amici, A., Gilley, J., Loreto, A., Trapanotto, A. G., Antoniou, C., Merlini, E., Coleman, M. P., and Orsomando, G. (2022). SARM1 is a multi-functional NAD(P)ase with prominent base exchange activity, all regulated bymultiple physiologically relevant NAD metabolites. IScience 25, 103812. https://doi.org/10.1016/j.isci.2022.103812.
Di Stefano, M., Nascimento-Ferreira, I., Orsomando, G., Mori, V., Gilley, J., Brown, R., Janeckova, L., Vargas, M. E., Worrell, L. A., Loreto, A., et al. (2015). A rise in NAD precursor nicotinamide mononucleotide (NMN) after injury promotes axon degeneration. Cell Death and Differentiation 22, 731-742. https://doi.org/10.1038/cdd.2014.164.
Di Stefano, M., Loreto, A., Orsomando, G., Mori, V., Zamporlini, F., Hulse, R. P., Webster, J., Donaldson, L. F., Gering, M., Raffaelli, N., et al. (2017). NMN Deamidase Delays Wallerian Degeneration and Rescues Axonal Defects Caused by NMNAT2 Deficiency In Vivo. Current Biology 27, 784-794. https://doi.org/10.1016/j.cub.2017.01.070.
Essuman, K., Summers, D. W., Sasaki, Y., Mao, X., DiAntonio, A., and Milbrandt, J. (2017). The SARM1 Toll/Interleukin-1 Receptor Domain Possesses Intrinsic NAD+Cleavage Activity that Promotes Pathological Axonal Degeneration. Neuron 93, 1334-1343.e5. https://doi.org/10.1016/j.neuron.2017.02.022.
Figley, M. D., and DiAntonio, A. (2020). The SARM1 axon degeneration pathway: control of the NAD+ metabolome regulates axon survival in health and disease. Current Opinion in Neurobiology 63, 59-66. https://doi.org/10.1016/j.conb.2020.02.012.
Figley, M. D., Gu, W., Nanson, J. D., Shi, Y., Sasaki, Y., Cunnea, K., Malde, A. K. Jia, X., Luo, Z., Saikot, F. K., et al. (2021). SARM1 is a metabolic sensor activated by an increased NMN/NAD⁺ ratio to trigger axon degeneration. Neuron https://doi.org/10.1016/j.neuron.2021.02.009.
Gallanosa, A. G., Spyker, D. A., and Curnow, R. T. (1981). Diabetes Mellitus Associated with Autonomic and Peripheral Neuropathy after Vacor Rodenticide Poisoning: A Review. Clinical Toxicology 18, 441-449. https://doi.org/10.3109/15563658108990268.
Gilley, J., Orsomando, G., Nascimento-Ferreira, I., and Coleman, M. P. (2015). Absence of SARM1 Rescues Development and Survival of NMNAT2-Deficient Axons. Cell Reports 10, 1974-1981. https://doi.org/10.1016/j.celrep.2015.02.060.
Gilley, J., Ribchester, R. R., and Coleman, M. P. (2017). Sarm1 Deletion, but Not WldS, Confers Lifelong Rescue in a Mouse Model of Severe Axonopathy. Cell Rep 21, 10-16. https://doi.org/10.1016/j.celrep.2017.09.027.
Watson, D. F., and Griffin, J. W. (1987). Vacor Neuropathy: Ultrastructural and Axonal Transport Studies. J Neuropathol Exp Neurol 46, 96-108. https://doi.org/10.1097/00005072-198701000-00009.
Zhao, Z. Y., Xie, X. J., Li, W. H., Liu, J., Chen, Z., Zhang, B., Li, T., Li, S. L., Lu, J. G., Zhang, L., et al. (2019). A Cell-Permeant Mimetic of NMN Activates SARM1 to Produce Cyclic ADP-Ribose and Induce Non-apoptotic Cell Death. IScience 15, 452-466. https://doi.org/10.1016/j.isci.2019.05.001.
Arthur-Farraj, Peter, and Michael P. Coleman. 2021. ‘Lessons from Injury: How Nerve Injury Studies Reveal Basic Biological Mechanisms and Therapeutic Opportunities for Peripheral Nerve Diseases’. Neurotherapeutics: The Journal of the American Society for Experimental NeuroTherapeutics. doi: 10.1007/s13311-021-01125-3.
Arthur-Farraj, Peter J., Morwena Latouche, Daniel K. Wilton, Susanne Quintes, Elodie Chabrol, Ambily Banerjee, Ashwin Woodhoo, Billy Jenkins, Mary Rahman, Mark Turmaine, Grzegorz K. Wicher, Richard Mitter, Linda Greensmith, Axel Behrens, Gennadij Raivich, Rhona Mirsky, and Kristján R. Jessen. 2012. ‘C-Jun Reprograms Schwann Cells of Injured Nerves to Generate a Repair Cell Essential for Regeneration’. Neuron 75(4): 633-47. doi: 10.1016/j.neuron.2012.06.021.
Bach, Karen, and Richard Simman. 2022. ‘The Multispecialty Toxin: A Literature Review of Botulinum Toxin’. Plastic and Reconstructive Surgery-Global Open 10(4):e4228. doi: 10.1097/GOX.0000000000004228.
Burness, Celeste B., and Paul L. McCormack. 2016. ‘Capsaicin 8% Patch: A Review in Peripheral Neuropathic Pain’. Drugs 76(1): 123-34. doi: 10.1007/s40265-015-0520-9.
Duchen, L. W., S. Gomez, and L. S. Queiroz. 1981. ‘The Neuromuscular Junction of the Mouse after Black Widow Spider Venom.’ The Journal of Physiology 316(1): 279-91. doi: 10.1113/jphysiol. 1981.sp013787.
Harris, J. B., B. D. Grubb, C. A. Maltin, and R. Dixon. 2000. ‘The Neurotoxicity of the Venom Phospholipases A (2), Notexin and Taipoxin’. Experimental Neurology 161(2): 517-26. doi: 10.1006/exnr.1999.7275.
Lacy, Brian E., Kirsten Weiser, and Abigail Kennedy. 2008. ‘Botulinum Toxin and Gastrointestinal Tract Disorders’. Gastroenterology & Hepatology 4(4): 283-95.
Lim, Erle C. H., and Raymond C. S. Seet. 2010. ‘Use of Botulinum Toxin in the Neurology Clinic’. Nature Reviews Neurology 6(11): 624-36. doi: 10.1038/nrneurol.2010.149.
Rigoni, Michela, and Cesare Montecucco. 2017. ‘Animal Models for Studying Motor Axon Terminal Paralysis and Recovery’. Journal of Neurochemistry 142(S2): 122-29. doi: 10.1111/jnc. 13956.
Simone, D. A., M. Nolano, T. Johnson, G. Wendelschafer-Crabb, and W. R. Kennedy. 1998. ‘Intradermal Injection of Capsaicin in Humans Produces Degeneration and Subsequent Reinnervation of Epidermal Nerve Fibers: Correlation with Sensory Function’. The Journal of Neuroscience: The Official Journal of the Society for Neuroscience 18(21): 8947-59.
Thesleff, S., J. Molgo, and S. Tagerud. 1990. ‘Trophic Interrelations at the Neuromuscular Junction as Revealed by the Use of Botulinal Neurotoxins’. Journal De Physiologie 84 (2): 167-73.
Velasco, Elena, Teresa Gledhill, Cristhian Linares, and Antonio Roschman-González. 2008. ‘[Ultrastructural analysis of mouse levator auris longus muscle intoxicated in vivo by botulinum neurotoxin type A]’. Investigacion Clinica 49(4): 469-86.
Witzemann, V., H. R. Brenner, and B. Sakmann. 1991. ‘Neural Factors Regulate AChR Subunit MRNAs at Rat Neuromuscular Synapses’. The Journal of Cell Biology 114(1): 125-41. doi: 10.1083/jcb.114.1.125.
Moga M A, Dimienescu O G, Balan A, Scarneciu I, Barabas B, Ples L. ‘Therapeutic Approaches of Botulinum Toxin in Gynecology’ Toxins (Basel). 2018 Apr. 21; 10(4): 169. doi: 10.3390/toxins10040169.
Apostolidis A., Dasgupta, P., Denys P., Elneil S., Fowler, C. J., Giannantoni, A., Karsenty, G., Schulte-Baukloh, H., Schurch, B., Wyndaele, J-J., European Consensus Panel, 2009. ‘Recommendations on the use of botulinum toxin in the treatment of lower urinary tract disorders and pelvic floor dysfunctions: a European consensus report’. Eur Urol. 2009 January; 55 (1): 100-19. doi: 10.1016/j.eururo.2008.09.009. Epub 2008 Sep. 17.
Hughes R. O., Bosanac T., Mao X., Engber T. M., DiAntonio A., Milbrandt J., Devraj R., Krauss R., ‘Small Molecule SARM1 Inhibitors Recapitulate the SARM1−/− Phenotype and Allow Recovery of a Metastable Pool of Axons Fated to Degenerate’, Cell Rep. 2021 Jan. 5; 34 (1): 108588, doi: 10.1016/j.celrep.2020.108588.
Bosanac T., Hughes, R. O., Engber, T., Devraj R., Brearley A., Danker, K., Young, K., Kopatz J., Hermann, M., Berthemy A., Boyce S., Bentley J., Krauss R, ‘Pharmacological SARM1 inhibition protects axon structure and function in paclitaxel-induced peripheral neuropathy’; Brain. 2021 Nov. 29; 144(10): 3226-3238. doi: 10.1093/brain/awab184.
Shi Y., Kerry P. S, Nanson J. D., Bosanac T., Sasaki, Y., Krauss R., Saikot F. K., Adams S. E., Mosaiab T., Masic V., Mao X., Rose F., Vasquez E., Furrer M., Cunnea K., Brearley A., Gu W., Luo Z., Brillault L., Landsberg M. J., DiAntonio A., Kobe B., Milbrandt J., Hughes R. O, Ve T.; ‘Structural basis of SARM1 activation, substrate recognition, and inhibition by small molecules’, Mol Cell. 2022 May 5; 82(9): 1643-1659.e10. doi: 10.1016/j.molcel.2022.03.007.
Feldman H. C., Merlini E., Guijas C., DeMeester K. E., Njomen E., Kozina E. M., Yokoyama M., Vinogradova E., Reardon H. T., Melillo B., Schreiber S. L., Loreto A., Blankman J. L, Cravatt, B. F, ‘Selective inhibitors of SARM1 targeting an allosteric cysteine in the autoregulatory ARM domain’, Proc Natl Acad Sci USA 2022 Aug. 30; 119(35):e2208457119. doi: 10.1073/pnas.2208457119.
Babetto, Elisabetta, Keit Men Wong, and Bogdan Beirowski. “A glycolytic shift in Schwann cells supports injured axons.” Nature neuroscience 23. 10 (2020): 1215-1228.
Mutschler, Clara, et al. “Schwann cells are axo-protective after injury irrespective of myelination status in mouse Schwann cell-neuron cocultures.” Journal of Cell Science 136.18 (2023).
Szretter, Kristy J., et al. “The immune adaptor molecule SARM modulates tumor necrosis factor alpha production and microglia activation in the brainstem and restricts West Nile Virus pathogenesis.” Journal of virology 83.18 (2009): 9329-9338.
Barrette, Benoit, et al. “Requirement of myeloid cells for axon regeneration.” Journal of Neuroscience 28.38 (2008): 9363-9376.
Buonvicino, Daniela, et al. “Identification of the nicotinamide salvage pathway as a new toxification route for antimetabolites.” Cell chemical biology 25.4 (2018): 471-482.
Xie, Na, et al. “NAD+ metabolism: pathophysiologic mechanisms and therapeutic potential.” Signal transduction and targeted therapy 5.1 (2020): 227.

1. A SARM1 agent for use as a medicament.

2. A SARM1 agent for use in a method of treating and/or preventing neurological, ophthalmological, dermatological, gastroenterological, colorectal, urological, gynaecological, rheumatological, orthopaedic, dental, and/or ear nose and throat disease.

3. The SARM1 agent for use according to claim 2, wherein the disease is defined by autonomic dysfunction, neuropathic pain, dystonia, spasticity, dyskinesia, sphincter dysfunction, genitourinary dysfunction and/or a skin condition.

4. The SARM1 agent for use according to claim 2 or 3, wherein the:
(i) autonomic dysfunction comprises:
excessive sweating (hyperhidrosis) and/or excessive saliva production (sialorrhoea),
autonomic dysfunction caused by:
inherited and/or genetic neurological diseases comprising;
hereditary autonomic neuropathy, sensory autonomic neuropathy; and/or
degenerative neurological diseases comprising;
amyotrophic lateral sclerosis, Parkinson's disease, parkinsonism, multiple system atrophy, cerebral palsy and/or degenerative neurological diseases due to injury; and/or
disease of the autonomic nervous system from an acquired disorder (secondary dysautonomia) comprising;
infections (COVID and Long COVID), autoimmune conditions, postural orthostatic tacycardia syndrome (POTS), endocrine conditions comprising
diabetes and/or thyroid dysfunction; and/or
small fibre neuropathy; and/or
connective tissue diseases; and/or
amyloidosis; and/or
peripheral nerve injuries; and/or
surgical repair of peripheral nerve injuries and/or
medications; and/or
idiopathic autonomic dysfunction; and/or
(ii) neuropathic pain comprises:
peripheral neuropathic pain; chronic back pain; cancer related pain; myofascial pain; fibromyalgia; complex regional pain disorder; chronic pelvic pain; dyspareunia; vulvodynia; and/or painful bladder syndrome (interstitial cystitis); and/or
neuropathic pain caused by:
diabetic neuropathy neuralgia post peripheral nerve injuries; infective and post-infective neuralgias; metabolic neuropathies; small fibre neuropathy; endocrine conditions; genetic neuropathies; paraproteinaemic neuropathies; paraneoplastic neuropathies; neoplastic neuropathies; vasculitis; auto-immune, inflammatory and demyelinating neuropathies; idiopathic neuropathies, peripheral nerve injuries and/or surgical repair of peripheral nerve injuries; and/or
(iii) dystonia comprises:
primary dystonia;
dystonia plus conditions;
dystonia occurring with neurodegenerative diseases; and/or
secondary dystonia occurring due to:
central nervous system (CNS) trauma, congenital malformation, genetic and chromosomal disorders, infection, neoplasm, ischaemic; haemorrhagic stroke, inflammation, demyelination, drugs, toxins, and/or metabolic disturbance; and/or
(iv) spasticity comprises
upper limb spasticity, lower limb spasticity; bladder spasticity/neurogenic bladder; spastic disorders of the oesophagus; and/or
spasticity caused by:
demyelinating conditions; congenital malformation; chromosomal disorders; genetic conditions; CNS infections; neoplasm; drugs; toxins; metabolic disturbance; paraneoplastic disorders; post-infections conditions; autoimmune conditions; endocrine conditions, peripheral nerve injuries and/or surgical repair of peripheral nerve injuries; and/or
(v) dyskinesia comprises:
hemifacial spasm and facial nerve synkinesis; palatal myoclonus/tremor; and/or
dyskinesia caused by:
tic disorders; tremor; myokymia, trauma, infection, autoimmune conditions; neuromyotonia caused by autoimmune and/or hereditary conditions; and/or
(vi) sphincter dysfunction is caused by:
dysfunction of bodily sphincters; pyloric sphincter; sphincter of Oddi dysfunction leading to gastrointestinal dysmotility; pancreatic sphincter; urethral sphincter; anal fissure; and/or Hirshsprungs disease;
(vii) genitourinary dysfunction comprises:
vaginismus; and/or voiding dysfunction; and/or
(viii) skin condition comprises:
an ulcer; acne; and/or raynauds phenomenon/disease;
(ix) orthopaedic disease comprises:
sports injuries; posttraumatic elbow stiffness; club foot; and/or piriformis syndrome; and/or
(x) dental disease comprises:
odontalgia; and/or root canal treatment.

5. A non-therapeutic method of:
(i) removing and/or preventing skin changes;
(ii) contouring; and/or
(iii) improving oily skin;
the method comprising administering a SARM1 agent.

6. The non-therapeutic method of claim 5, wherein skin changes comprise one or more of skin wrinkles, frown/glabellar lines, forehead lines, lateral canthal lines/crow's feet, horizontal nasal bridge lines, neck bands, platysmal neck lines, smoker's lines, bunny lines, top lip lines, ptosis of the lateral commissure, marionette lines, gummy smile, chin dimples, peau d'orange chin, pitted skin, eye brow sagging, eyelid sagging, lip sagging, square jaw, and a downturned nasal tip.

7. The non-therapeutic method of claim 5 or 6, wherein contouring comprises (i) shaping lip, cheek, jaw, and/or temple; and/or (ii) eyebrow contour.

8. The SARM1 agent for use according to any one of claims 1-4 or the method of claims 6-7, wherein the SARM1 agent:
the SARM1 agent activates SARM1 and causes neurodegeneration and/or neuronal dysfunction; and/or
causes SARM1 dependent neurodegeneration and/or neuronal dysfunction.

9. The SARM1 agent for use according to any one of claims 1-5 or the method of claims 6-8, wherein the SARM1 agent activates SARM1 by:
binding to SARM1;
increasing intracellular levels of NMN;
decreasing intracellular NAD levels; and/or
increasing an intracellular NMN/NAD ratio.

10. The SARM1 agent for use according to any one of claims 8-9 or the method of any one of claims 8-9, wherein the neurodegeneration comprises axonal, terminal aborization, or synaptic terminal degeneration and/or neurolytic death, optionally wherein the axonal degeneration, terminal aborization and/or synaptic terminal and/or neurolytic death is reversible and/or temporary, further optionally wherein after the neurodegeneration neuronal regeneration occurs.

11. The SARM1 agent for use according to any one of claims 8 to 10, or the method of any one of claims 8 to 10, wherein the neurodegeneration and/or neuronal dysfunction is reversible by a reversal agent.

12. The SARM1 agent for use according to any one of claims 8 to 11 or the method of any one of claims 8 to 11, wherein neurodegeneration and/or neuronal dysfunction:
(i) occurs in cells expressing SARM1, optionally wherein the cells expressing SARM1 comprise neurons; and
(ii) does not occur in cells lacking SARM1, or expressing functionally insignificant amounts of SARM1 protein, optionally wherein the cells lacking SARM1 or expressing low and/or functionally insignificant amounts of SARM1, comprise Schwann cells; muscle cells, skin cells (such as dermal fibroblasts), HEK293 cells, HEK293T cells, Hela cells, SK mel 2 cells, SK mel 5 cells, SK mel 28 cells, CAPAN cells, JURKAT cells, C6 cells, HL-60 cells, LP-1 cells, U937 cells, MEWO cells, monocytes, macrophages, and/or RAW264.7 cells.

13. The SARM1 agent for use according to any one of claims 1-4 and 6-12 or the method of any one of claims 5 to 12, wherein the SARM1 agent is selected from vacor; vacor mononucleotide (VMN); 3-acetylpyridine (3-AP); 3-acetylpyridine mononucleotide (3-APMN); 2-aminopyridine (2-AnP); 2-AnP mononucleotide (2-AnPMN); nicotinamide mononucleotide (NMN), sulfo-ara-F-NMN (CZ-48), sulfo-ara-F-VMN; sulfo-ara-F-3-APMN; sulfo-ara-F-2-AnPMN, S-NMN, ara-F-NMN (CZ-17); pyridines and molecules containing pyridine rings; vacor riboside (VR), vacor riboside (VR) analogs, nicotinamide (NAM) analogs; nicotinamide riboside (NR), nicotinamide riboside (NR) analogs; nicotinic acid (NA) analogs; nicotinic acid riboside (NaR) analogs, nicotinic acid mononucleotide (NaMN) analogs, molecules that increase intracellular levels of NMN, decrease NAD levels and/or increase NMN/NAD ratio resulting in SARM1 activation; NMNAT1-2-3 inhibitors or molecules that reduce NMNAT1-2-3 levels; and/or metabolites, analogs and derivatives thereof.

13A. The SARM1 agent for use according to claim 13 or the method of claim 13 wherein the analogs and derivatives increase permeability and/or solubility of the SARM1 agent.

14. The SARM1 agent for use according to any one of claims 1-4 and 6-13A or the method of any one of claims 6-13A, wherein the SARM1 agent is administered locally, optionally by:
(i) a transdermal patch or
(ii) injection into tissue such as (a) the skin or muscle, optionally intravesical injection into detrusor muscle/urethral sphincter, with or without ultrasound, radiographic and/or electromyographic guidance, (b) a neuroma, optionally for peripheral nerve regeneration and surgical nerve repair, and/or (c) a root canal, optionally for use in dental surgery;
(iii) endoscopic injection such as into the oesophogeal sphincter, pyloric sphincter, sphincter of Oddi; cystoscopic injection into the urethral sphincter;
(iv) injection into salivary glands such as sublingual and submandibular glands; or
(v) topical administration.

15. The SARM1 agent for use according to one of claims 1-4 and 6-14 or the method of any one of claims 5 to 14, wherein two or more SARM1 agents as according to any one of claims 8 to 14 are co-administered, optionally wherein an additional agent such as a reversal agent, an analgesic and/or an anaesthetic is administered.

16. A composition comprising a SARM1 agent according to any one of claims 8 to 13; optionally wherein the composition is a pharmaceutical composition and further comprises at least one pharmaceutically acceptable excipient, diluent, and/or carrier.

17. The composition according to claim 16, for use according to any one of claims 1 to 4, and 8 to 14.

18. The composition according to claim 16 or for use according to claim 17, wherein the composition is a topical composition; optionally wherein the composition is a cream or ointment.

19. The method according to anyone of claims 5 to 15, wherein the SARM1 agent is comprised in a composition according to claim 16 or 18.

20. A syringe comprising a SARM1 agent according to any one of claims 8 to 13 or a composition according to claim 16 or 18.

21. A transdermal patch comprising a SARM1 agent according to any one of claims 8 to 13 or a composition according to claim 16 or 18.

22. A kit of parts comprising:
a SARM1 agent according to any one of claims 8 to 13 or a composition according to claim 16 or 18; and
an applicator configured for administration of the SARM1 agent.

23. The kit of parts according to claim 22, wherein the applicator comprises a transdermal patch according to claim 21 or a syringe according to claim 20.
